(19) United States
(12) Patent Application Publication
(10) Pub. No.: US 2010/0292439 A1
(43) Pub. Date: Nov. 18, 2010

(54) Use of Functionalized Onium Salts for Peptide Synthesis

(75) Inventors: Michel Vaultier, Chateaugiron (FR); Céline Roche, Brunoy (FR); Saïd Gmouh, Rennes (FR); Alain Commercon, Vitry Sur Seine (FR)

Correspondence Address: YOUNG & THOMPSON, 209 Madison Street, Suite 500, Alexandria, VA, 22314, US

(73) Assignees: CENTRE NATIONAL DE LA RECHERCHE SCIENTIFIQUE`, Paris Cedex (FR); UNIVERSITE DE RENNES, Rennes (FR)

(21) Appl. No.: 11/884,478

(22) PCT Filed: Jul. 5, 2006
(86) PCT No.: PCT/FR2006/001597

§ 371 Date: Mar. 5, 2010

(51) Int. Cl. C07K 1/02; C07D 211/62; C07C 271/22

(52) U.S. Cl. 530/341; 530/333; 530/338; 546/245; 560/29

(57) ABSTRACT

A subject of the invention is the use of a salt with a dedicated task of formula (I):

A⁺-L-R—OY, X⁻

as soluble support for peptide synthesis, in which:
X⁻ represents a functional or non-functional anion,
Y represents either a hydrogen atom, or a —COOR₁ group, R₁ representing in particular an alkyl group comprising 1 to 20 carbon atoms,
A⁺ represents a cationic entity,
L represents an arm, in particular an alkyl group of 3 to 20 carbon atoms,
R represents in particular a group of formula —C(Rₐ)(R_{b})—, Rₐ and R_{b} representing independently of one another in particular a hydrogen or an alkyl group, comprising 1 to 20 carbon atoms.

A subject of the present invention is the use of functionalized onium salts for peptide synthesis, in particular by reverse-route, direct-route synthesis or by convergent synthesis.

Two peptide synthesis techniques can be implemented: unsupported synthesis in solution and supported synthesis.

The first method involves assembling the peptides by coupling the different amino acids in solution. This approach is laborious as each stage requires complex and expensive purification. Supported peptide synthesis was therefore developed in order to overcome these problems.

In 1963, Merrifield introduced solid-supported peptide synthesis [SSPS(R. B. Merrifield, J. Am. Chem. Soc., 1963, 85, 2149)]. Four stages are associated with this method: grafting of a substrate onto a resin; modification of the grafted structure; cleavage of the synthesized molecule from its support and analysis and optional purification of the molecule. Numerous advantages are associated with this technique [S. R. Wilson, A. W. Czarnik, “Combinatorial Chemistry: Synthesis and Application”, John Wiley and Sons New York, 1997; I. M. Charken, K. D. Janda, “Molecular Diversity and Combinatorial Chemistry”, American Chemical Society, Washington, D.C., 1996; R. E. Sammelson, M. J. Kurth, Chem. Rev. 2001, 101, 137]: purification, carried out by simple washing processes, is very easy, which makes automation possible; an excess of reagents can be used in order to make the reactions quantitative (typically 4 to 5 equivalents) and the parallel synthesis or “split and mix” techniques can be adapted. This methodology also has drawbacks: the prices of the functionalized resins are very high and their specific load is very low (often less than 1 mmol/g of resin, rarely reaching 2 mmol/g). Moreover, the reactions take place under heterogeneous conditions and the methods for monitoring the reaction are few and often associated with a prior cleavage from the resin (a method which can be destructive). Moreover, independently of the methods indicated above, the peptides can be prepared by a linear strategy (direct route or by reverse route) or by a convergent strategy. More precisely, convergent peptide synthesis is based on the condensation of fragments and convergent solid phase peptide synthesis (CSPPS) has been developed [P. Lloyd-Williams, F. Albericio, E. Giralt, Tetrahedron. 1993, 49, 48, 11065; K. Barlos, D. Gatos, “Fmoc Solid Phase Peptide Synthesis, A Practical Approach”, Oxford University Press, 2000, chapter 9, “Convergent Peptide Synthesis”, 215] or synthesis by solid phase fragment condensation (SPFC) [H. Benz, Synthesis, 1993, 337; B. Riniker, A. Flörsheimer, H. Fretz, P. Sieber, B. Kamber, Tetrahedron, 1993, 49, 41, 9307].

According to Merrifield's technique, the convergent synthesis (by fragments or blocks) of peptides is not possible without prior cleavage, and it is impossible to separate the expected supported molecules from the by-products grafted onto the resin (originating from incomplete reactions or secondary reactions). After cleavage, a mixture of products is obtained the final purification of which by reversed-phase HPLC (high pressure liquid chromatography) of the peptide does not always make it possible to separate the peptides having truncated chains or comprising deletions, as well as the diastereoisomers formed by epimerization during the synthesis.

To date, no industrializable convergent synthesis process exists involving the formation of peptide bonds, either on solid support, or on soluble support.

Another range of supports was therefore developed. The use of soluble polymers [D. J. Gravert, K. D. Janda, Chem. Rev. 1997, 97, 489; P. Wentworth, K. D. Janda, Chem. Comm., 1999, 1917; P. M. Fischer, D. I. Zheleva, J. Peptide Sci., 2002, 8, 529] makes it possible to carry out the reactions under homogeneous conditions while retaining the possibility of easy purification by simple precipitation of the polymer by the addition of an appropriate solvent then filtration of the reaction medium and washing in order to eliminate the excess reagents and the by-products. Polyethylene glycols (PEG 2000 and 5000 in particular) are the soluble polymers most used for peptide synthesis on soluble polymer. However, various problems are associated with this methodology. In fact, in order to have the required physico-chemical properties, the polymers must have a mass comprised between 2000 and 20000 daltons, which means a very low specific load (0.05 to 0.5 mmol/g for monobranched polymers); the purification of the products is often laborious (in particular because of co-precipitation problems); automation is more difficult than in solid-support synthesis (very viscous solutions, time-consuming precipitation and recrystallization operations, necessity to carry out several successive couplings in order to produce quantitative reactions); a poor solubilization of the PEG is observed for large peptides (aggregation of the peptide chains); and, as in solid-phase synthesis, it is impossible to separate the expected supported molecules from the by-products grafted to the polymer and it is not always possible to completely purify the peptide cleaved by reversed-phase HPLC. In practice, the use of soluble polymers for peptide synthesis remains rare.

The synthesis of peptides with less than five amino acids is usually carried out in solution whereas solid-support synthesis is used for longer peptides. The latter is appropriate for producing small quantities of peptides, but for the scale-up (industrial scale), in particular when kilograms are necessary for industrial productions, conventional synthesis in solution remains most suitable. So-called “hybrid” techniques also exist which combine solid-support synthesis and synthesis in solution (K. Barlos, D. Gatos, Biopolymers, 1999, 51, 266).

Another alternative developed recently involves using fluorinated supports for peptide synthesis [M. Mizuno, K. Goto, T. Miura, D. Hosaka, T. Inazu, Chem. Comm., 2003, 972; M. Mizuno, K. Goto, T. Miura, T. Matsuura, T. Inazu, Tetrahedron Lett., 2004, 45, 3425]. The peptide is grafted onto a fluorinated support. The reactions take place in standard organic solvent then extraction by a fluorinated solvent makes it possible to selectively extract the peptide carrying the fluorinated group. This technique makes it possible to combine the advantages of solid-support synthesis (easy purification, use of a large number of reagent equivalents in order to produce quantitative reactions) and standard synthesis in solution (purification of the possible intermediates, monitoring of the reactions by NMR, TLC (thin-layer chromatography), MS (mass spectrometry), possibility of carrying out reactions on a large scale). However two major drawbacks are associated with this method. On the one hand it uses fluorinated solvents, the synthesis of which is not environmentally friendly, and, on the other hand, the supported peptide must contain a significant percentage by mass of fluorine (greater than 40% by mass) in order to allow correct purification (otherwise emulsions or precipitations of the peptide are observed during the extraction), which means that this technology is only valid for small peptides.

Current peptide synthesis technologies therefore have limitations. The development of novel peptide synthesis technologies is therefore necessary.

Moreover, in the novel ILSOS (ionic liquid supported organic synthesis) and OSSOS (onium salt supported organic synthesis) technologies, as described in the international applications WO 2004/029004 and WO 2005/005345 respectively, and developed for organic synthesis, the possibility of using these methods within the framework of peptide synthesis is not reported.

Ionic liquids (P. Wasserscheid, T. Welton, “Ionic Liquids in Synthesis”, Wiley-VCH, 2003) are low-temperature liquid salts (melting point<100° C.). A very large number of possible uses have been demonstrated: novel solvents for synthesis and catalyses, catalysts in certain reactions, liquid media with a specific task, etc. They have certain useful physico-chemical properties such as a high thermal stability, very low vapour pressures, a significant solubilizing power both of organic molecules and salts or polymers. They are not very inflammable, they are recyclable and their solvent properties can be adjusted at will by varying the nature of the cations and anions.

The functionalized onium salts (or with a specific task or dedicated task) have properties which allow their use as soluble supports for organic synthesis, parallel synthesis and combinatorial chemistry. In fact, these are perfectly defined entities with a low molecular weight which can be characterized by all the physico-chemical methods. They are soluble in a large range of non-functional ionic liquids then serving as liquid matrix leading to ionic liquids with a dedicated task. They are also soluble in a large number of organic solvents and insoluble in others, this solubility depending essentially on the associated anion. This makes it possible to purify them by simple washing and therefore to use an excess of reagents. Moreover, their high thermal stability makes it possible to eliminate the excess reagents by vacuum distillation. Finally, their synthesis is simple, the cost is low and their synthesis on a large scale is possible.

A purpose of the present invention is to provide novel functionalized onium salts intended to be used within the framework of peptide synthesis.

A purpose of the present invention is also to provide a reverse-route, direct-route or convergent peptide synthesis process, by the use of functionalized ionic liquids.

The different aspects are achieved by using the functionalized onium salts as soluble supports.

More precisely, the present invention relates to the use of an onium salt with a dedicated task of formula (I):

A⁺-L-R—OY, X⁻ (I)

as soluble support for peptide synthesis, in which:
X⁻ represents an anion, functional or non-functional, chosen in particular from Cl⁻, Br⁻, I⁻, BF₄⁻, CF₃SO₃⁻, N(SO₂CF₃)₂⁻, PF₆⁻, CH₃CO₂⁻, CF₃CO₂⁻, RαCO₂, R_{F}CO₂⁻, R_{α}SO₃⁻, R_{F}SO₃⁻, R_{α}SO₄⁻, (R_{α})₃₋ₓPO₄^{x-}, x representing an integer equal to 1, 2 or 3, AlCl₄⁻, SnCl₃⁻, ZnCl₃⁻, R_{α} representing an alkyl group comprising 1 to 20 carbon atoms, R_{F} representing a perfluoroalkyl group comprising 1 to 20 carbon atoms,
Y represents:
   either a hydrogen atom, the salt of formula (I) then comprising a cation functionalized by an alcohol function and corresponding to the following formula (I_{D}): A⁺-L-R—OH, X⁻,
   or a —COOR₁ group, R₁ representing an alkyl group comprising 1 to 20 carbon atoms or an aryl group comprising 6 to 30 carbon atoms, or a perfluoroalkyl group comprising 1 to 20 carbon atoms, said alkyl or aryl groups being optionally functionalized, R₁ representing in particular —CHCl—CCl₃ or

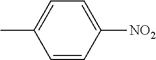

the salt of formula (I) then comprising a cation functionalized by a mixed carbonate function and corresponding to the following formula (I_{I}):

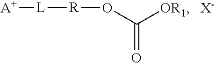

A⁺ represents a cationic entity, in particular chosen from pyridinium, imidazolium, ammonium, phosphonium or sulphonium cations, cyclic or non-cyclic, substituted or non-substituted, and preferably ammonium or phosphonium,
L represents an arm, in particular a linear or branched alkyl group, or aralkyl or alkaryl comprising 3 to 20 carbon atoms,
R represents a group chosen from the following groups:
   a group of formula —C(Rₐ)(R_{b})—, Rₐ and R_{b} representing independently of one another a hydrogen atom or a linear or branched alkyl group, comprising 1 to 20 carbon atoms, the group of formula —C(Rₐ)(R_{b})— preferably representing a —CH₂—, —CH(Me)- or —C(Me)₂- group,
   a group of formula -T-Ar₁—CH(R_{c})—, in which:
      T is chosen from one of the following groups: CH₂, O, S and NR_{d}, R_{d} representing a hydrogen atom or a linear or branched alkyl group, comprising 1 to 20 carbon atoms,
      Arᵢ represents an aromatic group of the following formula:

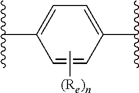

n representing an integer equal to 0, 1, 2, 3 or 4 ,
Rₑ representing either a linear or branched alkyl group comprising 1 to 12 carbon atoms, in particular a methyl group, or an alkoxy group comprising 1 to 12 carbon atoms, in particular a methoxy, ethoxy, propyloxy, isopropyloxy, butyloxy, isobutyloxy or tertiobutyloxy group,
R_{c} represents either a hydrogen atom, or a linear or branched alkyl group, comprising 1 to 20 carbon atoms, or an aromatic group Ar₂ of the following formula:

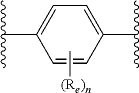

m representing an integer equal to 1, 2, 3, 4, or 5
R_{f} representing either a linear or branched alkyl group, comprising 1 to 12 carbon atoms, in particular a methyl group, or an alkoxy group comprising 1 to 12 carbon atoms, in particular a methoxy, ethoxy, propyloxy, isopropyloxy, butyloxy, isobutyloxy or tertiobutyloxy group.

The Inventors have surprisingly found that the salts with a dedicated task of formula (I) could be used as soluble supports
in direct-route or reverse-route peptide synthesis, producing yield and purity results at least as impressive as those obtained with the techniques of the state of the art and
in convergent peptide synthesis producing very much improved results compared with the techniques of the state of the art, which frees convergent peptide synthesis from the limitations encountered to date.

The expression “salt with a dedicated task” designates the ammonium, phosphonium, sulphonium salts, as well as all the salts resulting from the quaternization of an amine, a phosphine, an arsine, a thioether or a heterocycle containing one or more of these heteroatoms, and carrying at least one organic function Fᵢ or F′ᵢ. This expression also designates an onium salt the cation of which as defined above is not functionalized but the anion of which carries a function F′ᵢ. This expression can also designate a salt the anion and the cation of which carry at least one organic function.

The expression “soluble support” designates a functional onium salt serving as an “anchor” in order to carry out, in solution, successive conversions of a molecule attached by the function. This anchor confers properties on the attached molecule (therefore finally to the group formed by the anchor and the attached molecule) which make it possible to purify easily by washing, evaporation or any other technique. This could not be done easily with molecules which are volatile and/or soluble in the usual solvents for example. By using this technique, it is possible to use excess reagents, for example, as in the case of the insoluble Merrifield resins. A soluble support must by definition be soluble in a solvent or in another ionic liquid. This confers the advantage of carrying out the reactions in solution and being able to monitor progress using analysis techniques used in a standard fashion in the field of peptide synthesis. A soluble support of the onium salt type with a dedicated task must also be recoverable at the end of the conversions. In other words, the molecules synthesized on this support must be able to be easily cleaved. Moreover, the skeleton of the soluble support must not react with the reagents used, the reactions taking place selectively on the functions attached to the basic skeleton.

The present invention relates to the use of an onium salt with a dedicated task as defined above for peptide synthesis comprising in particular from 2 to 30 amino acids, and preferably from 2 to 25, in particular from 10 to 25 amino acids, or from 15 to 20 amino acids.

Advantageously, the abovementioned arm L represents an alkyl, aralkyl or alkaryl group comprising 3 to 20 carbon atoms, and in particular comprising 3, 4, 5, 6, 7, 8, 9, 10, 11, 12, 13, 14, 15, 16, 17, 18, 19 or 20 carbon atoms. If the arm L contains less than 3 carbon atoms, problems of stability of the reagents supported with such an arm are observed due to the proximity of the cation.

The present invention relates to the use as defined above, for peptide synthesis, of azapeptides or pseudopeptides, said peptides, azapeptides or pseudopeptides comprising at least one peptide bond and/or at least one azapeptide bond and/or at least one pseudopeptide bond, and optionally comprising at least one α-hydrazino acid, α-amino acid or Ω-amino acid unit, in particular β-amino acid or γ-amino acid, cyclic or linear.

The α-hydrazino acids can be represented for example by the following formula: R—HN—NH—(CHR′)ₙ—COOH, R and R′ representing an alkyl or aryl or aralkyl or alaryl group comprising 1 to 20 carbon atoms and n varying from 1 to 10.

The present invention relates to the use as defined above, for the grafting of at least one amino acid
of formula HOOC—[CH(R′)]ₚ—NHGP, onto a compound of formula (I_{D}) as defined below,
   p representing an integer varying from 1 to 20,
   R′ representing an amino acid residue, said amino acid being a non-functional amino acid or a functional amino acid (such as lysine, tyrosine, threonine, serine etc.) the function or functions of which are protected and therefore do not serve as an anchorage point for the support,
   GP representing a protective group of the amine function, with the exception of Boc, in particular Fmoc, Cbz, Z, SO₂R_{g}, R_{g} representing a linear or branched alkyl group comprising 1 to 20 carbon atoms, a substituted or non-substituted aryl group, a perfluoroalkyl group comprising 1 to 20 carbon atoms,
   in order to obtain a compound of the following formula:

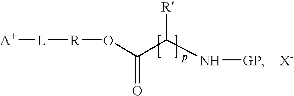

A⁺, L and R being as defined above,
p, R′ and GP being as defined above,
or of formula R₂—NH—[CH(R′)]ₚ—COOR₃, onto a compound of formula (I_{I}) as defined below,
   p representing an integer varying from 1 to 20,
   R′ representing an amino acid residue as defined above, i.e. functional or non-functional,
   R₂ representing a linear or branched alkyl group, comprising 1 to 20 carbon atoms and being able to form a ring with the R′ group, the nitrogen atom carrying the R₂ group and the carbon atom carrying the R′ group, said ring comprising 3 to 20 members, in particular 5 or 6 members, and
   R₃ representing a hydrogen atom or a protective group of the terminal acid function of the amino acid, and being chosen from one of the following groups: a linear or branched alkyl group, comprising 1 to 20 carbon atoms, in particular methyl or tertiobutyl, a benzyl group or an Si(ORₕ)₃ group, Rₕ representing a linear or branched alkyl group of 1 to 20 carbon atoms, and representing in particular a tertiobutyl group,

to obtain a compound of the following formula: X⁻
A⁺, L and R being as defined above,
p, R₂, R′ and R₃ being as defined above.

The use of an amino acid of formula HOOC—[CH(R′)]ₚ—NHGP on a compound of formula (I_{D}) corresponds to direct peptide synthesis and makes it possible to obtain the formation of an ester after grafting onto the support.

The use of an amino acid of formula R₂—NH—[CH(R′)]ₚ—COOR₃ on a compound of formula (I_{I}) corresponds to reverse peptide synthesis and makes it possible to obtain the formation of a carbamate after grafting onto the support.

Given the definition of R₃, the latter can represent H or a protective group of the terminal acid function of the amino acid. Thus, preferably, R₃ represents H in the case where the amino acid is a β-amino acid or a superior homologue (γ, δ, etc. . . . ) in which the nucleophilic nature of the nitrogen atom is sufficient. In the case where the amino acid is an α-amino acid, R₃ preferably represents a protective group, due to the nucleophilic nature of the nitrogen and the insufficient solubility of the non-esterified α-amino acids.

The amino acids being bifunctional compounds, two routes can be envisaged for peptide synthesis: the direct route C→N (the amino acid is grafted onto the support by its acid function and its amine function is involved in the peptide coupling reaction) and the reverse route N→C (the amino acid is grafted onto the support by its amine function via a carbamate function and its acid function is involved in the peptide coupling reaction).

The present invention also relates to the use as defined above, of a salt with a dedicated task of formula

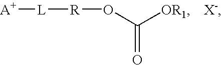

for reverse-route peptide synthesis, in which:
A⁺, X⁻ and L are as defined above,

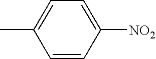

R₁ represents in particular a —CHCl—CCl₃ group or
R represents a group of formula —C(Rₐ)(R_{b})—, Rₐ and R_{b} representing independently of one another a hydrogen atom or a linear or branched alkyl group, comprising 1 to 20 carbon atoms, the group of formula —C(Rₐ)(R_{b})— preferably representing a —CH₂—, —CH(Me)- or —C(Me)₂- group.

According to an advantageous embodiment, the present invention relates to the use as defined above, of a salt with a dedicated task of formula A⁺-L-R—OH, X⁻ for direct route peptide synthesis, in which:
A⁺, X⁻ and L are as defined above,
R represents a group of formula -T-Ar₁—CH(R_{c})—, in which:
T is chosen from one of the following groups: CH₂, O, S and NR_{d}, in particular O, R_{d} representing a hydrogen atom or a linear or branched alkyl group, comprising 1 to 20 carbon atoms,
Ar₁ represents an aromatic group of the following formula:

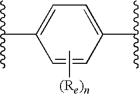

n representing an integer equal to 0, 1 or 2, 3, or 4
Rₑ representing either a linear or branched alkyl group, comprising 1 to 20 carbon atoms, in particular a methyl group, or an alkoxy group comprising 1 to 20 carbon atoms, in particular a methoxy, ethoxy, propyloxy, isopropyloxy, butyloxy, isobutyloxy or tertiobutyloxy group,
R_{c}, represents either a hydrogen atom, or a linear or branched alkyl group, comprising 1 to 20 carbon atoms, or an aromatic group Ar₂ of the following formula:

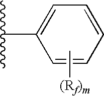

m representing an integer equal to 1, 2, 3 4 or 5
R_{f} representing either a linear or branched alkyl group, comprising 1 to 20 carbon atoms, in particular a methyl group, or an alkoxy group comprising 1 to 20 carbon atoms, in particular a methoxy, ethoxy, propyloxy, isopropyloxy, butyloxy, isobutyloxy or tertiobutyloxy group.

The present invention also relates to the use as defined above, for the peptide synthesis by convergent route, of a salt with a dedicated task A⁺-L-R—OY, X⁻ of formula (I) as defined above, and of a salt with a dedicated task of formula Aᵢ⁺-Lᵢ-Rᵢ—OH, Xᵢ⁻, the elements A⁺, L, R, Y and X⁻ being as defined above, and the elements Aᵢ⁺, Lᵢ, Rᵢ, and Xᵢ⁻ having the definitions given above in connection with A⁺, L, R and X⁻ respectively, A⁺-L-R and Aᵢ⁺-Lᵢ-Rᵢ, being able to be identical or different.

The present invention relates to the use as defined above, characterized in that A⁺ is chosen from the cyclic or non-cyclic quaternary ammonium cations.

According to an advantageous embodiment, the use as defined above is characterized in that L represents a linear alkyl chain comprising 4 or 5 carbon atoms.

The present invention also relates to the use as defined above, characterized in that the anion X⁻ is PF₆⁻ or NTf₂⁻.

The present invention also relates to the use as defined above, for reverse-route peptide synthesis, comprising the use of a salt with a dedicated task of formula (I_{I}) as defined above, the cation corresponding to one of the following formulae:

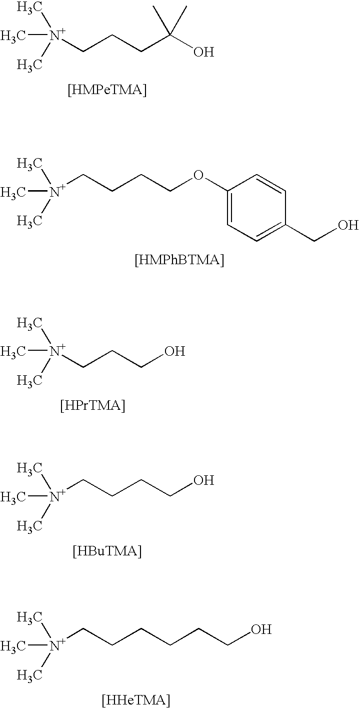

The present invention also relates to the use as defined above, for direct route peptide synthesis, comprising the use of a salt with a dedicated task of formula (I_{D}) as defined above, the cation corresponding to the following formula:

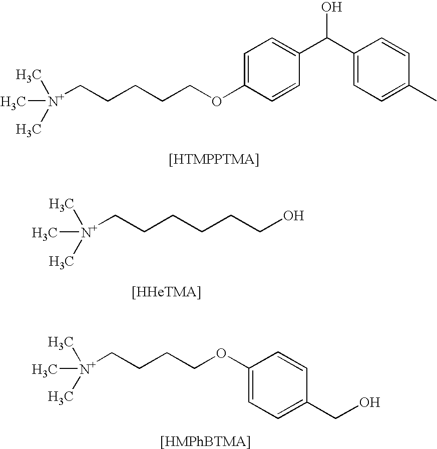

The present invention also relates to the use as defined above, for convergent peptide synthesis, comprising the use of two salts with a dedicated task of formulae (I) as defined above, the cations corresponding to the following formulae:

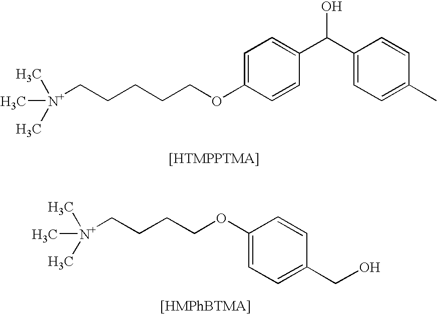

The present invention also relates to the use as defined above, characterized in that the salt with a dedicated task is:
either solubilized in a standard organic solvent such as dichloromethane, tetrahydrofuran, dioxane, acetonitrile, propionitrile, dimethylformamide, dimethylacetamide, N-methyl-pyrrolidone, acetone, toluene, chlorobenzene, dichlorobenzene, nitromethane, nitroethane, or a mixture of these solvents,
or solubilized in an ionic liquid matrix, preferably trimethylbutylammonium triflimidide or [tmba][NTf₂], 1-ethyl-3-methylimidazolium triflimidide or [emim][NTf₂], 1-butyl-3-methylimidazolium triflimidide or [bmim][NTf₂] or any other combination of onium cation and liquid anion at a temperature less than or equal to 100° C., preferably 50° C.,
or solubilized in a mixture comprising an organic solvent and an ionic liquid matrix as defined above.
or solubilized in a mixture comprising an organic solvent and a non-functionalized onium salt such as [tmba][PF₆]

According to a particular embodiment of the invention, it is possible to use different organic solvents and/or ionic liquids during peptide synthesis. It is therefore possible to envisage changing the solvent and/or ionic liquid during synthesis for example in order to obtain a better peptide coupling, improve selectivity, and improve solubility.

The use of an organic solvent/ionic liquid mixture can for example make it possible to reduce the viscosity of the reaction medium.

According to a preferred embodiment, the present invention relates to the use as defined above, for direct route peptide synthesis, characterized in that the salt with a dedicated task is in solution in an organic solvent.

Among the preferred organic solvents, there can be mentioned the aprotic dipolar solvents in general, and in particular acetonitrile, propionitrile, DMF, DMSO, DMPU, sulpholane, nitromethane, nitroethane and nitrobenzene.

The present invention also relates to the use as defined above, for direct route peptide synthesis, characterized in that the salt with a dedicated task is solubilized and immobilized in an ionic liquid matrix A₂⁺, X₂⁻,

the cation A₂⁺ being chosen from the imidazolium, pyridinium, substituted or non-substituted, ammonium, phosphonium, sulphonium cations or any other optionally functionalized onium cation, and

the anion X₂⁻ being chosen from Cl⁻, Br⁻, I⁻, F⁻, BF₄⁻, CF₃SO₃⁻, N(SO₂CF₃)₂⁻, PF₆⁻, CH₃CO₂⁻, CF₃CO₂⁻, R_{α}CO₂⁻, R_{F}CO₂⁻, RₐSO₃⁻, R_{F}SO₃⁻, R_{α}SO₄⁻, (R_{α})₃₋ₓPO₄^{x-}, x representing an integer equal to 1, 2 or 3, AlCl₄⁻, SnCl₃⁻, ZnCl₃⁻, R_{α} representing an alkyl group comprising 1 to 20 carbon atoms, R_{F} representing a perfluoroalkyl group comprising 1 to 20 carbon atoms.

Among the preferred ionic liquids, there can be mentioned [tmba][NTf₂], [emim][NTf₂], [bmina][NTf_{2], [emim][PF}₆], [bmim][PF₆], [tmba][BF₄], [emim][BF₄], [bmim][BF₄], [tmba][OTf], [emim][OTf] and [bmim][OTf].

According to a preferred embodiment, the present invention relates to the use as defined above, for reverse-route peptide synthesis, characterized in that the salt with a dedicated task is in solution in an organic solvent.

Among the preferred organic solvents, there can be mentioned the aprotic dipolar solvents in general, and in particular acetonitrile, propionitrile, DMF, DMPU, nitromethane, nitroethane and nitrobenzene.

According to a preferred embodiment, the present invention relates to the use as defined above, for reverse-route peptide synthesis, characterized in that the salt with a dedicated task is solubilized and immobilized in an ionic liquid matrix A₂⁺, X₂⁻, the cation A₂⁺ being chosen from the imidazolium, pyridinium, substituted or non-substituted, ammonium, phosphonium, sulphonium cations or any other optionally functionalized onium cation, and

the anion X₂⁻ being chosen from Cl⁻, Br⁻, I⁻, F⁻, BF₄⁻, CF₃SO₃⁻, N(SO₂CF₃)₂⁻, PF₆⁻, CH₃CO₂⁻, CF₃CO₂⁻, R_{α}CO₂⁻, R_{F}CO₂⁻, R_{α}SO₃⁻, R_{α}SO₃⁻, R_{α}SO₄⁻, (R_{α})₃₋ₓPO₄^{x-}, x representing an integer equal to 1, 2 or 3, AlCl₄⁻, SnCl₃⁻, ZnCl₃⁻, R_{α} representing an alkyl group comprising 1 to 20 carbon atoms, R_{F} representing a perfluoroalkyl group comprising 1 to 20 carbon atoms.

Among the preferred ionic liquids, there can be mentioned [tmba][NTf₂], [emim][NTf₂], [bmim][NTf₂], [emim][PF₆], [bmim][PF₆], [tmba][BF₄], [emim][BF₄], [bmim][BF₄], [tmba][OTf], [emim][OTf] and [bmim][OTf]. According to a preferred embodiment, the present invention relates to the use as defined above, for peptide synthesis by convergent route, characterized in that the salts with a dedicated task are in solution in an organic solvent.

Among the preferred organic solvents, there can be mentioned the aprotic dipolar solvents in general, and in particular acetonitrile, propionitrile, DMF, DMPU, nitromethane, nitroethane and nitrobenzene.

According to a preferred embodiment, the present invention relates to the use as defined above, for peptide synthesis by convergent route, characterized in that the salts with a dedicated task are solubilized and immobilized in an ionic liquid matrix A₂⁺, X₂⁻, the cation A₂⁺ being chosen from the imidazolium, pyridinium, substituted or non-substituted, ammonium, phosphonium, sulphonium cations or any other optionally functionalized onium cation, and

the anion X₂⁻ being chosen from Cl⁻, Br⁻, I⁻, F⁻, BF₄⁻, CF₃SO₃⁻, N(SO₂CF₃)₂⁻, PF₆⁻, CH₃CO₂⁻, CF₃CO₂⁻, R_{α}CO₂⁻, R_{F}CO₂⁻, R_{α}SO₃⁻, R_{F}SO₃⁻, R_{α}SO₄⁻, (Rα)₃₋ₓPO₄^{x-}, x representing an integer equal to 1, 2 or 3, AlCl₄⁻, SnCl₃⁻, ZnCl₃⁻, R_{α} representing an alkyl group comprising 1 to 20 carbon atoms, R_{F} representing a perfluoroalkyl group comprising 1 to 20 carbon atoms.

Among the preferred ionic liquids, there can be mentioned [tmba][NTf₂], [emim][NTf₂], [bmim][NTf₂], [emim][PF₆], [bmim][PF₆], [tmba][BF₄], [emim][BF₄], [bmim][BF₄], [tmba][OTf], [emim][OTf] and [bmim][OTf].

The present invention also relates to a peptide synthesis process by direct route (C→N) on a support as defined above, for the preparation of a peptide of the following formula (II):

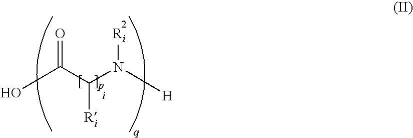

in which:
i is an integer varying from 1 to q,
q is an integer varying from 1 to 30, preferably from 1 to 20,
pᵢ is an integer varying from 1 to 20,
R′ᵢ represents an amino acid residue as defined above,
Rᵢ² represents H or a linear or branched alkyl group, comprising 1 to 20 carbon atoms and being able to form a ring with the R′ groupᵢ, the nitrogen atom carrying the group Rᵢ² and the carbon atom carrying the R′ groupᵢ, said ring comprising 3 to 20 members, in particular 5 or 6 members,

said process comprising the stages following:

a) a stage of grafting of an amino acid HOOC—[CH(R′₁)]ₚ_{₁}—N(R₁²)-GP,

R′₁, R₁² and p₁ being as defined above, and GP representing a protective group of the amine function, with the exception of Boc, in particular Fmoc, Cbz, Z, SO₂R_{g}, R_{g} representing a linear or branched alkyl group comprising 1 to 20 carbon atoms, a substituted or non-substituted aryl group, a perfluoroalkyl group comprising 1 to 20 carbon atoms,

on a soluble support of the following formula (I_{D}): A⁺-L-R—OH, X⁻, A⁺, L, R and X⁻ being as defined above,
in order to obtain the product of the following formula (II-1):

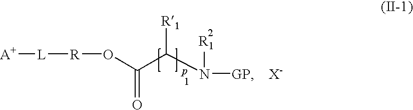

b) a stage of deprotection of the product of formula (II-1) as obtained at the end of the preceding stage in order to obtain the deprotected product of the following formula (III-1):

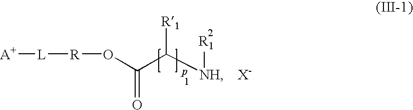

this stage of deprotection corresponding to the deprotection of the abovementioned protective group GP,

c) the sequential repetition of Stages a) and b) of grafting and of deprotection up to the obtaining of the protected supported peptide of the following formula (II-q):

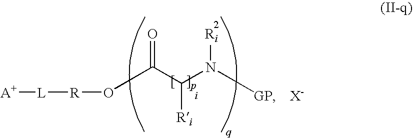

d) a stage of deprotection of the protected supported peptide of formula (II-q) as obtained at the end of the preceding stage in order to obtain the deprotected supported peptide of the following formula (III-Q):

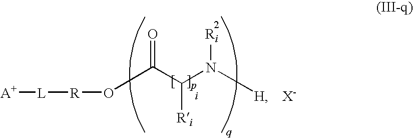

this stage of deprotection corresponding to the deprotection of the abovementioned protective group GP,

e) and a stage of cleavage from the support in order to obtain the abovementioned peptide of formula (II) and optionally to recycle the support of formula (I_{D}) A⁺-L-R—OH, X⁻,

the order of Stages d) and e) being able to be reversed.

The peptides of formula (II) can be also represented as follows:

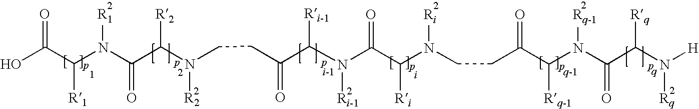

The present invention also relates to a peptide synthesis process by reverse route (N→C) on a support as defined above, for the preparation of a peptide of the following formula (IV):

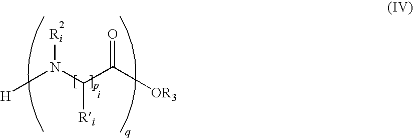

in which:
i is an integer varying from 1 to q,
q is an integer varying from 1 to 30, preferably from 1 to 20,
p, is an integer varying from 1 to 20,
R′ᵢ represents an amino acid residue as defined above,
Rᵢ² represents H or a linear or branched alkyl group, comprising 1 to 20 carbon atoms and being able to form a ring with the R′ groupᵢ, the nitrogen atom carrying the group Rᵢ² and the carbon atom carrying the R′ groupᵢ, said ring comprising 3 to 20 members, in particular 5 or 6 members,
R₃ representing a hydrogen atom or a protective group of the terminal acid function of the amino acid, and being chosen from one of the following groups: a linear or branched alkyl group, comprising 1 to 20 carbon atoms, in particular methyl or tertiobutyl, a benzyl group or an Si(ORₕ)₃ group, Rₕ representing a linear or branched alkyl group of 1 to 20 carbon atoms, and representing in particular a tertiobutyl group,

said process comprising the following stages:

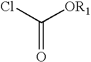

a) a stage of reaction of a compound of the following formula:
R₁ being as defined above, and representing in particular —CHCl—CCl₃ or

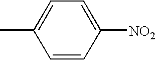

on a soluble support of the following formula (I_{D}):

A⁺-L-R—OH, X⁻

A⁺, L, R and X⁻ being as defined above,

in order to obtain a soluble support of the following formula (I_{I}):

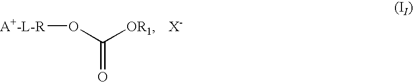

A⁺, L, R, R₁ and X⁻ being as defined above,

b) a stage of grafting of an amino acid NH(R₁²)-[CH(R′₁)]ₚ_{₁}—COOR₃, onto a soluble support of formula (I_{I}) as obtained at the end of the preceding stage,
p₁, R₁² and R′₁ being as defined above,
R₃ being as defined above,

in order to obtain a compound of the following formula (IV-1):

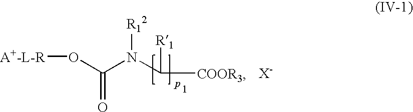

X⁻, A⁺, L, R, p₁, R′₁ and R₃ being as defined above,

c) a stage of optional deprotection of the product of formula (IV-1) as obtained at the end of the preceding stage in order to obtain the deprotected product of the following formula (V-1):

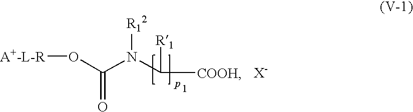

this optional stage of deprotection corresponding to the deprotection of the group R₃ when R₃ is different from H,

d) the sequential repetition of Stages b) and c) of grafting and deprotection up to the obtaining of the supported peptide of the following formula (IV-q):

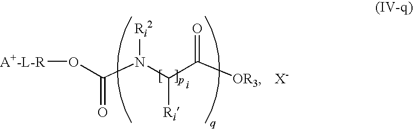

e) a stage of optional deprotection of the supported peptide of formula (IV-q) as obtained at the end of the preceding stage in order to obtain the deprotected supported peptide of the following formula (V-q):

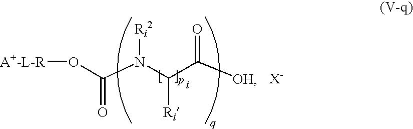

this optional stage of deprotection corresponding to the deprotection of the group R₃ when R₃ is different from H,

f) and a stage of cleavage from the support in order to obtain the abovementioned peptide of formula (IV) and optionally to recycle the support of formula (I_{D}) A⁺-L-R—OH, X⁻,

the order of Stages e) and f) being able to be reversed.

The peptides of formula (IV) can be also represented as follows:

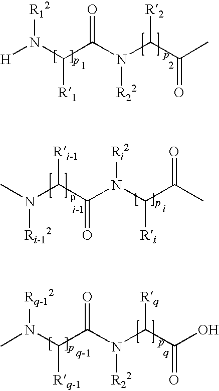

The present invention also relates to a peptide synthesis process by convergent route on a support as defined above, for the preparation of a peptide of the following formula (VI):

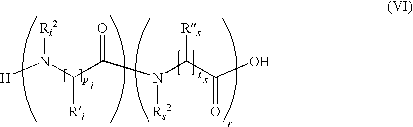

in which:
i is an integer varying from 1 to q,
q is an integer varying from 1 to 30, preferably from 1 to 20,
p, is an integer varying from 1 to 20,
R′, represents an amino acid residue,
Rᵢ² represents H or a linear or branched alkyl group, comprising 1 to 20 carbon atoms and being able to form a ring with the R′ groupᵢ, the nitrogen atom carrying the group Rᵢ² and the carbon atom carrying the R′ groupᵢ, said ring comprising 3 to 20 members, in particular 5 or 6 members,
s is an integer varying from 1 to r,
r is an integer varying from 1 to 20,
t_{S} is an integer varying from 1 to 20,
R_{S}² represents an amino acid residue,
R_{S}² represents H or a linear or branched alkyl group, comprising 1 to 20 carbon atoms and being able to form a ring with the R′ group's , the nitrogen atom carrying the group R_{S}² and the carbon atom carrying the R″_{S}, group, said ring comprising 3 to 20 members, in particular 5 or 6 members,

said process comprising the following stages:

a) the reaction of a supported peptide obtained by peptide synthesis by reverse route of the following formula (VII-I):

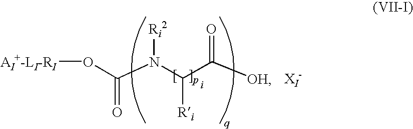

A₁⁺, L_{I}, R_{I} and X_{I}⁻ corresponding to the same definition as that given for A⁺, L, R and X⁻ above,
i, q, Rᵢ², pᵢ and R′ᵢ being as defined above,

with a supported peptide obtained by direct route synthesis of formula (VII-D):

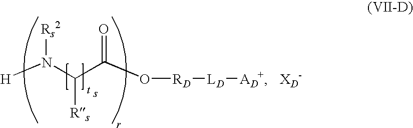

A_{D}⁺, L_{D}, R_{D} and X_{D}⁻ corresponding to the same definition as that given previously for A⁺, L, R and X⁻,
A_{D}⁺-L_{D}-R_{D} and A_{I}⁺-L_{I}-R_{I} being able to be identical or different,
and X_{D}⁻ and X_{I}⁻ being able to be identical or different,
s, r, Rₛ², t_{S} and R″_{S} being as defined above, in order to obtain a bi-supported peptide of the following formula (VIII):

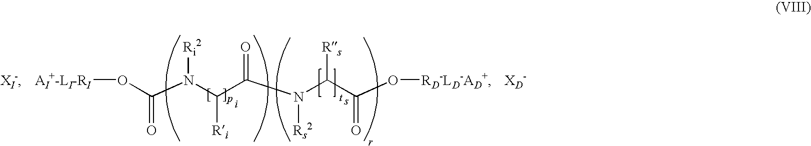

b) and a stage of cleavage of the product of formula (VIII) in order to obtain the abovementioned peptide of formula (VI), and optionally to recycle the supports of the following formula: A_{D}⁺-L_{D}-R_{D}—OH, X_{D}⁻, and A_{I}⁺-L_{I}-R_{I}—OH, X_{I}⁻.

According to a preferred embodiment, the peptide synthesis process according to the invention is characterized in that the supports are:
either solubilized in a standard organic solvent such as dichloromethane, tetrahydrofuran, dioxane, acetonitrile, propionitrile, dimethylformamide, dimethylacetamide, N-methyl-pyrrolidone, acetone, toluene, chlorobenzene, dichlorobenzene, nitromethane, nitroethane, or a mixture of these solvents,
or solubilized in an ionic liquid matrix, preferably trimethylbutylammonium triflimidide or [tmba][NTf₂], 1-ethyl-3-methylimidazolium triflimidide or [emim][NTf₂], 1-butyl-3-methylimidazolium triflimidide or [bmim][NTf₂] or any other combination of onium cation and liquid anion at a temperature less than or equal to 100° C., preferably 50° C.,
or solubilized in a mixture comprising an organic solvent and an ionic liquid matrix as defined above.

The invention also relates to a peptide synthesis process for the formulae represented above, in which the terminal acid group is esterified, in other words peptides in which the —COOH group is replaced by —COOR₃, R₃ having in particular the following meanings: a protective group of the terminal acid function of the amino acid, and being chosen from one of the following groups: a linear or branched alkyl group, comprising 1 to 20 carbon atoms, in particular methyl or tertiobutyl, a benzyl group or an Si(ORₕ)₃ group, Rₕ representing a linear or branched alkyl group of 1 to 20 carbon atoms, and representing in particular a tertiobutyl group.

The present invention also relates to compounds of the following formula (I-a):

A⁺-L-R—OW, X⁻

in which:
W represents:
   either a hydrogen atom,
   or a —COORᵢ group, R₁ representing an alkyl group comprising 1 to 20 carbon atoms or an aryl group comprising 6 to 30 carbon atoms, or a perfluoroalkyl group comprising 1 to 20 carbon atoms, said alkyl or aryl groups being optionally functionalized, R₁ representing in particular —CHCl—CCl₃ or

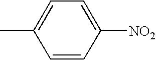

or a group of the following formula (A′):

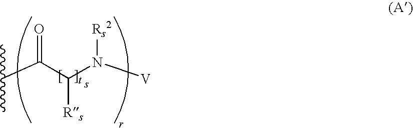

in which:
   s is an integer varying from 1 to r,
   r is an integer varying from 1 to 30, preferably from 1 to 20,
   t_{S} is an integer varying from 1 to 20,
   R″_{S} represents an amino acid residue,
R_{S}² represents H or a linear or branched alkyl group, comprising 1 to 20 carbon atoms and being able to form a ring with the R″_{S} group, the nitrogen atom carrying the group R_{S}² and the carbon atom carrying the R″_{S} group, said ring comprising 3 to 20 members, in particular 5 or 6 members,
   V represents a hydrogen atom or a protective group of the amine function, with the exception of Boc, in particular Fmoc, Cbz, Z, SO₂R_{g}, R_{g} representing a linear or branched alkyl group comprising 1 to 20 carbon atoms, a substituted or non-substituted aryl group, a perfluoroalkyl group comprising 1 to 20 carbon atoms,
   or a group of the following formula (B′):

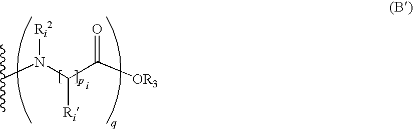

in which:
   i is an integer varying from 1 to q,
   q is an integer varying from 1 to 30, preferably from 1 to 20,
   pᵢ is an integer varying from 1 to 20,
   R′ᵢ represents an amino acid residue,
   Rᵢ² represents H or a linear or branched alkyl group, comprising 1 to 20 carbon atoms and being able to form a ring with the R′ groupᵢ, the nitrogen atom carrying the Rᵢ² group and the carbon atom carrying the R′ᵢ group, said ring comprising 3 to 20 members, in particular 5 or 6 members,
   R₃ representing a hydrogen atom or a protective group of the terminal acid function of the amino acid, and being chosen from one of the following groups: a linear or branched alkyl group, comprising 1 to 20 carbon atoms, in particular methyl or tertiobutyl, a benzyl group or an Si(ORₕ)₃ group, Rₕ representing a linear or branched alkyl group of 1 to 20 carbon atoms, and representing in particular a tertiobutyl group,
   or a group of the following formula (C′):

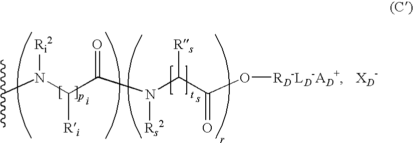

in which:
   s, r, tₛ, R″, and Rₛ² are as defined above in Formula (A′), and
   i, q, p-R′, and Rᵢ² are as defined above in Formula (B′),
   X_{D}⁻ represents a functional or non-functional anion, chosen in particular from Cl⁻, Br⁻, I⁻, BF₄, CF₃SO₃⁻, N(SO₂CF₃)₂⁻, PF₆⁻, CH₃CO₂⁻, CF₃CO₂⁻, R_{α}CO₂⁻, R_{F}CO₂⁻, R_{α}SO₃⁻, R_{F}SO₃⁻, R_{α},SO₄, (R_{α})₃₋ₓPO₄^{x-}, x representing an integer equal to 1, 2 or 3, AlCl₄⁻, SnCl₃⁻, ZnCl₃⁻, R_{α}, representing an alkyl group comprising 1 to 20 carbon atoms, R_{F} representing a perfluoroalkyl group comprising 1 to 20 carbon atoms,
   A_{D}⁺ represents a cationic entity, in particular chosen from the pyridinium, imidazolium, ammonium, phosphonium or sulphonium cations, cyclic or non-cyclic, substituted or non-substituted, and preferably ammonium or phosphonium,
   L represents an arm, in particular a linear or branched alkyl group, or aralkyl or alkaryl comprising 3 to 20 carbon atoms,
   R represents a group chosen from the following groups:
      a group of formula —C(Rₐ)(R_{b})—, Rₐ and R_{b} representing independently of one another a hydrogen atom or a linear or branched alkyl group, comprising 1 to 20 carbon atoms, the group of formula —C(Rₐ)(R_{b})— preferably representing a —CH₂—, —CH(Me)- or —C(Me)₂- group,
      a group of formula -T-Ar₁—CH(R_{c})—, in which:
         T is chosen from one of the following groups: CH₂, O, S and NR_{d}, R_{d} representing a hydrogen atom or a linear or branched alkyl group, comprising 1 to 20 carbon atoms,
         Ar₁ represents an aromatic group of the following formula:

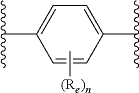

n representing an integer equal to 0, 1, 2, 3, or 4
Rₑ representing either a linear or branched alkyl group, comprising 1 to 12 carbon atoms, in particular a methyl group, or an alkoxy group comprising 1 to 12 carbon atoms, in particular a methoxy, ethoxy, propyloxy, isopropyloxy, butyloxy, isobutyloxy or tertiobutyloxy group,
R_{c} represents either a hydrogen atom, or a linear or branched alkyl group, comprising 1 to 20 carbon atoms, or an aromatic group Ar_{c} of the following formula:

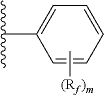

m representing an integer equal to 1, 2, 3, 4 or 5
R_{f} representing either a linear or branched alkyl group, comprising 1 to 12 carbon atoms, in particular a methyl group, or an alkoxy group comprising 1 to 12 carbon atoms, in particular a methoxy, ethoxy, propyloxy, isopropyloxy, butyloxy, isobutyloxy or tertiobutyloxy group,
A⁺, L, R and X⁻ corresponding to the same definition as that given above for A_{D}⁺, L_{D}, R_{D} and X_{D}⁻,

A_{D}⁺-L_{D}-R_{D} and A⁺-L-R being able to be identical or different,

and X_{D}⁻ and X⁻ being able to be identical or different,

the following compounds being excluded:

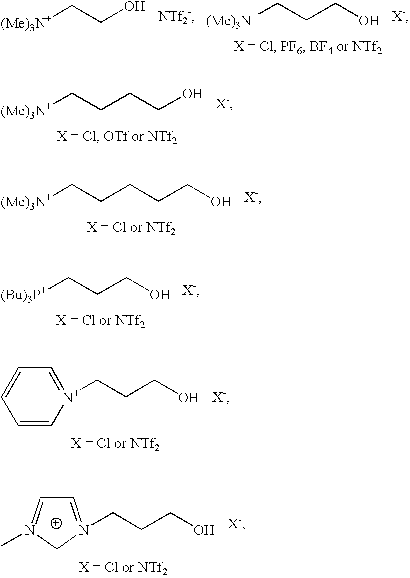

In the formula (I-a), when:

W═H, the corresponding compound is an alcohol;

W═COOR₁, the corresponding compound is an ester; W=(A′), the corresponding compound is a supported peptide (direct route)

W═(B′), the corresponding compound is a supported peptide (reverse route)

W═(C′), the corresponding compound is a bi-supported peptide (convergent synthesis)

The present invention also relates to compounds as defined above, corresponding to the following formula (I):

A⁺-L-R—OY, X⁻ (I)

in which:
A⁺, X⁻, L and R are as defined above,
Y represents:
   either a hydrogen atom, the salt of formula (I) then comprising a cation functionalized by an alcohol function and corresponding to the following formula (I_{D}): A⁺-L-R—OH, X⁻,
   or a —COORᵢ group, R₁ being as defined above, the salt of formula (I) then comprising a cation functionalized by a mixed carbonate function and corresponding to the formula (I_{I}) following:

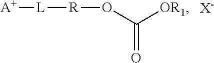

The preferred compounds according to the present invention correspond to one of the following formulae:

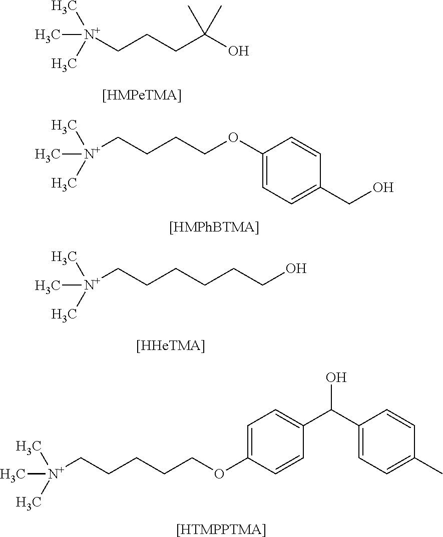

### DETAILED DESCRIPTION

### I-Synthesis of Supports

A-Primary Alcohols:
a) Single Arms with Carbonated Chains

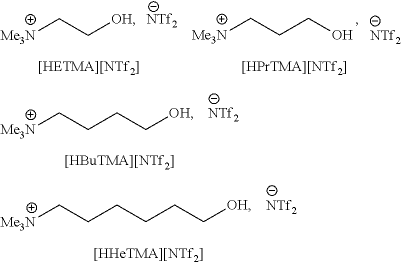

Synthesis Diagram

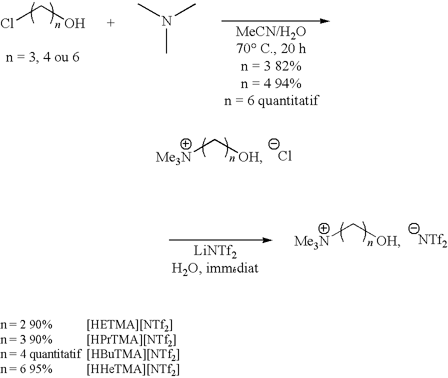

These reactions produce good results and do not produce parasitic products.

HE=hydroxyethyl; HPr=hydroxypropyl; HBu=hydroxybutyl;

HHe=hydroxyhexyl.

b) Benzyl-Type Arms (Formula (I) with X═O; Primary Benzyl Alcohol)

### Synthesis of [HMPhBTMA][NTf₂/PF₆]

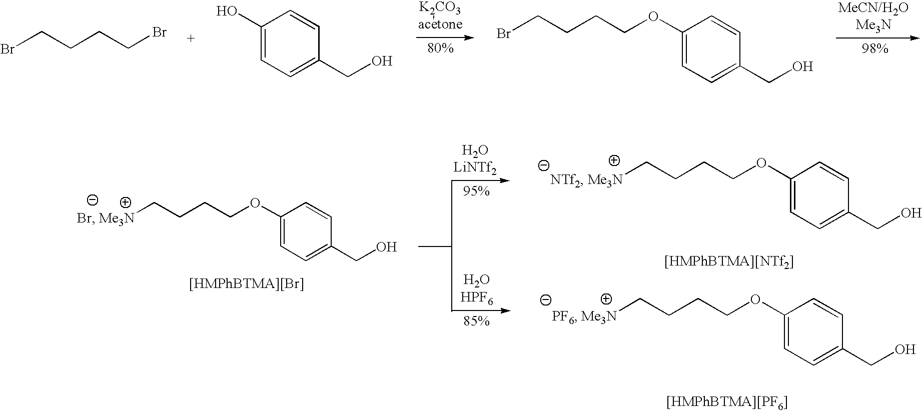

B-Secondary Alcohols:

a) Synthesis of [HPeTMA][NTf₂]

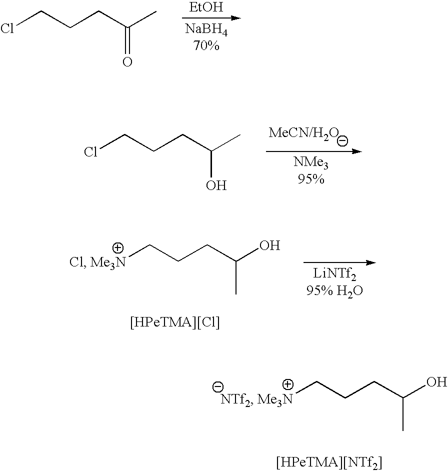

This synthesis comprises the reduction of a ketone in order to obtain a chloroalcohol, the quaternization of Me₃N and finally anion metathesis by LiNTf₂.

b) Synthesis of [HPMPTTMA][Br/NTf₂] (formula (I) with X═O; benzhydrilic alcohol)

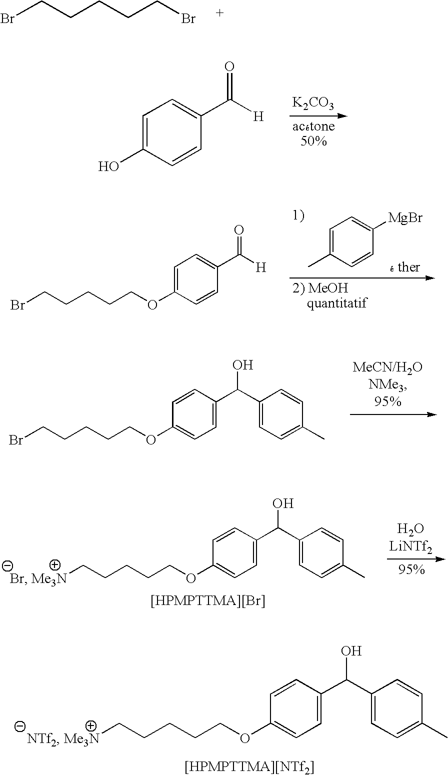

C-Tertiary Alcohols:

### Synthesis of [HMPeTMA][X]

1st stage: synthesis of the chloroalcohol precursor according to the diagram below:

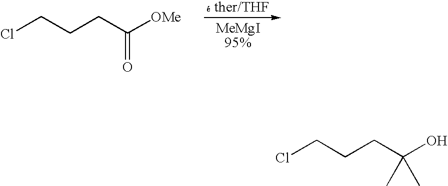

Two routes are used according to the anions aimed at:
1^{St} route: a tertiary amine is alkylated according to the following diagram:

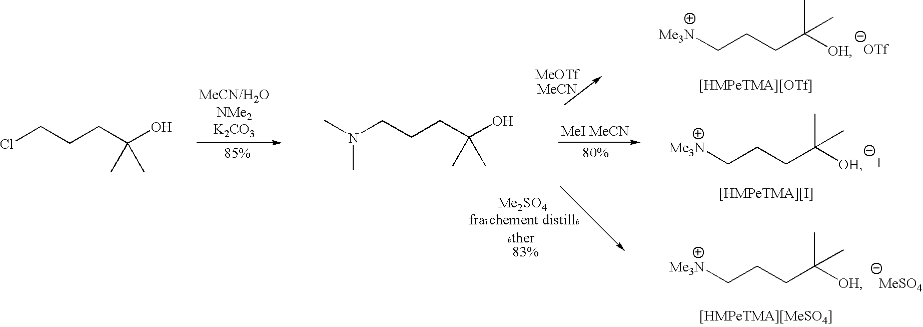

With methyl triflate, the formation of olefins by loss of water is observed.
2^{nd} route: quaternization of trimethylamine

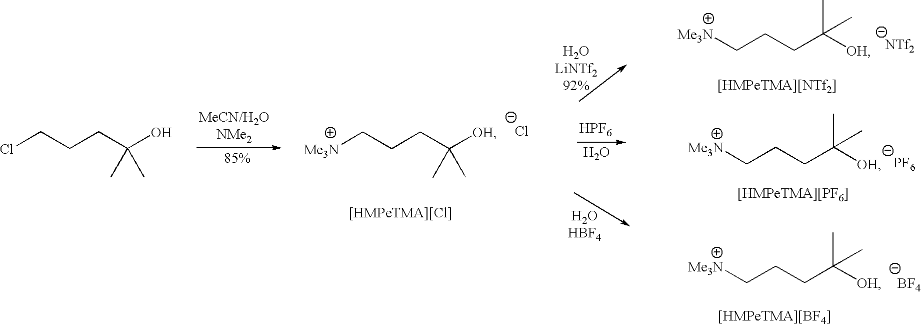

Because of the results obtained during the metathesis with HPF₆ and HBF₄, it seems preferable to carry out the metathesis with KPF₆ and NaBF₄.

### II-Reverse Route Peptide Synthesis

The principle of reverse peptide synthesis supported on onium salt with a dedicated tack is the following:

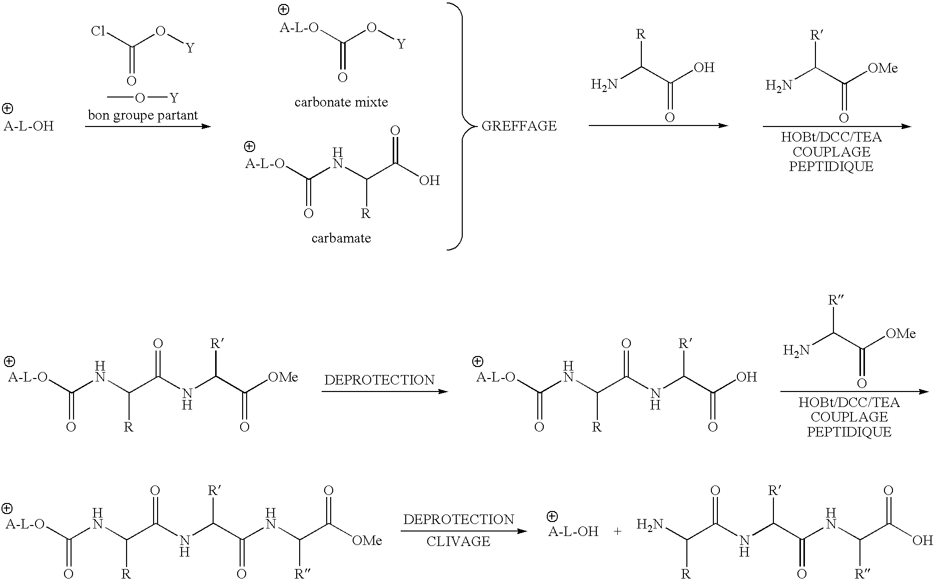

The objective is to create a ter-butyloxycarbonyl Boc group analogue, which is stable vis à vis bases, nucleophilic substances, weak acids, oxidizing agents and weak reducing agents. The salt with a dedicated task [HMPeTMA][X] was therefore used to develop the reaction conditions. The nature of the support was then diversified.

A-Study of the Formation of Mixed Carbonates with 4-Nitrophenyl Chloroformate:

a) Development of the Reaction with the Support [HMPeTMA][I]

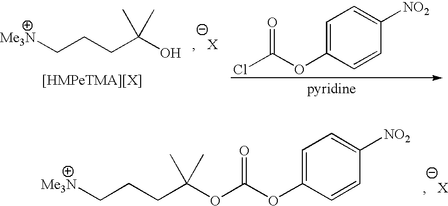

Conditions

Reaction in Acetonitrile

The reaction is carried out over 12 to 18 hours at ambient temperature with 1.9 equivalents of 4-nitrophenyl chloroformate and 3.0 equivalents of pyridine/support with X═I.

A total conversion is then observed.

But the carbonate is sensitive to water and the starting alcohol [HMPeTMA][I] is reformed by hydrolysis. This problem can easily be avoided by directly carrying out the following carbamate formation reaction (two stages in a single pot).

Reaction in Ionic Liquids

The reactions were carried out starting from the support [HMPeTMA][Cl] in solution in four equivalents of [bmim][NTf₂] or [bmim][PF₆] or [bmim][BF₄] or [bmim][OTf] by adding a few drops of acetonitrile in order to reduce the viscosity and obtain good stirring.

The carbonate is formed quantitatively in 5 to 10 hours. The conversion is therefore more rapid in the ionic liquids.

Nature of the Counter-Ion of the Salt with a Dedicated Task

By using supports [HMPeTMA][α]dried beforehand with a Kügelrohr, the formation of the carbonate is quantitative (NMR monitoring) whatever the counter-ion (X═I, Cl, BF₄, NTf₂, PF₆) whether in the acetonitrile or in the ionic liquids. The carbonates are not isolated but involved directly in the following reaction.

b) Diversification of the Nature of the Salt with a Dedicated Task

The carbonates obtained from [HPrTMA][NTf₂], [HBuTMA][NTf₂] or [HHeTMA][NTf₂], [HMPhBTMA][X](X═NTf₂ or PF₆) are formed quantitatively in 30 minutes in acetonitrile and in 15 minutes in [tmba][NTf₂].

B-Study of the Formation of the Carbamates

Grafting of the Isonipecotic Acid

a) Formation of the Carbamate [HMPeTMA-Aiso][H]

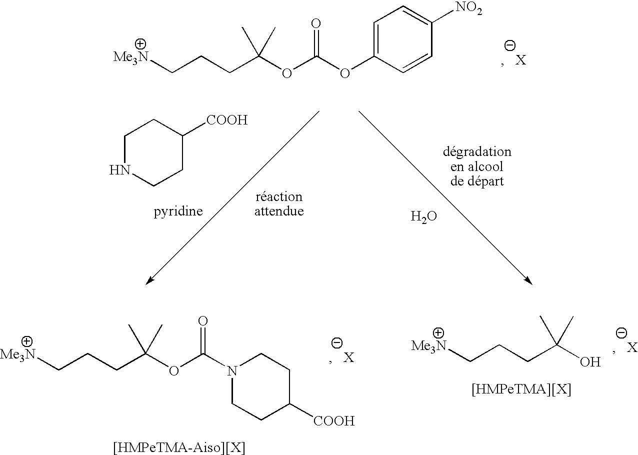

Reaction in DMF

The formation of the carbamate [HMPeTMA-Aiso][I] is carried out in DMF at ambient temperature by using 3.0 to 3.5 equivalents of isonipecotic acid and an excess of pyridine (>3 equivalents). The reaction lasts 4 to 5 days. In all cases, a mixture of carbamate [HMPeTMA-Aiso][I] (80 to 90%) and alcohol [HMPeTMA][I] (10 to 20%) resulting from the degradation of the intermediate mixed carbonate is obtained, which does not impede the remainder of the operations. The results are similar whatever the anion of the salt with a dedicated task (X═I, Cl, BF₄, NTf₂, PF₆). In all cases, the carbamate [HMPeTMA-Aiso][α] is formed (conversions of 70 to 90% in five days, the remainder of the carbonate being degraded to alcohol) under the same conditions.

Reaction in Ionic Liquids

The reactions were initiated starting from the carbonate supported in the form of a chloride in four equivalents of [bmim][NTf₂] or [bmim][PF₆] or [bmim][BF₄] or [bmim][OTf] by adding a few drops of DMF, which allow good stirring. In all cases, the reaction is slow (approximately 100 hours) and a mixture of the expected carbamate [HMPeTMA-Aiso][Cl] and alcohol [HMPeTMA][Cl] is obtained. The proportion of alcohol is similar to that obtained in DMF in the case of [bmim][NTf₂] and [bmim][OTf] (20 and 13% respectively). By contrast, the percentage of alcohol is 40% for the operations carried out in [bmim][BF₄] and [bmim][PF₆], which can probably be explained by the intrinsic presence of hydrofluoric acid and traces of water in these two ionic liquids.

b) Diversification of the Nature of the Salt with a Dedicated Task

The most hydrophobic salts with a dedicated task with the counter-ions X═PF₆ or NTf₂, were used to make it possible to carry out washings with water without loss of substrate.

When the Matrix is a Molecular Solvent

The formation of the carbamates [HPrTMA-Aiso][X], [HBuTMA-Aiso][X] and [HHeTMA][X] with X═PF₆ or NTf₂ was tested under exactly the same conditions as those relating to the synthesis of [HMPeTMA-Aiso][X], without isolation of the intermediate carbonate.

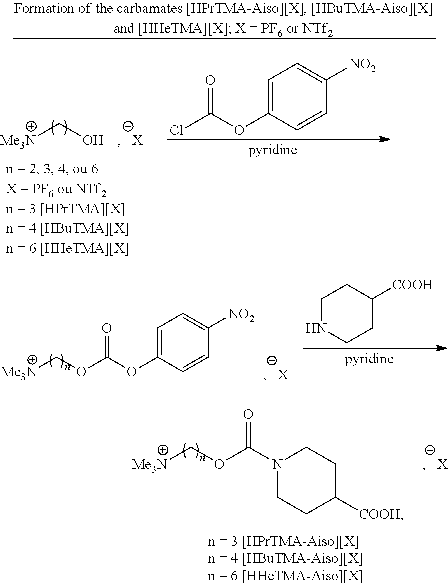

The grafting onto the salt carrying a benzyl alcohol function [HMPhBTMA][α] was carried out (diagram below). For X═NTf₂ or PF₆, the intermediate carbonate is formed in 30 minutes then the carbamate [HMPhBTMA-Aiso][X] in 18 hours. In this case, a mixture of alcohol and carbamate is also obtained in the proportions 8/92.

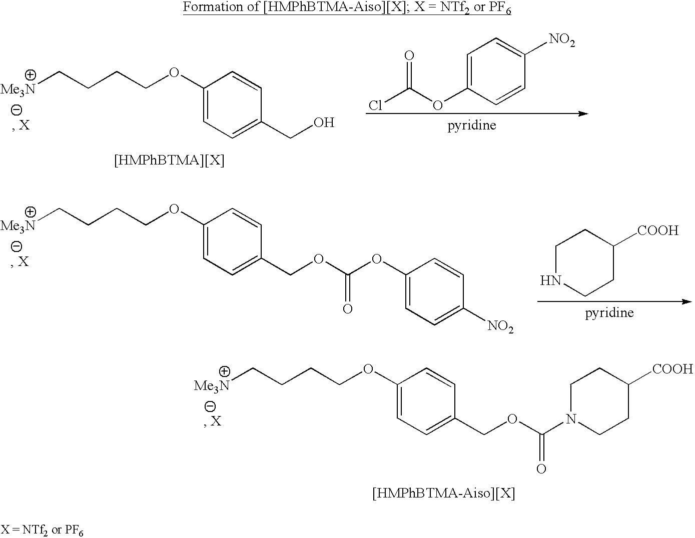

The following table shows the results with the various supports in the case where X═NTf₂. A difference in reactivity is observed between the salts carrying a primary ([HPrTMA][NTf₂], [HBuTMA][NTf₂], [HMPhBTMA][NTf₂]) or tertiary ([HMPeTMA][X]) alcohol. In the latter case, the alcohol is more hindered and the reaction times are therefore longer.

TABLE
Comparison of reaction and conversion
times with various supports.

| | | | | | Proportion of |
|---|---|---|---|---|---|
| | Duration | Duration | | Conver- | non-grafted |
| Support | Stage 1 | Stage 2 | | sion | free alcohol |
| [HPrTMA][NTf2] | 0.5 h | 18 | h | 90% | 10% |
| [HBuTMA] [NTf₂] | 0.5 h | 18 | h | 80% | 20% |
| [HHeTMA][NTf₂] | 0.5 h | 184 | h | 80% | 20% |
| [HMPeTMA][NTf₂] | 18 h | 4 | days | 85% | 15% |
| [HMPhBTMA][NTf₂] | 0.5 h | 18 | h | 92% | 8% |

When the Matrix is an Ionic Liquid

The grafting of the isonipecotic acid was carried out on the salts [HPrTMA][NTf₂] or [HBuTMA][NTf₂] in 0.95 mol/L solution in [tmba][NTf₂]. The reactions are carried out without the addition of organic solvent as the viscosity of the medium allows good stirring.

The formation of the carbonates obtained from [HPrTMA][NTf₂] or [HBuTMA][NTf₂] is carried out in 15 minutes at ambient temperature (1^{st} stage).

The carbamate [HPrTMA-Aiso][NTf₂] (2^{nd} stage) is obtained in 8 hours but a mixture of 40% alcohol [HPrTMA][NTf₂] and 60% carbamate [HPrTMA-Aiso][NTf₂] is obtained.

Similarly, the carbamate [HBuTMA-Aiso][NTf₂] (2^{nd} stage) is formed in approximately 18 hours (as in the organic solvents). A mixture of 30% alcohol [HBuTMA][NTf₂] and 70% carbamate [HBuTMA-Aiso][NTf₂] is obtained.

The ionic liquids are hygroscopic. The intermediate carbonate is not humidity-stable, which probably explains the high proportion of alcohol obtained. It would without doubt be necessary to dry these binary ionic liquids {salt with a dedicated task+ionic liquid} in order to improve the conversions. The grafting of the first amino acid was not pursued in the ionic liquids, given the difficulties encountered. We preferred to carry out this operation in a molecular solvent then dissolve these supported amino acids in the ionic liquids in order to test the peptide coupling reactions.

Grafting of natural amino acids.

Grafting of Methyl α-Amino Acids or α-Amino Esters

It is necessary to develop a generally method of grafting which is valid for all the amino acids, in particular for the α-amino acids. The salt with a dedicated task carrying a benzyl alcohol function [HMPhBTMA][PF₆] was chosen for these studies.

The conditions used for the grafting of the isonipecotic acid (Stage 1: 1.9 eq. of paranitrophenyl chloroformate; 3.0 eq. of pyridine in acetonitrile—Stage 2: 3.5 eq. of amino acid and pyridine in DMF) were tested using an α-amino acid (alanine) but the conversion of the carbamate formation stage did not exceed 40%.

The increase in the number of equivalents of alanine and/or of the reaction time in the second stage did not make it possible to improve this conversion (diagram below).

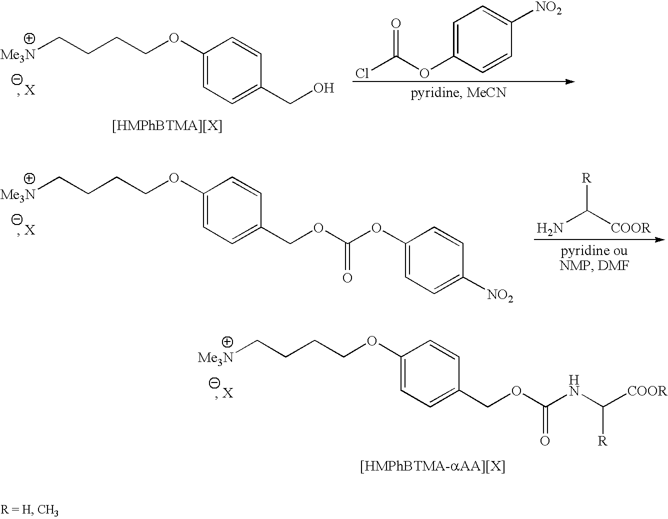

By contrast, the grafting of a β-amino acid (β-Alanine) to [HMPhBTMA][PF₆] by the method developed for isonipecotic acid is quantitative, without doubt due to the fact that the amine function is more nucleophilic than in the case of α-amino acids.

The use of N-methylmorpholine (NMP) instead of pyridine as a base allows a quantitative grafting of the isonipecotic acid in 6 hours (against 18 when pyridine is used). The major advantage provided by this base is that the grafting of methyl esters of α-amino acids is possible. Thus, the methyl esters of phenylalanine, leucine and glycine were grafted with yields of 88 to 98%. Glycine being one of the least soluble amino acids and its amine being one of the least nucleophilic, the grafting of other α-amino acids should not pose any problem.

The treatment of the reaction medium involves evaporating the DMF from the reaction medium. The residue obtained is then washed with ether then dissolved in DCM. The organic phase is then washed with water then with an aqueous solution of HPF₆ thus avoiding the problem of anion metathesis.

Grafting of Non-Methyl Amino Esters

Protective groups of the acid function other than methyl esters were envisaged. Thus, the grafting of the t-butyl ester of alanine is effective under the same conditions as those developed for the methyl amino esters with a yield of 84% of isolated [HMPhBTMA-Ala-OtBu][PF₆]. The product is contaminated by only 3% [HMPhBTMA][PF₆] (non-grafted alcohol).

The treatment developed for the reaction with methyl amino esters can be reproduced with tertio-butyl esters. In particular, the aqueous acid washings carried out during the treatment in order to eliminate the excess amino ester do not lead to cleavage of the tertiobutyl ester, although the latter is sensitive to acid conditions.

Similarly, the tri-terbutoxysilyl ester of alanine was also synthesized then grafted by analogy with Hallberg's works. The formation of the carbamate [HMPhBTMA-Ala-OSil][PF₆] is quantitative in 3 hours according to NMR ¹H. In this case, the product is not contaminated by free support [HMPhBTMA-Ala-OSil][PF₆]. The grafting is total.

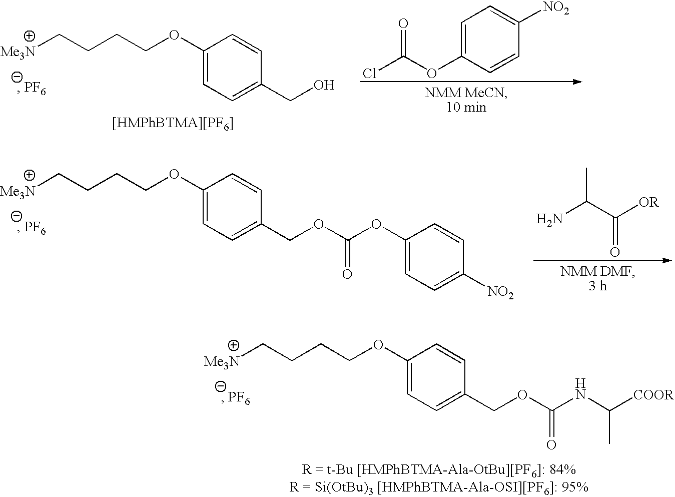

C—Peptide Coupling

Peptide coupling with an α-amino ester

The reaction of [HBuTMA-Aiso][NTf₂] and isopropylamine in acetonitrile or [TMBA][NTf₂] leads to the expected amide with 95% yield.

The coupling of [HBuTMA-Aiso][NTf₂] and of the methyl ester of glycine was carried out in CH₃CN (diagram below). The peptide bond is created quantitatively and the substitution product at the level of the carbamate function is not formed. The same good results are obtained with the supports [HMPhBTMA-Aiso][NTf₂] and [HHeTMA-Aiso][NTf₂] both in acetonitrile and in [TMBA][NTf₂]. A screening of the number of equivalents of the reagents HOBt/DCC and Gly-OMe.HCl (1.05; 1.2; 1.5 or 2.0 equivalents and double of TEA) showed that the optimum conditions are the use of 1.5 equivalents of each reagent (3.0 of TEA). The conversion then exceeds 95% (no trace of starting salt according to NMR).

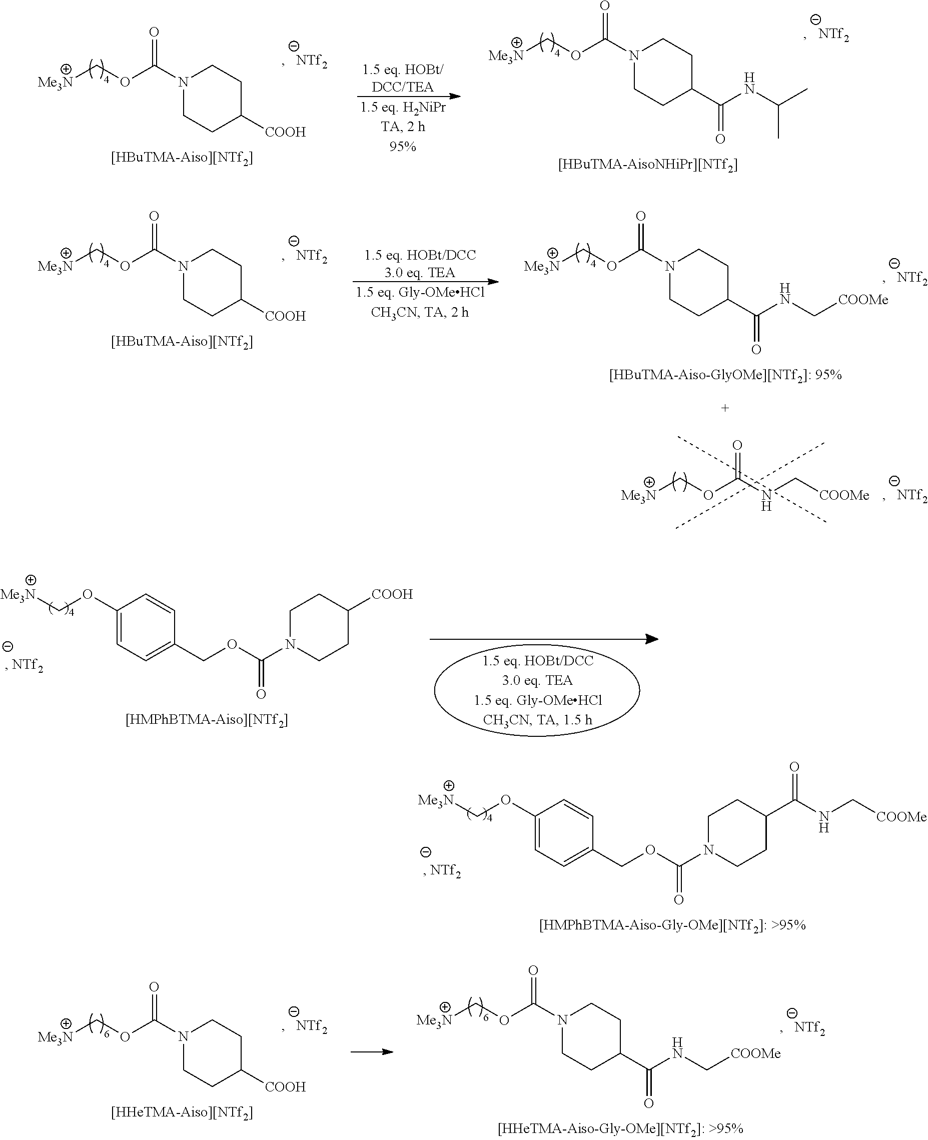

A screening of the most commonly used carbodiimides was carried out using Ala-OMe and non Gly-OMe in the coupling reaction. The conversion is quantitative with DCC, DIC EDC.HCl. We chose to pursue these studies with DCC which is the least expensive reagent. However, when the reactions are carried out on large quantities, it is preferable to use DIC the DIU urea of which is easier to eliminate than DCU.

Treatment of the Reaction

The best purification technique for the reactions carried out in acetonitrile is, after elimination of the solvent, to carry out chromatography on a column of neutral alumina with DCM as eluent which makes it possible in a first phase to eliminate any which is not attached to the onium salt with a specific task to elute the salts with a 1 to 2% DCM/MeOH mixture. The reaction was then diversified to other amino esters such as Ala-OMe, Leu-OMe, Val-OMe and Phe-OMe: [HMPhBTMA-Aiso-Ala-OMe][NTf₂], [HMPhBTMA-Aiso-Leu-OMe][NTf₂], [HMPhBTMA-Aiso-Val-OMe][NTf₂], [HMPhBTMA-Aiso-Phe-OMe][NTf₂] were obtained. The conversion is greater than 95% and the purification by chromatography on alumina proves very effective: the supported peptides are obtained with a high level of purity and can be used in the following reactions of cleavage from the support or deprotection of the acid in order to continue the peptide synthesis. The yields of pure isolated products are in the region of 65%.

Another alternative involves changing the counter-ion of the onium salt support by substituting the ⁻NTf₂ by a ⁻PF₆ ion (use of [HMPhBTMA][PF₆] instead of [HMPhBTMA][NTf₂]). It is then possible to carry out aqueous acid washings with solutions of HPF₆ (more problems with metathesis, the counter-ion of the washing solution and the ammonium salt being the same) and to more easily eliminate AA-OMe: the aqueous washings with HPF₆ leading to the formation of [H₃N-AA-OMe][PF₆]. PF₆ being less lipophilic than NTf₂, this species passes into the aqueous phase.

The novel treatment therefore involves a filtration of the reaction medium. The acetonitrile of the filtrate is then evaporated. The residue is dissolved in DCM and this phase is washed three times with water, then three times with an aqueous solution of HPF₆ (1<pH<2). The organic phase is dried over Na₂SO₄, filtered and the DCM is evaporated. The residue is then washed with ether. The yield is approximately 85% for a supported dipeptide (against 65% when the counter-ion is NTf₂ after purification on an alumina column). [HMPhBTMA-Aiso-Ala-OMe][PF₆] and [HMPhBTMA-Aiso-Leu-OMe][PF₆] were synthesized by following this protocol.

Apart from the fact that the use of the salt [HMPhBTMA][PF₆] is associated with a more easily automatable treatment, the cost of the ammonium salts comprising ⁻PF₆ as anion is lower that those comprising an ⁻NTf₂ (LiNTf₂ much more expensive than KPF₆).

Deprotection of the Terminal Acid Function

The stage of deprotection of the terminal acid function occurs:
either at the supported dipeptide stage when the first grafted amino acid is isonipecotic acid, and the second is an α-amino ester;
or just after the grafting if the acid function of the first grafted amino acid is protected.

a) Case of the Methyl Esters

The reaction of the methyl esters with excess potassium trimethylsilanolate leads to the potassium salts of the corresponding carboxylic acids. In the case of dipeptides starting from isonipecotic acid, the yields are quantitative: no cleavage is observed at the level of the carbamate. In the case where the first grafting is carried out with a natural amino ester, a partial cleavage of 5 to 10% is observed at the level of the carbamate function thus releasing the starting support. The conditions (reaction time, number of equivalents of Me₃SiOK, drying of the support) were modified but without improvement. This cleavage causes a drop in yield. However, a simple filtration on celite is sufficient to eliminate the substrates not attached to the support and makes it possible to continue the peptide synthesis under good conditions.

The dipeptides [HMPhBTMA-Aiso-Leu-OK][PF₆], [HMPhBTMA-Aiso-Phe-OK][NTf₂] and [HMPhBTMA-Aiso-Val-OK][NTf₂] the terminal acid function of which is deprotected were obtained. The supported deprotected amino acids [HMPhBTMA-Leu-OK][PF₆] and [HMPhBTMA-Gly-OK][PF₆] were also synthesized.

b) Case of the Other Esters

The methyl esters are cleaved under relatively severe conditions (Me₃SiOK) which promote racemization. This is why the use of other esters was envisaged. The cleavage of [HMPhBTMA-Ala-OtBu][PF₆] both using aqueous or anhydrous HCl or HPF₆ leads to a partial or total cleavage of the carbamate bond.

The use of supported tri-terbutoxysilyl α-amino esters was then envisaged, but the cleavage of the ester also leads to cleavage from the support under the tested conditions (conditions 1: aqueous solution of HPF₆ to 60%/MeCN: 5/95, 20 minutes at ambient temperature; conditions 2: 0.2 equivalent of HPF₆ with respect to the support, 20 minutes at ambient temperature). Hallberg used the TFA in order to deprotect the terminal acid function, but the use of this reagent was not envisaged as the carbamate bond of the grafting is cleaved under these conditions.

Continuation of the Peptide Synthesis

The dipeptides [HMPhBTMA-Leu-Ala-OMe][PF₆] and [HMPhBTMA-Gly-Ala-OMe][PF₆] were synthesized according to the following diagram:

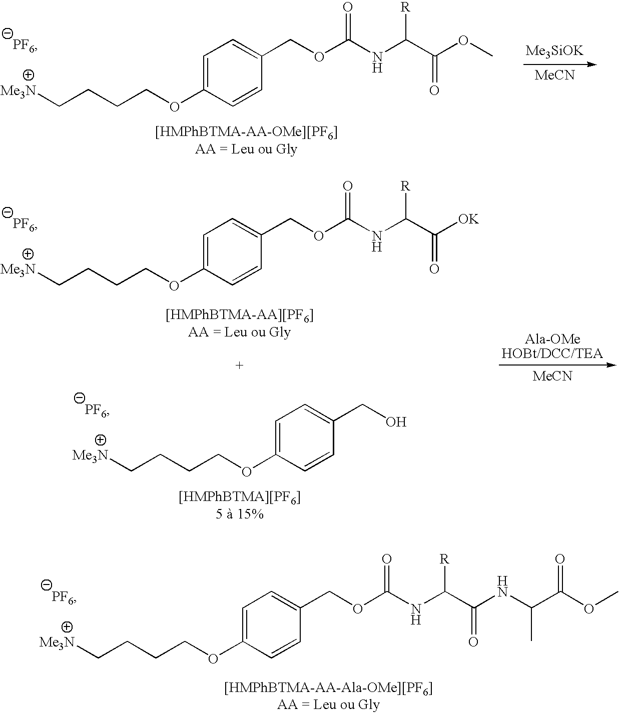

In the case where the first amino acid grafted is isonipecotic acid, the stages of grafting, peptide coupling and cleavage of the protective group of the terminal acid function are perfected, and the synthesis can therefore be continued (see diagram below). The tripeptides [HMPhBTMA-Aiso-Leu-Gly-OMe][PF₆], [HMPhBTMA-Aiso-Leu-Phe-OMe][PF₆], [HMPhBTMA-Aiso-Leu-Val-OMe][PF₆], [HMPhBTMA-Aiso-Phe-Leu-OMe][NTf₂] were thus synthesized.

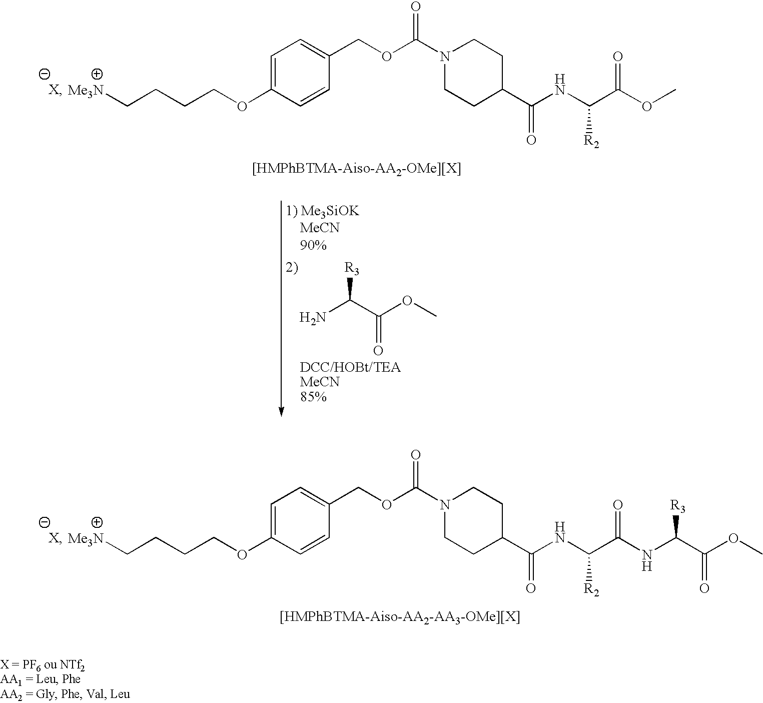

### Synthesis of Supported Tripeptides

Cleavage from the Support

[HMPeTMA-Aiso-NHBn][I] is cleaved quantitatively in 2.5 hours by a TFA/DCM: 1/1 mixture as follows:

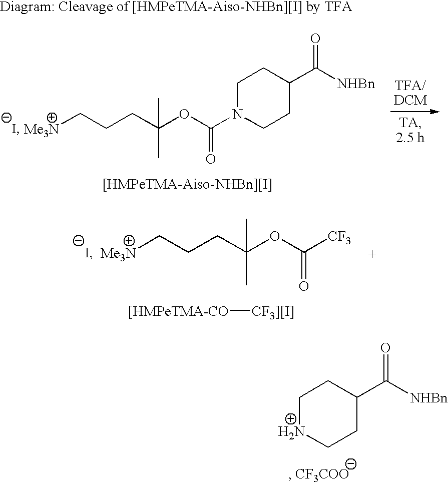

The carbamate of [HBuTMA-Aiso-NHiPr][NTf₂] is not cleaved in an acid medium, either by a 12N HCl aqueous solution, or by a TFA/DCM mixture: in 24 hours at ambient temperature, only 10% of the product reacts in order to produce the free peptide and the corresponding trifluoroacetate. The use of five equivalents of Me₃SiI relative to [HBuTMA-Aiso-NHiPr][NTf₂] makes it possible to cleave the support (see diagram below). The reaction is terminated after four hours in acetonitrile at 50° C. The reaction medium is then added to four equivalents of MeOH. After evaporation of the solvents, the addition of DCM and water to the residue makes it possible to separate the salt from the peptide. The amide bond is not cleaved under these conditions.

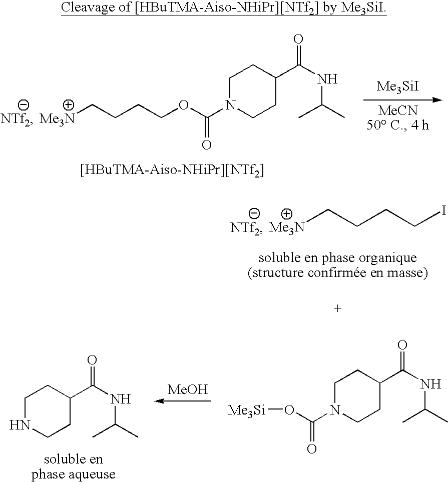

The cleavage of the carbamate of [HMPhBTMA-Aiso-AA-OMe][NTf₂] was successfully carried out by TFA. The conditions of the reaction are optimum for 10 equivalents of TFA with respect to the support in acetonitrile in 10 to 20% solution. The reaction takes 10 minutes at ambient temperature. After evaporation of the solvents, a mixture of water and DCM is added to the residue. The peptide released is solubilized in aqueous phase since the support, in the form of trifluoroacetic ester, remains in organic phase (see diagram below). The crude product yield is 95%. The peptides Aiso-Leu-OMe, Aiso-Phe-OMe and Aiso-Val-OMe were thus isolated.

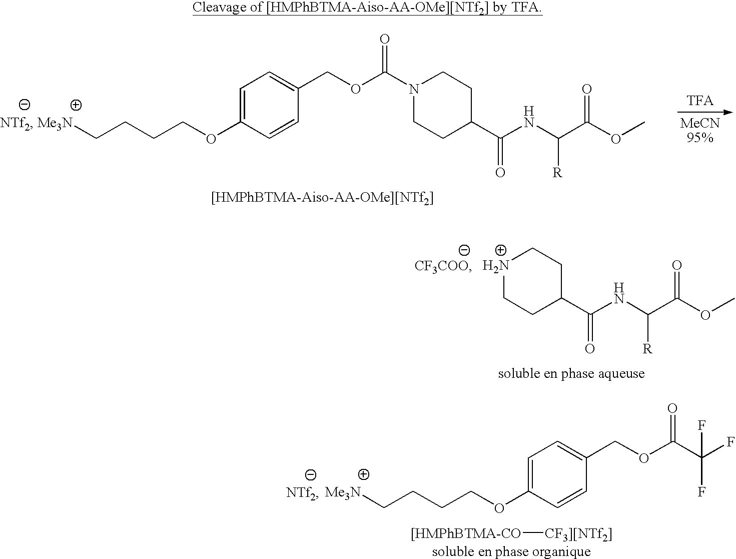

The cleavage of [HMPhBTMA-Aiso][PF₆] with 1.5 equivalents of TMSBr in acetonitrile is quantitative in 30 minutes. It is then sufficient to evaporate the solvent and to add DCM and water to the residue in order to separate the peptide from the support. The gross yield is close to 95%. The support [HMPhBTMA][PF₆] is not regenerated under these conditions.

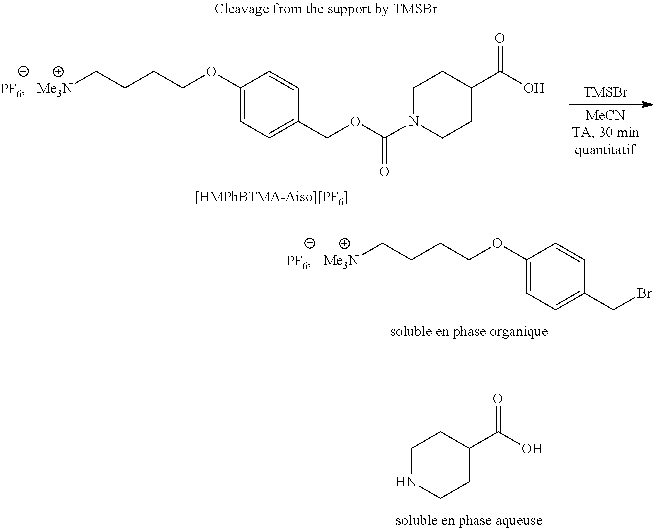

### III-PEPTIDE SYNTHESIS BY DIRECT ROUTE

The objective is to test the feasibility of supported peptide synthesis on ionic liquid or onium salt with a specific task by grafting the amino acid by its acid function to the support and by carrying out the coupling reactions on the amine function thus supported. The synthesis was envisaged with the Fmoc strategy which is the most commonly used.

The principle of direct peptide synthesis supported on onium salt with a specific task is the following:

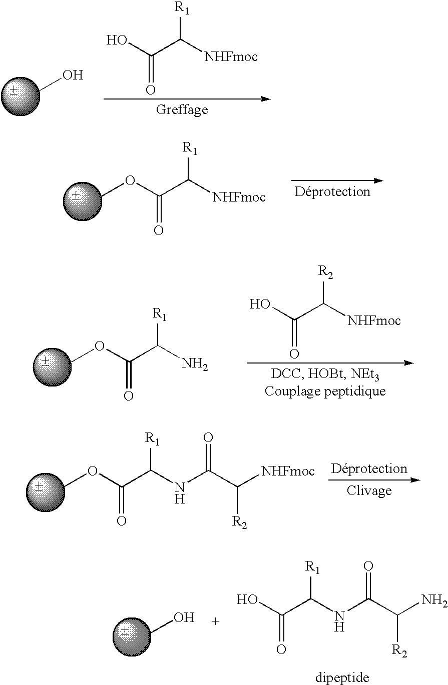

In this diagram,

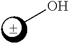

represents either a binary ionic liquid, i.e. a solution of an onium salt with a specific task carrying a hydroxyl function in an ionic liquid matrix, or a solution of an onium salt with a specific task carrying a hydroxyl function in a molecular solvent.

A first amino acid is grafted onto the support by esterification. The terminal amine function is then deprotected before being involved in the peptide coupling reaction with a second amino acid. After deprotection, a last cleavage stage makes it possible to release the peptide formed and to regenerate the support.

Three generations of supports were studied. The structure of the support was modified and optimized so that the ester bond serving for the grafting is stable under the conditions of the syntheses and the treatments of the reaction media.

A-Supports:

The salts with a dedicated task [HHeTMA][NTf₂] and [HMPhBTMA][NTf₂] were used.

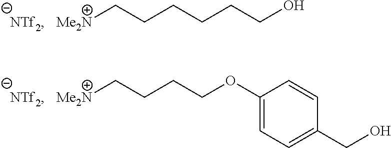

The esterification reactions of these two supports in the presence of 1.5 equivalents of DCC; 0.1 of DMAP and 1.1 of Fmoc-alanine in acetonitrile are quantitative according to NMR monitoring. The yield after treatment is close to 90%. The diagram below represents the esterification between [HHeTMA][NTf₂] or [HMPhBTMA][NTf₂] and Fmoc-alanine.

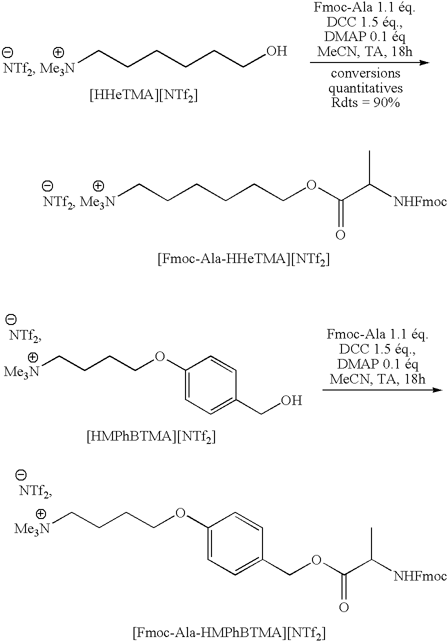

The treatment is easy: the majority of the urea is eliminated by filtration. The remaining traces of urea and the excess amino acid are eliminated by washings with ether. The supported amino acids [FmocAla-HHeTMA][NTf₂] and [FmocAla-HMPhBTMA][NTf₂] are then dissolved in DCM then extracted by two times one-tenth by volume of 1N aqueous solution of HCl, which eliminates the remaining traces of DMAP.

No problem of stability of the products was observed (no cleavage of the ester nor of the protective group of the terminal amine).

B-Deprotection of the Terminal Amine Function:

The Fmoc group is cleaved by a 1/5 piperidine/DMF mixture in 15 minutes. The deprotection of [FmocAla-HHeTMA][NTf₂] and [FmocAla-HMPhBTMA][NTf₂] is effective in anhydrous acetonitrile. The treatment involves evaporating the solvent then extracting the residue obtained with ether in order to eliminate the products of degradation of the Fmoc. The yield is greater than 90%. This stage of deprotection of the terminal amine function of [FmocAla-HHeTMA][NTf₂] or [FmocAla-HMPhBTMA][NTf₂] is represented as follows:

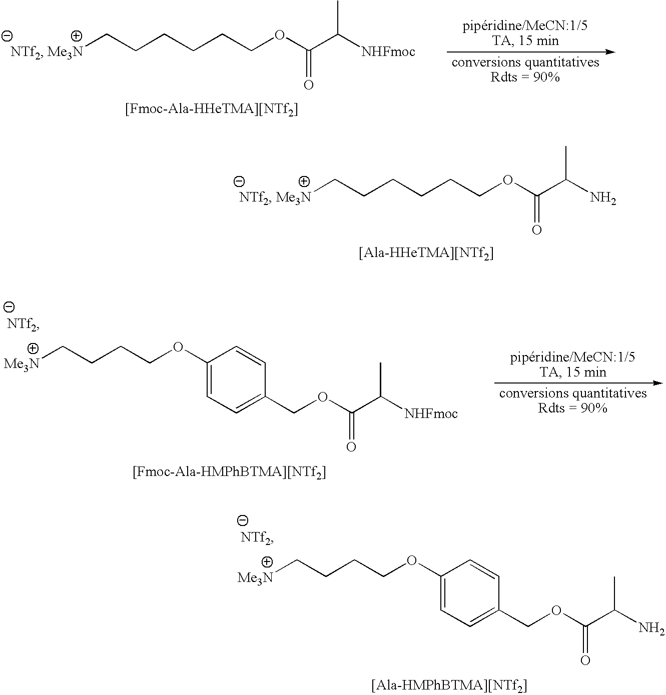

The cleavage of the ester function does not take place during the deprotection, which confirms that the supports used are stable under the conditions implemented.

C-Peptide Coupling:

The Fmoc-leucine was selected for the study of the peptide coupling as this amino acid (as well as the Fmoc-alanine) is that which poses fewer problems during the reaction (excellent yields, no protection of the side chain, less formation of dicetopiperazine compared with glycine and proline). The standard reaction conditions on solid support were applied (1.5 equivalents of DCC, HOBt, TEA and of Fmoc-leucine in a DCM/DMF: mixture 1/1, reaction for two hours at ambient temperature) in acetonitrile. The conversion is total according to NMR. The peptide coupling stage between [Ala-HHeTMA][NTf₂] or [Ala-HMPhBTMA][NTf₂] and Fmoc-leucine is represented as follows:

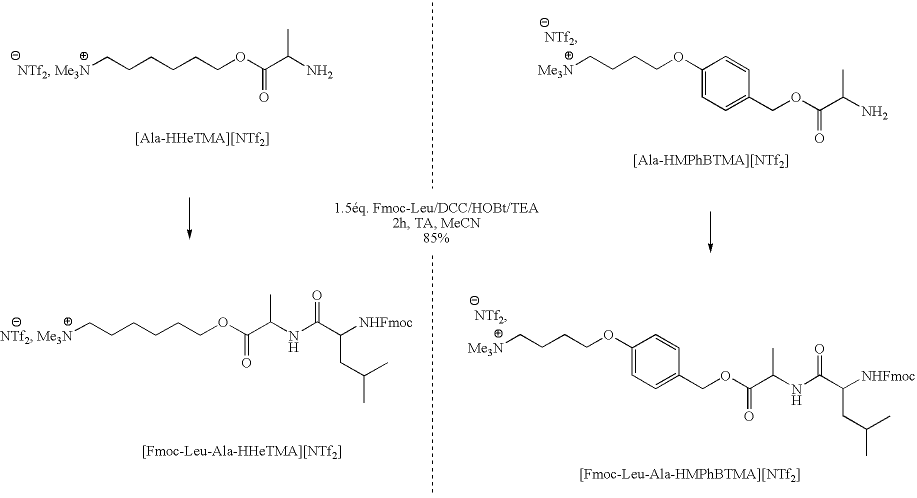

The treatment of the reactions was optimized in a similar fashion to the studies for reverse-route peptide synthesis. After filtration and evaporation of the acetonitrile, the residue is dissolved in DCM and this phase is washed with an aqueous solution of hydrochloric acid in order to eliminate [HNEt₃][OBt]. In the case where the coupling reaction was not total, this washing also has the advantage of eliminating the starting product [Ala-HHeTMA][NTf₂] or [Ala-HMPhBTMA][NTf₂]: in fact the salts having a protonated free amine pass into aqueous acid phase, since the expected product the terminal amine of which is protected by a Fmoc group remains in organic phase. The DCU and the excess Fmoc-leucine are then eliminated by washings with ether. The yields are greater than 85%.

D-Deprotection of the Terminal Amine Function:

The deprotection of [Fmoc-Leu-Ala-HMPhBTMA][NTf2] by piperidine is effective but 15% of support cleavage products are observed. NMR monitoring using benzyl —CH₂— shows the presence of the supported alcohol [HMPhBTMA][NTf₂].

The cleavage by formation of dicetopiperazine at the deprotected supported dipeptide stage is a recurrent problem observed during peptide synthesis by Fmoc technology on Wang resin (analogous to [HMPhBTMA][NTf₂]). The cleavage observed is due to the same phenomenon. This reaction involves the nucleophilic attack of the amine terminal on the ester function serving for the grafting (see diagram below). It causes not only a drop in the yield of the synthesis, but also the appearance of peptide sequences comprising the deletions of amino acids by grafting onto the support which was regenerated.

The diagram below represents the formation mechanism of dicetopiperazine DKP.

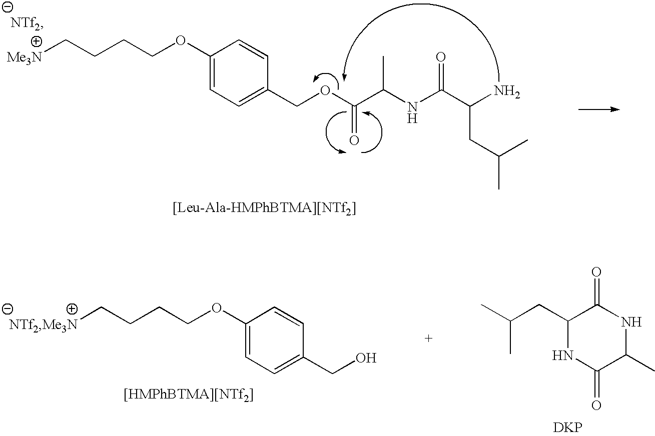

The same results can be observed for the deprotection of [Fmoc-Leu-Ala-HHeTMA][NTf₂].

Reactions in Ionic Liquids

The feasibility of the reactions in ionic liquids (deprotection of [Fmoc-Ala-HMPhBTMA][NTf₂], peptide coupling with Fmoc-leucine, deprotection of [Fmoc-Leu-Ala-HMPhBTMA][NTf₂]) was also tested with the support {[HMPhBTMA][NTf₂]/four equivalents of ionic liquid [tmba][NTf₂]} retaining the same experimental protocols (addition of acetonitrile in order to guarantee good stirring, identical treatments). The yields are comparable to those observed for the operations in standard organic solvents.

The monitoring of the reactions with the benzylated support [HMPhBTMA][NTf₂] is easy as the support absorbs UV. The retention time of the supported peptides non-protected by the Fmoc group are less than those of the protected peptides: the technique therefore seems adequate for monitoring the reactions of coupling and deprotection of the Fmoc group.

E-Development of another Support [CTMPTTMA][NTf₂]:

The preceding works led us to study the support [CTMPTTMA][NTf₂] represented below:

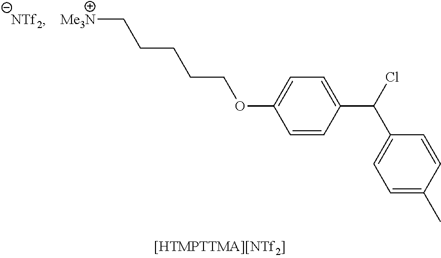

The objective was to create a salt with a dedicated task (by analogy with the existing solid supports) for which the cleavage by formation of DKP at the deprotected supported dipeptide stage is negligible.

Under the reaction and treatment conditions developed for the synthesis on onium salt, the support must be insoluble in water (DCM/water extractions); stable in aqueous acid medium (aqueous acid washings after the peptide coupling reactions) and stable in basic medium (use of piperidine, TEA, DMAP).

a) Grafting of the First Amino Acid

The grafting of the first amino acid is carried out in several stages.

The alcohol at the benzhydryl position of [HTMPPTMA][Br] is substituted quantitatively by a chlorine by reaction with 1.5 equivalents of thionyl chloride over 20 minutes in anhydrous acetonitrile. The Fmoc-amino acid is grafted by esterification over 30 minutes:

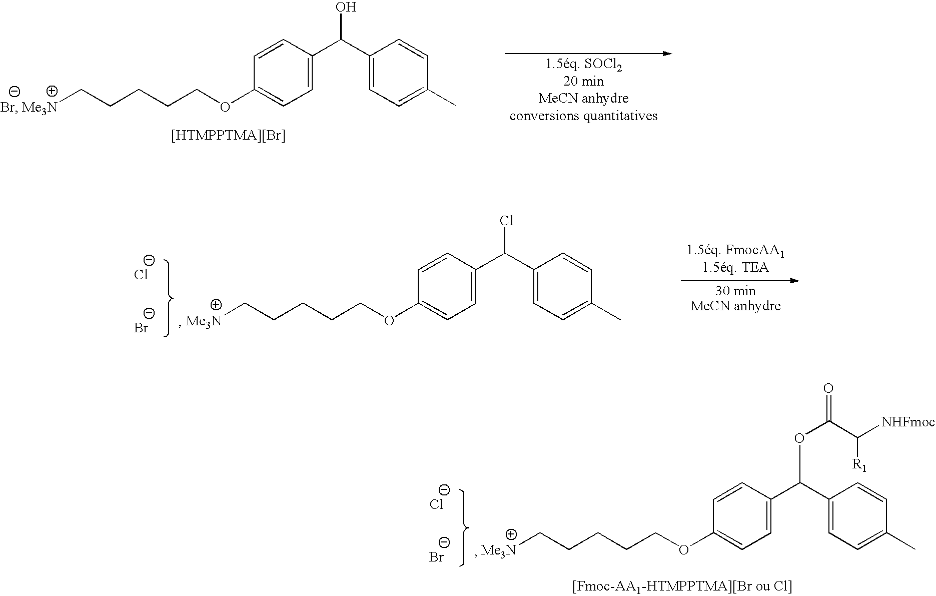

The counter-ion of the support is either a bromide (initial anion of the onium salt), or a chloride (metathesis during the chlorination stage). The experiment shows that [Fmoc-AA₁-HTMPTTMA][Br or Cl] is not soluble in the water, which is essential for the treatments developed previously. A metathesis reaction of the counter-ion has even so been envisaged, on the one hand in order to know the exact nature of this anion, on the other hand in order to avoid retaining counter-ions with a nucleophilic character which could be at the origin of secondary reactions. The hexafluorophosphate anion was chosen since it is possible to carry out washings with an aqueous solution of HPF₆ without risking anion exchange reactions. A metathesis of the counter-ion is then carried out by KPF6 over two hours in acetonitrile:

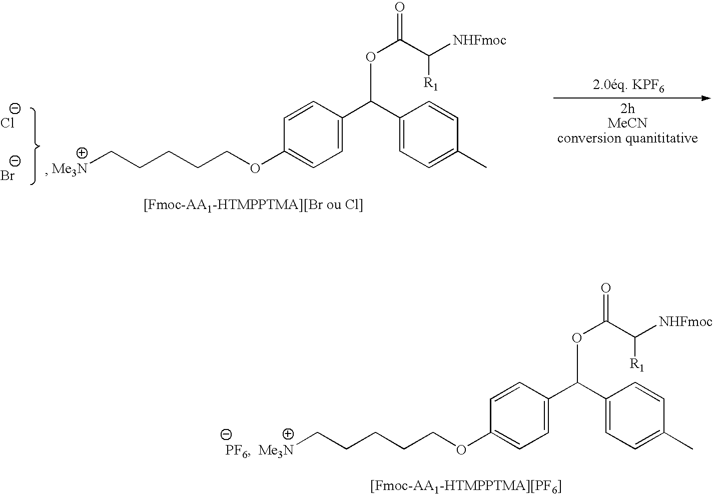

The terminal amine function can then be deprotected by piperidine under the same conditions as those developed for the other salts with a dedicated task:

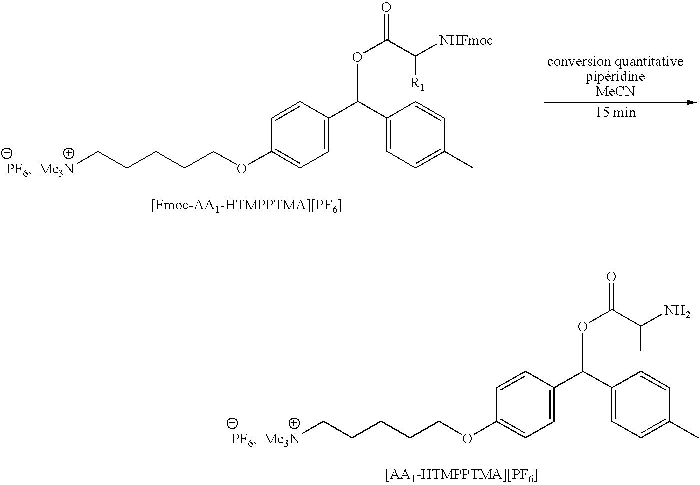

The average yield over these four stages is approximately 85%. The grafting level is quantitative: No free [HTMPTTMA][PF₆] remains. [Ala-HTMPPTMA][PF₆], [Gly-HTMPPTMA][PF₆], [Ile-HTMPPTMA][PF₆], [Leu-HTMPPTMA][PF₆], [Phe-HTMPPTMA][PF₆] and [Val-HTMPPTMA][PF₆] were thus synthesized.

b) Peptide Coupling

The peptide coupling was tested (1.5 eq. of TEA, of Fmoc-amino acid, of HOBt and of DCC (or DIC)) and is quantitative:

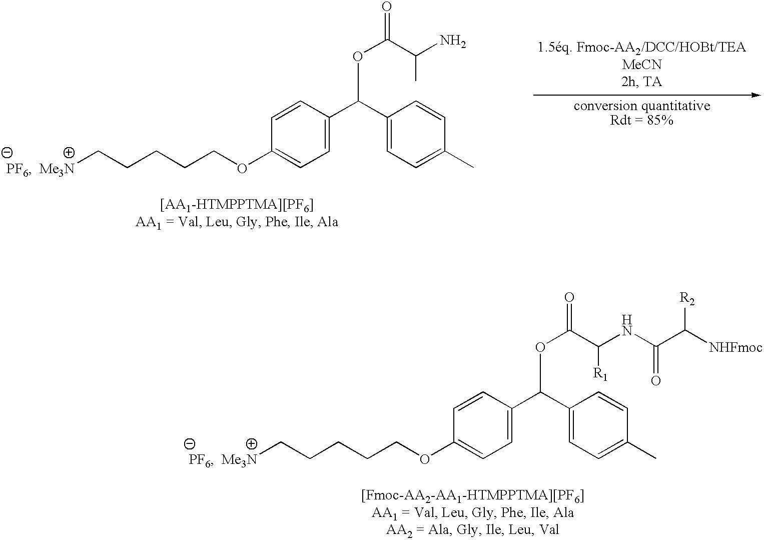

The treatment is the same as that developed for the reverse route: The reaction medium is filtered. After evaporation of the acetonitrile, the residue is dissolved in DCM. This phase is washed with water then with an aqueous solution of HPF₆. After drying and evaporation, the residue is then washed with ether. [Fmoc-Ala-Ile-HTMPPTMA][PF₆], [Fmoc-Ala-Phe-HTMPPTMA][PF₆], [Fmoc-Ala-Val-HTMPPTMA][PF₆], [Fmoc-Gly-Leu-HTMPPTMA][PF₆], [Fmoc-Gly-Phe-HTMPPTMA][PF₆], [Fmoc-Gly-Val-HTMPPTMA][PF₆], [Fmoc-Ile-Leu-HTMPPTMA][PF₆], [Fmoc-Leu-Ala-HTMPPTMA][PF₆] and [Fmoc-Val-Ile-HTMPPTMA][PF₆] were thus synthesized. The yield of isolated product is of the order of 85%.

No cleavage from the support is observed during the aqueous acid washings (benzhydryl sensitive to acid conditions), probably due to the biphasic medium.

The coupling methods using the carbodiimides (DCC, DIC or EDCI) and HOBt were applied successfully. These reagents were chosen as they are commonly used and they are not salts. These studies have led to final use of the supports of ammonium salts with a ⁻PF₆ counter ion. Numerous reagents in the form of a salt comprising this same counter-ion exist in the literature. Their use will not therefore lead to any undesirable metathesis reaction.

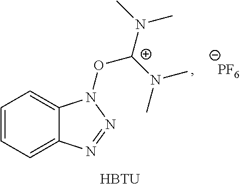

The coupling reagent HBTU, very often used in peptide synthesis, was therefore used (1.5 equivalents, all other conditions moreover retained) successfully. The elimination of the excess reagent and degradation products is total during the treatment (washings with ether and aqueous acid extraction), and is even easier than the total elimination of the ureas originating from the carbodiimides (DIU, DCU) by the preceding method, in particular for the syntheses on large quantities. The technology described here can therefore be adapted to other coupling methods, in particular to all the reagents in the form of salt with a ⁻PF₆ counter ion (BOP, PyBOP, PyBroP, HATU, HAPyU, HAPipU . . . ).

The peptide reaction coupling time is 30 minutes, and the coupling reaction conversions are always quantitative.

c) Deprotection of the Amine Function and Cleavage by Formation of DKP

The following stage is the deprotection of the terminal amine function. In order to minimize the formation of dicetopiperazine, it is necessary to minimize the life of the deprotected supported dipeptide and involve it as rapidly as possible in the following peptide coupling reaction.

The Fmoc group is cleaved by a 1/5 MeCN/piperidine mixture, followed by washings with an aqueous solution of HPF₆: 5% DKP is obtained.

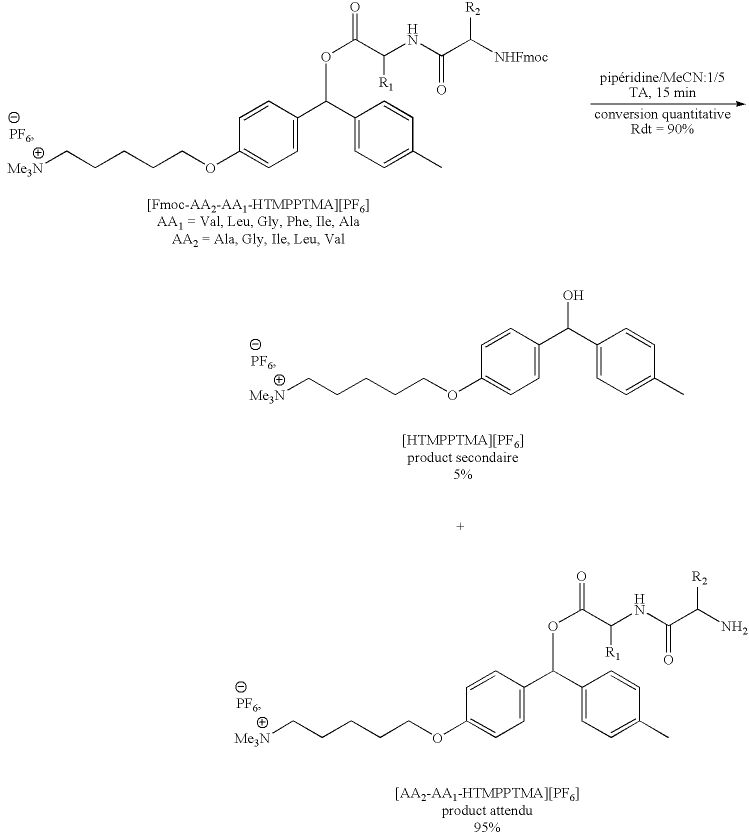

In fact, the latter cause the protonation of the amine terminal which is therefore more nucleophilic and can no longer attack the ester function serving for the grafting. These washings are possible as [H₃N-AA₂-AA₁-HTMPPTMA]([PF₆])₂ is more soluble in dichloromethane than in aqueous phase, as the spacer arm of the onium salt is lipophilic. The formation of DKP is optimized with respect to the salt [Leu-Ala-HMPhBTMA][NTf₂] (15% cleavage).

d) Continuation of the Peptide Synthesis

Peptide counting with a third Fmoc-amino acid was carried out:

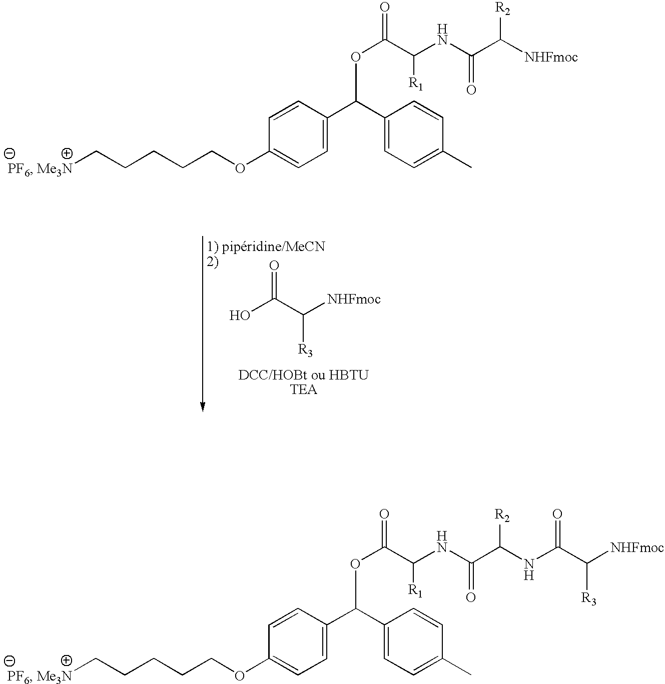

[Fmoc-Gly-Ala-Phe-HTMPPTMA][PF₆], [Fmoc-Leu-Ala-Phe-HTMPPTMA][PF₆], [Fmoc-Val-Gly-Phe-HTMPPTMA][PF₆] and [Fmoc-Val-Leu-Ala-HTMPPTMA][PF₆] were thus synthesized. The NMR¹H spectrum at 300 MHz in acetone d₆ is given below.

e) Cleavage from the Support

Cleavage was developed on [Ala-HTMPTTMA][PF₆] and [Val-Leu-Ala-HTMPTTMA][PF₆]. The supported peptide is solubilized in methanol and 0.01 eq. of an aqueous solution of HPF₆ is added. The mixture is taken to reflux for one hour. The cleavage is quantitative under these conditions. The methanol is then evaporated off. DCM and water are then added to the residue. The amino acid or the released peptide is soluble in aqueous phase since the onium salt and its derivatives are soluble in organic phase. The gross yield of isolated peptide is approximately 85%.

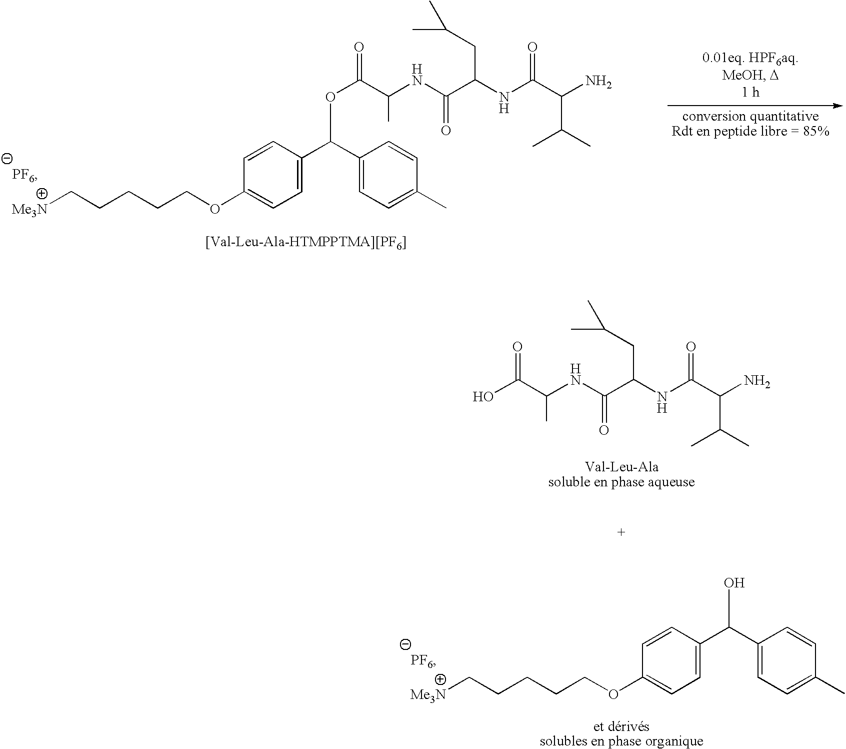

After cleavage, three onium salts are obtained: the alcohol [HTMPTTMA][PF₆] (approximately 35%), the methyl ether [Me-HTMPTTMA][PF₆] (approximately 60%) and the dimer [HTMPTTMA-O-HTMPTTMA][PF₆] (approximately 5%) identified by NMR and HPLC/MS. The addition of thionyl chloride to this mixture makes it possible to quantitatively obtain the chlorinated derivative, which is the precursor making it possible to recommence a new peptide synthesis. The regeneration of the support is therefore possible.

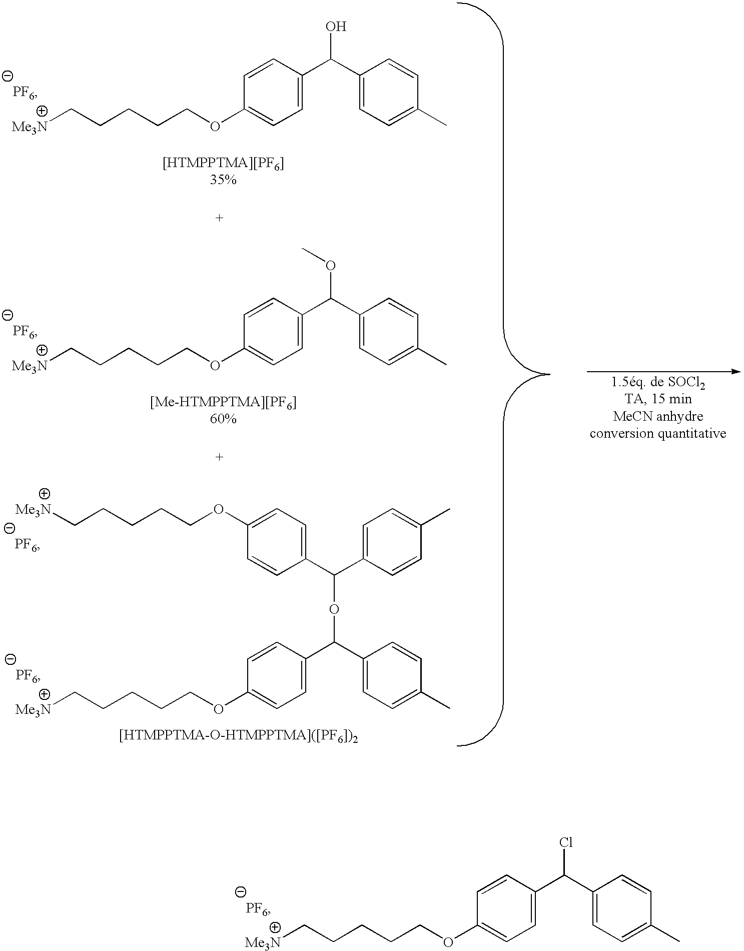

F-Study of the racemization:

In order to validate the methodology of peptide synthesis on onium salt support, it is essential to study the racemization, which is an important parameter in peptide synthesis.

Marfey has described a method which makes it possible not only to determine the racemization level during the grafting of the first amino acid onto the support, but also to study the racemization during the peptide synthesis. The principle is the following: The amino acid to be analyzed reacts with Marfey's reagent in the presence of a base in order to form the corresponding diastereoisomer which strongly absorbs UV at 340 nm (see diagram below). The latter is injected into reversed-phase HPLC. The retention time of the L-L diastereoisomer is less than that of the D-L: the intramolecular interactions by H bonds are stronger for this last diastereoisomer, which makes it more hydrophobic, it therefore interacts more strongly with the HPLC column and therefore its retention time is greater. This method has the advantage of being sensitive (the chromophore formed strongly absorbs UV, and only the Marfey's reagent which has not reacted is capable of interfering at this wavelength), effective (the Marfey's reagent is very reactive) and rapid.

This method was generally applied to the peptide racemization study.

The diagram below represents the grafting of the chromophore by reaction between the amino acid to be analyzed and Marfey's reagent:

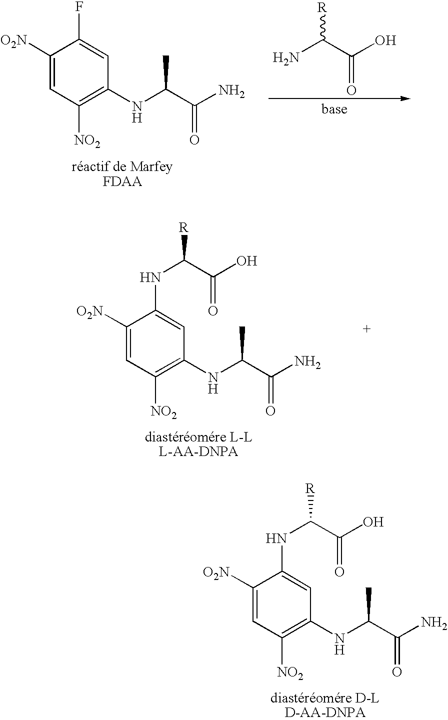

The study was carried out on the model peptide Val-Leu-Ala.

Firstly, a (commercial) racemic mixture of alanine was reacted with Marfey's reagent then injected into HPLC as a reference. The HPLC conditions were optimized for this mixture. After several measurements, the percentage of the areas of the peaks of L-Ala-DNPA and D-Ala-DNPA are respectively 48% and 51% (statistical values) compared with an expected 50% for each. The uncertainty is ±1.5%, which is relatively significant for a racemisation study, but which will all the same make it possible to come to a first serious estimation.

Then, the Fmoc-L-alanine was grafted to the support [HTMPPTMA][PF₆] under the conditions previously described, then the amine function was deprotected and the amino acid was cleaved from the salt with a dedicated task. The diastereoisomer was synthesized by reaction between the released alanine and the reagent FDAA according to the conditions described by Marfey, then it was injected into HPLC under the conditions C (see hereafter—experimental part). 1.3% D-Ala-DNPA is obtained, which is of the order of the margin of error of 1.5%: the racemization seems to be negligible during the grafting stage.

Peptide synthesis was continued starting from [L-Ala-HTMPPTMA][PF₆]. The nature of the coupling reagents influences the racemization, which is why the peptide couplings were carried out in parallel or with DIC/HOBt, or with HBTU. Thus, the dipeptide L-Leu-L-Ala was cleaved from the support, reacted with the Marfey's reagent and injected into HPLC under the conditions D (see hereafter—experimental part). The retention time of L-Leu-L-Ala-DNPA is much greater than that of L-Ala-DNPA, which is why it was necessary to adapt the elution conditions (eluent 15/85: acetonitrile/water for Ala-DNPA against 20/80: acetonitrile/water for Leu-Ala-DNPA). The peak of D-Leu-L-Ala-DNPA is not observed.

The same procedure was followed for the tripeptide L-Val-L-Leu-L-Ala. The tripeptides D-Val-L-Leu-L-Ala and L-Val-D-Leu-L-Ala were also synthesized on [HTMPPTMA][PF₆], grafted onto FDAA after cleavage from the support and injected into HPLC. The reference retention times are 19.1 min for D-Val-L-Leu-L-Ala-DNPA, which is not visible on the spectra of L-Val-L-Leu-L-Ala-DNPA, and 20.6 min for L-Val-D-Leu-L-Ala-DNPA, present at 1% on the spectra of L-Val-L-Leu-L-Ala-DNPA; the peptide couplings were carried out by HOBt/DIC or HBTU, which is of the order of magnitude of the margin of error.

These results make it possible to state that the racemization is weak at the grafting stage and during the peptide coupling reactions.

### IV-CONVERGENT SYNTHESIS

A-Convergent synthesis in the literature:

The synthesis in solution of peptides of less than five amino acids is carried out by a linear strategy since a convergent approach is preferable for the peptides the chain of which is longer. In this case, a judicious choice of the fragments (size, connections at the level of amino acids not very sensitive to racemization), the protective groups and the coupling methods is of prime importance. The main problems are the racemization and above all the low solubility of the fragments.

The solid-support synthesis by linear strategy is often poorly suited to the production of long peptides: the final peptide is contaminated by peptides the chain of which comprises deletions of amino acids, and the purification is often problematic.

This has led to the development of convergent peptide synthesis in solid phase (CPSSP) or synthesis in solid phase by fragment condensation (SPFC). This so-called hybrid approach combines synthesis in solution and synthesis on solid supports: the fragments are synthesized on solid support (in general they comprise less than 15 amino acids) then,
either a single one of the two fragments is cleaved; the other remains grafted to the resin and the following peptide coupling is carried out in heterogeneous phase;
or both are cleaved and coupled in solution (method sometimes advantageous when the yield of the peptide coupling in heterogeneous phase is weak (“difficult sequences”) and because the two fragments can be used in equimolar quantities).

The diagram represents the principle of convergent synthesis on solid phase:

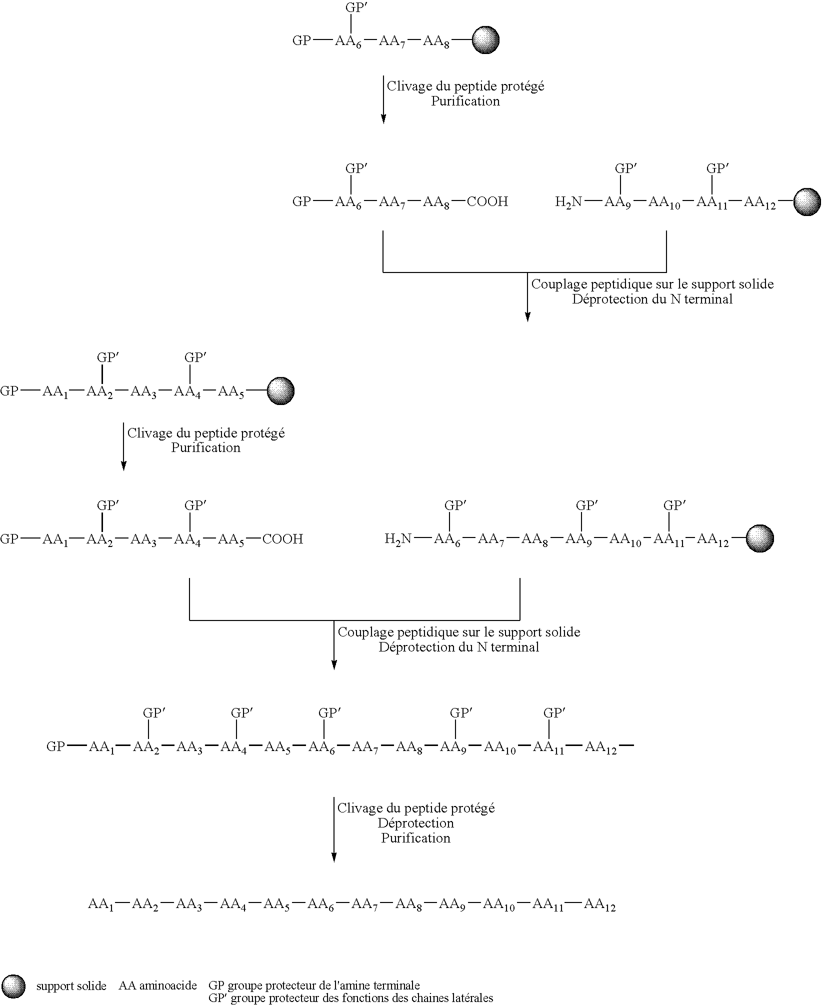

There are more and more solid supports which can be cleaved under mild conditions (SASRIN resin for example) which makes it possible to retain the protective groups of the functional chains of the fragments, which are essential for the remainder of the synthesis. Each fragment can be purified and characterized individually. The introduction of the first fragment can be carried out by synthesizing it by linear synthesis on the resin or by grafting it directly (the advantage is that the fragment was purified beforehand but in general the yields of the reactions of grafting fragments to a resin are low).

Various parameters must be studied scrupulously before the synthesis: the nature of the resin or resins, the fragments, the protective groups, the methods of peptide coupling and cleavage of the resins, the reaction time and the number of equivalents of fragments. It is essential that the free fragment is soluble in the solvent used for the peptide coupling in the following heterogeneous phase, and these problems of solubility at the origin of poor reactivities are the greatest limitation of the method, all the more so as they are not always foreseeable. The risks of racemization must also be taken into account.

The convergent synthesis can also involve reacting together two supported fragments. This is not possible starting from fragments bound to solid supports as these fragments are attached to distinct beads and the probability of their coming together is close to zero. However, the synthesis of biaryls by Suzuki coupling between an aryl iodide and a boronic acid each supported on a monomethoxypoly(ethylene glycol) was carried out in solution (K. D. Janda et al. Chem. Comm. 2003, 480-481) with yields varying from 72 to 95% with purities ranging from 50 to 95%. The purification by HPLC of the impure products has proved difficult. Another problem is linked to the very weak specific load of these supports due to their large molecular mass. The quantities of products involved are then homeopathic.

B-Convergent Synthesis Supported on Onium Salt:

The peptide synthesis on ammonium salt is carried out under homogeneous conditions. The convergent syntheses can therefore be carried out by coupling in solution supported peptides on onium salts having been synthesized, one by reverse route, the other by direct route. Two trisupported peptides were thus coupled, thus forming a hexapeptide. The reaction was carried out with 1.0 equivalent of each supported peptide; 1.5 equivalents of DCC, HOBt and TEA then left overnight at ambient temperature.

The mass spectrum of the crude reaction product shows the absence of the two initial peptides involved and that of the expected peptide (HRMS of (C₆₈H₁₀₈N₈O₁₁): [C⁺⁺] m_{theoretical}=1212.8138; m/z_{theoretical}=606.4069; m/zₑₓₚₑᵣᵢₘₑₙₜₐₗ=606.4063): the reaction is total. This shows the feasibility of the convergent synthesis with this novel technology and opens the route to the synthesis of longer peptides containing up to 30 amino acids.

The diagram which follows represents an example of hexapeptide originating from a convergent synthesis:

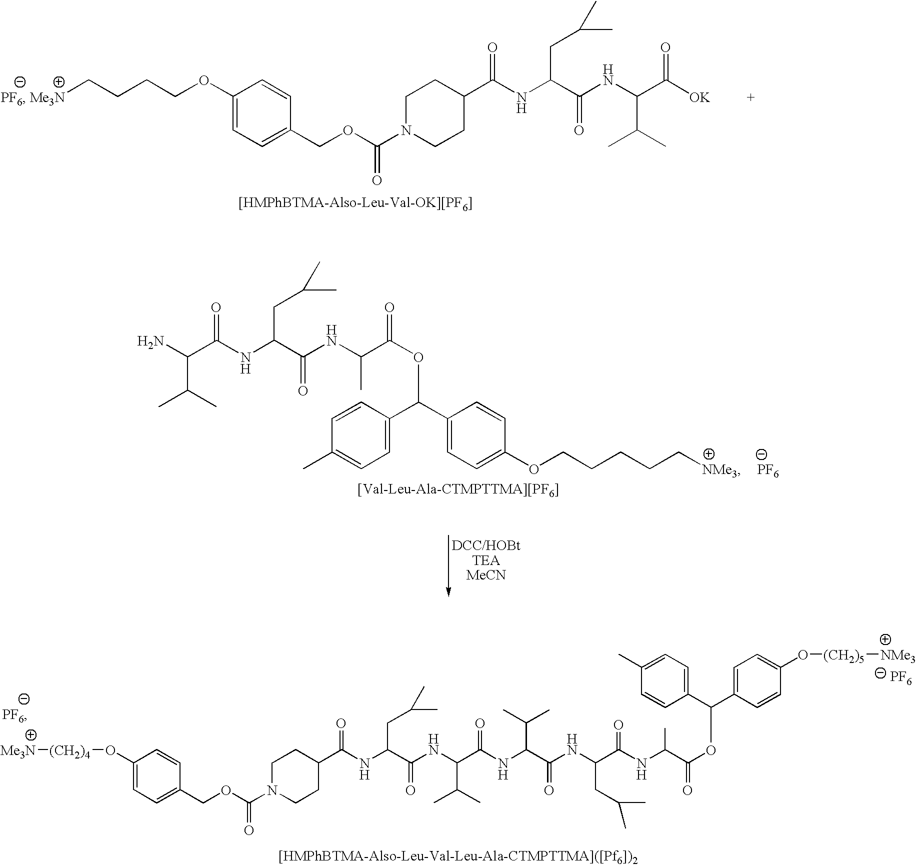

This work open the route to the convergent synthesis of peptides by coupling of supported fragments.

When the disupported peptide is obtained, the continuation of the convergent synthesis can be envisaged in selectively cleaving one of the two supports in order to obtain the monosupported peptide, which can then be coupled to another conveniently protected supported peptide, making it possible to extend the chain.

For example, the stability of the carbamate function serving for the grafting of the amino acid to the support [HMPhBTMA-Aiso-Leu-Val][PF₆] was tested under the conditions of cleavage of the ester function developed for the SOTS [Val-Leu-Ala-CTMPTTMA][PF₆] used for the direct route synthesis (0.01 eq. of HPF₆ in methanol at reflux). The carbamate is not cleaved under these conditions. It is therefore possible to selectively cleave the benzhydryl ester function of [HMPhBTMA-Aiso-Leu-Val-Val-Leu-Ala-CTMPTTMA]([PF₆])₂ and to continue the synthesis by the terminal acid function by carrying out a second coupling reaction with a third fragment.

### Experimental Part

1. Equipment

1.1. NMR spectrometers
Bruker ARX200 high field spectrometer (200.1 MHz for the proton; 50.0 MHz for carbon 13).
Bruker AC300P high field spectrometer with auto-sampler and BBO ATMA automatically tuneable multinuclear probe (300.1 MHz for the proton; 75.5 MHz for carbon 13; 282.4 MHz for fluorine 19 and 121.5 MHz for phosphorus 31).
Bruker AVANCE 500 high field spectrometer with 5 mm multinuclear TBI triple probe (500 MHz for the proton, 125 MHz for carbon 13).
The δ chemical shifts are expressed in parts per million (ppm):
   with respect to tetramethylsilane used as external reference for proton NMR and carbon 13 NMR.
   with respect to 85% phosphoric acid in water used as external reference for phosphorus 31 NMR.
   with respect to CFCl₃ as external reference for fluorine 19 NMR.
   with respect to ether trifluoroborate used as external reference for boron 11 NMR.
The coupling constants are expressed in Hertz (Hz). The following abbreviations were used to describe the multiplicity of the signals: s singlet, d doublet, t triplet, q quadruplet, m multiplet.

1.2. Mass Spectrometers
Electronic impact: EI

VARIAN MAT 311 double focussing high resolution mass spectrometer (with reversed NIER-JOHNSON BE geometry) belonging to the Centre Regional de Mesures

Physiques de l'Ouest. The beam energy is 70 eV, the strength of the emission current 300 μA and the ion acceleration voltage is 3,000 V.
LSIMS Source (Liquid Secondary Ion Mass Spectrometry)

MS/MS ZABSpec TOF Micromass high resolution mass spectrometer having EBE TOF geometry (magnetic and electric sectors with orthogonal time of flight) belonging to the Centre Régional de Mesures Physiques de l'Ouest.

The high and low mass spectra were produced with LSIMS ionization in positive mode using a cesium gun. m-nitrobenzyl alcohol was used as a matrix. The ions are accelerated with a voltage of 8,000 V. The determination of the precise masses is carried out by scanning the electric field using PEG ions as internal reference.
Electrospray Source: ESI

MS/MS ZABSpec TOF Micromass high resolution mass spectrometer having EBE TOF geometry (magnetic and electric sectors with orthogonal time of flight) belonging to the Centre Regional de Mesures Physiques de l'Ouest. The determination of the precise masses is carried out by scanning the electric field using polyethylene glycol ions as internal reference.

1.3. Elementary Analysis

Flash EA1112 CHNS/O microanalyzer belonging to the Centre Regional de Mesures Physiques de l'Ouest.

1.4. Chromatography

1.4.1. HPLC/MS

HPLC Waters 2695, column C18 3×50 mm Hypersil Gold 3 μm, flow rate of 900 μL/min, gradient A: H₂O (0.1% HCOOH)/B: MeCN: 5 to 90% of B in 5 minutes. Simultaneous UV and ELS detection. Ionization: positive and negative electrospray.

1.4.2. HPLC

Two types of column were used:
Waters Nova-Pak 4 μm C18 3.9×150 mm Column for:

isocratic HPLC: Waters 515 HPLC Pump, Milton Roy UV detector.

Conditions A: for peptides supported on [HMPhBTMA][NTf₂]: acetonitrile/water mixture 60/40: containing 1% acetic acid and 10 mmol.L⁻¹ of ammonium acetate. Flow rate of 1 mL/min. UV detection at 254 nm.

Conditions B: for peptides supported on [HTMPTTMA][PF₆]: acetonitrile/water mixture 70/30 containing 1.1% acetic acid and 20 mmol.L⁻¹ of ammonium acetate. Flow rate of 0.75 mL/min. UV detection at 230 nm.

Conditions C: for the amino acid racemization study: acetonitrile/water mixture 15/85 containing 1.1% acetic acid and 20 mmol.L⁻¹ of ammonium acetate. Flow rate of 1.5 mL/min. UV detection at 340 nm.

Conditions D: for the dipeptide racemization study: acetonitrile/water mixture 20/80 containing 1.1% acetic acid and 20 mmol.L⁻¹ of ammonium acetate. Flow rate of 1.5 mL/min. UV detection at 340 nm.

Conditions E: for the tripeptide racemization study: acetonitrile/water mixture 25/75 containing 1.1% acetic acid and 20 mmol.L⁻¹ of ammonium acetate. Flow rate of 1.5 mL/min. UV detection at 340 nm.
HPLC eluent gradient: Waters 2996, console: Waters 600 controller, injection: Waters Delta 600 for:

Conditions F: for peptides supported on [CTMPTTMA][PF₆]: gradient A: H₂O (1.1% acetic acid and 20 mmol.L⁻¹ of ammonium acetate)/B: MeCN: 40 to 100% B for 20 minutes then 10 minutes at 100% B. Flow rate of 1 mL/min.

Conditions G: for the free peptides: gradient A: H₂O (1% TFA)/B: MeCN (1% TFA): 0 to 100% B in 30 minutes. Flow rate of 1 mL/min.
Macherey-Nagel Nucleodur 100 Å 5 μm C18 50×4.6 mm phase column for: HPLC eluent gradient: Waters 515 HPLC Pump

Conditions H: for peptides supported on [CTMPTTMA][PF₆]: gradient A: H₂O (0.07% TFA)/B: MeCN (0.07% TFA): 0 to 100% B in 30 minutes. Flow rate of 1 mL/min.

1.4.3. Flash Chromatography

Activated neutral aluminium oxide column, 50 to 200 μm.

1.5. Melting Points

The melting points were measured using a Koffler bench.

1.6. Solvents

Anhydrous ether and THF are distilled under argon on sodium/benzophenone. Anhydrous DCM and isopropanol are distilled under argon on CaH₂.

2. Procedures

The following procedures are described in the case where the salt with a dedicated task is used alone as soluble support. The procedures are exactly identical when a matrix (ionic liquid, for example [tmba][NTf₂], or onium salt, for example [tmba][PF₆]) is added.

The concentrations of the SOTS solutions in the molecular solvents are 0.1 mol/L. The purity of the SOTS is greater than 95% according to the NMR spectra.

2.1. Synthesis of the Salts with a Dedicated Task
General procedure 1 for the quaternization reaction

1.0 eq. of halogenated derivative is introduced into a Schlenk tube. 2.0 eq. of a 45% aqueous solution of trimethylamine and acetonitrile are then added. The medium is taken to 70° C. for 18 hours. The solvents are then evaporated off under vacuum. Ether is added to the residue which crystallizes. The solid is filtered and washed with ether, before being placed in a desiccator overnight.
General procedure 2 for the metathesis reaction between a halide and a trifluoromethane sulphonate:

1.0 eq. of the onium halide is dissolved in a minimum amount of water. 1.1 eq. of LiNTf₂ are dissolved in a minimum amount of water then the two solutions are mixed. The medium is stirred for one hour at ambient temperature (AT). The expected salt is oily and settles at the bottom of the flask. Dichloromethane is added to the reaction medium.

If the salt is soluble in DCM, the aqueous and organic phases are separated. The organic phase is dried over sodium sulphate. The mixture is filtered. The dichloromethane is evaporated off.

If the salt is insoluble in DCM, the ionic liquid is phase separated from the aqueous phase and from the DCM phase, then acetonitrile and Na₂SO₄ are added to it. The solution is filtered then the acetonitrile is evaporated off.

2.1.2. Synthesis of [HPrTMA][NTf₂]

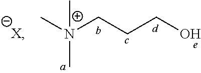

### X═Cl: [HPrTMA][Cl]

Procedure: cf general procedure 1 using 3-chloropropanol 4.

Yield is 82%.

White solid. MP=158-160° C.

NMR¹H (200 MHz, D₂O): δ(Hₐ)=3.00 (s, 9H); δ(H_{b})=3.30 (m, 2H); δ(W=1.92 (m, 2H); δ(H_{d})=3.60 (t, J=7.1, 2H).

NMR¹³C (50 MHz, D₂O): δ(Cₐ)=53.31 (t, J_{N-C}=4.1); δ(C_{b})=58.52; δ(C_{c})=25.68; δ(C_{d})=64.52.

HRMS (FAB): [2C⁺,A⁻] (C₁₂H₃₂N₂O₂C1) m/zₜₕ=271.2152; m/zₑₓₚ=271.2149.

### X═NTf₂: [HPrTMA][NTf₂]

Procedure: cf general procedure 2 using (3-hydroxy-propyl)-trimethylammonium chloride [HPrTMA][C1]. The yield is 90%.

colourless viscous oil

NMR¹H (200 MHz, acetone d⁶): δ(Hₐ)=3.25 (s, 9H); δ(H_{b+d})=3.50-3.80 (m, 2H+2H); δ(H_{c})=2.10 (m, 2H).

NMR¹³C (50 MHz, acetone d⁶): δ(Cₐ)=54.27 (t, J_{N-C}=4.1); δ(C_{b})=60.05; δ(C_{c})=29.14; δ(C_{d})=66.09; δ(C_{NTf2})=121.05 (q, J_{C-F}=321.2).

HRMS (FAB): [2C⁺,A⁻] (C₁₄H₃₂N₃O₆F₆S₂) m/Zₜₕ=516.1636; m/Zₑₓₚ=516.1632.

2.1.3. Synthesis of [HBuTMA][NTf₂]

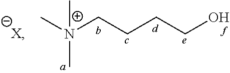

### X═Cl: [HBuTMA][Cl]

Procedure: cf general procedure 1 using 4-chlorobutanol 5. The yield is 94%.

hygroscopic white solid. MP=118-120° C.

NMR¹H (200 MHz, D₂O): δ(Hₐ)=3.25 (s, 9H); δ(H_{b})=3.45 (m, 2H); δ(W=1.69 (m, 2H); δ(H_{d})=1.95 (m, 2H); δ(H_{c})=3.60 (t, J=7.2, 2H).

NMR¹³C (50 MHz, D₂O): δ(Cₐ)=53.18 (t, J=4.1); δ(C_{b})=61.11; δ(C_{c})=19.50; δ(C_{d})=28.43; δ(C_{c})=66.66.

### X═NTf₂: [HBuTMA][NTf₂]

Procedure: cf general procedure 2 using (4-hydroxy-butyl)-trimethylammonium chloride [HBuTMA][C1]. The yield is quantitative.

colourless viscous oil

NMR¹H (200 MHz, acetone d⁶): δ(Hₐ)=3.38 (s, 9H); δ(H_{b+e+f})=3.57-3.69 (m, 2H+2H+1H); δ(H_{c})=1.63 (m, 2H); δ(H_{d})=2.04 (t, J=6.1, 2H).

NMR¹³C (50 MHz, acetone d⁶): δ(Cₐ)=52.99 (t, J=3.9); δ(C_{b})=61.14; δ(C_{c})=19.82; δ(C_{d})=29.30; δ(C_{c})=66.80; δ(C_{NTf2})=120.36 (q, J_{C-F}=320.9).

NMR¹⁹F (282 MHz, acetone d⁶): δ(F_{NTf2})=−79.91.

HRMS (FAB): [2C⁺,A⁻] (C₁₆H₃₆N₃O₆F₆S₂) m/zₜₕ=544.1950; m/zₑₓₚ=544.1928.

2.1.4. Synthesis of [HHeTMA][NTf₂]

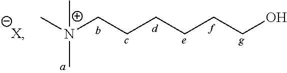

### X═Cl: [HHeTMA][Cl]

Procedure: cf general procedure 1 using 6-chlorohexanol 6. The yield is quantitative.

Hygroscopic white solid. MP=178-180° C.

NMR¹H (200 MHz, D₂O): δ(Hₐ)=3.02 (s, 9H); δ(H_{b})=3.23 (m, 2H); δ(H_{c})=1.48 (m, 2H); δ(H_{d+e})=1.27-1.34 (m, 2H+2H); δ(H_{f})=1.72 (m, 2H); δ(H_{g})=3.51 (t, J=6.2, 2H).

NMR¹³C (75 MHz, D₂O): δ(Cₐ)=52.85 (t, J=3.6); δ(C_{b})=66.00; δ(C_{c})=22.26;

δ(C_{d})=25.26; δ(Cₑ)=24.61; δ(C_{f})=31.07; δ(C_{g})=61.56.

HRMS (FAB): [M⁺] (C₁₈H₄₄N₂O₂C1) m/z_{tb}=355.3091; m/zₑₓₚ=355.3093.

### X═NTf₂: [HHeTMA][NTf₂]

Procedure: cf general procedure 2 using (6-hydroxy-hexyl)-trimethylammonium chloride [HHeTMA][Cl]. The yield is 95%.

colourless viscous oil

NMR¹H (200 MHz, acetone d⁶): δ(Hₐ)=3.36 (s, 9H); δ(H_{b+g+h})=3.52-3.61 (m, 2H+2H+1H); δ(H_{c+d+e})=1.36-1.68 (m, 2H+2H+2H); δ(H_{f})=2.00 (m, 2H).

NMR¹³C (75 MHz, acetone d⁶): δ(Cₐ)=52.55; δ(C_{b})=66.51; δ(C_{c})=22.53; δ(C_{d})=25.65; δ(Cₑ)=25.02; δ(C_{f})=32.20; δ(C_{g})=61.45; δ(C_{NTf2})=119.95 (q, J_{C-F}=321.0).

NMR¹⁹F (282 MHz, acetone d⁶): δ(F_{NTf2})=−79.90.

2.1.5. Synthesis of [HPeTMA][NTf₂]

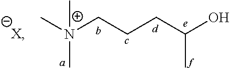

### X═Cl: [HPeTMA][Cl]

Procedure: cf general procedure 1 using 5-chloropentan-2-ol 8. The yield is 95%.

hygroscopic white solid

NMR¹H (200 MHz, D₂O): δ(Hₐ)=3.05 (s, 9H); δ(H_{b})=3.27 (m, 2H); δ(H_{c})=1.46 (m, 2H); δ(H_{d})=1.78 (m, 2H); δ(Hₑ)=3.81 (q, J=6.3, 1H); δ(H_{f})=1.13 (d, J=6.2, 3H).

NMR¹³C (75 MHz, D₂O): δ(Cₐ)=52.87 (t, J_{N-C}=3.8); δ(C_{b})=66.46; δ(C_{c})=18.91; δ(C_{d})=34.26; δ(Cₑ)=66.98; δ(C_{f})=21.95.

HRMS (LSIMS) of (C₈H₂₀NO): [M⁺] m/z_{theoretical}=146.1545; m/zₑₓₚₑᵣᵢₘₑₙₜₐₗ=146.1547.

### X═NTf₂: [HPeTMA][NTf₂]

Procedure: cf general procedure 2 using (4-hydroxy-pentyl)-trimethylammonium chloride [HPeTMA][Cl]. The yield is 95%.

viscous colourless oil

NMR¹H (200 MHz, acetone d⁶): δ(Hₐ)=3.39 (s, 9H); δ(H_{b})=3.60 (m, 2H); δ(H_{c})=1.52 (m, 2H); δ(H_{d})=2.04 (m, 2H); δ(Hₑ)=3.84 (m, 1H); δ(H_{f})=1.18 (d, J=6.1, 3H); δ(H_{g})=3.70 (d, J=4.7, 1H).

NMR¹³C (75 MHz, acetone d⁶): δ(Cₐ)=52.28 (t, J_{N-C}=3.8); δ(C_{b})=66.28; δ(C_{c})=18.90; δ(C_{d})=34.68; δ(Cₑ)=65.96; δ(C_{f})=22.50; δ(C_{NTf2})=119.60 (q, J_{C-P=}320.7).

HRMS (ESI) of (C₁₈H₄₀N₃O₆F₆S₂): [2C⁺,A⁻] m/Z_{theoretical}=572.2263; m/zₑₓₚₑᵣᵢₘₑₙₜₐₗ 572.2266.

2.1.6. Synthesis of [HMPeTMA][X]

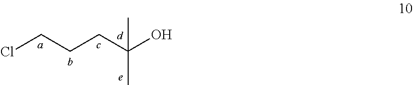

Procedure: 30.6 mL (492 mmol; 3.0 eq.) of methyl iodide in anhydrous ether are added dropwise to 12.75 g (252 mmol; 3.2 eq.) of magnesium activated beforehand by heating under vacuum. The solution is then stirred for 30 minutes at AT. A solution of 20 mL (164 mmol; 1.0 eq.) of methyl 4-chlorobutyrate 9 in 250 mL of anhydrous THF is added dropwise at AT then the medium is taken to reflux overnight. The mixture is neutralized with methanol at 0° C. then the solvents are evaporated off under vacuum. Ether is added to the residue and the mixture is filtered on frit. The solvents are evaporated off under vacuum. 21.6 g (95%) of oil are obtained.

yellow oil

NMR¹H (200 MHz, CDCl₃): δ(Hₐ)=3.58 (t, J=6.6, 2H); δ(H_{b})=1.59 (m, 2H);

δ(H_{c})=1.89 (m, 2H); δ(Hₑ)=1.23 (s, 6H).

NMR¹³C (50 MHz, CDCl₃): δ(Cₐ)=45.69; δ(C_{b})=27.69; δ(C_{c})=40.89; δ(C_{d})=70.42; δ(Cₑ)=29.20.

HRMS (ESI) of (C₅H₁₀OCl): [M-.CH₃⁺] m/z_{theoretical}=121.0420; m/zₑₓₚₑᵣᵢₘₑₙₜₐₗ=121.0412.

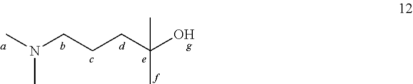

Procedure: 2.0 g (14.6 mmol; 1.0 eq.) of 5-chloro-2-methylpentan-2-ol 10 are dissolved in 20 mL of acetonitrile then 5.1 g (36.6 mmol; 2.5 eq.) of K₂CO₃ and 4.6 mL (36.6 mmol; 2.5 eq.) of a 40% aqueous solution of dimethylamine 11 are added. The medium is taken to 50° C. overnight then it is filtered. The filtrate is extracted with acetonitrile and the filtrate is dried over sodium sulphate. The mixture is filtered and the solvents are evaporated off under vacuum. 1.8 g (85%) of 5-dimethylamino-2-methylpentan-2-ol 12 is obtained.

yellow oil

NMR¹H (200 MHz, CDCl₃): δ(Hₐ)=2.25 (s, 6H); δ(H_{b})=2.33 (m, 2H); δ(H_{c+d})=1.61-1.63 (m, 2H+2H); δ(H_{f})=1.21 (s, 6H); δ(H_{g})=5.67 (m, 1H).

NMR¹³C (50 MHz, CDCl₃): δ(Cₐ)=45.26; δ(C_{b})=43.31; δ(C_{c})=22.65; δ(C_{d})=60.63; δ(Cₑ)=68.81; δ(C_{f})=29.99.

HRMS (1E) of (C₈H₁₉NO): [M⁺]m/z_{theoretical}=145.1467; m/zₑₓₚₑᵣᵢₘₑₙₜₐₗ=145.1469.

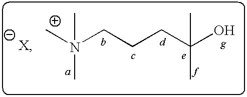

### X═Cl: [HMPeTMA][Cl]

Procedure: cf general procedure 1 using 5-chloro-2-methylpentan-2-ol 10. The yield is 70%.

hygroscopic off-white solid. MP=132-134° C.

NMR¹H (200 MHz, D₂O): δ(Hₐ)=3.04 (s, 9H); δ(H_{b})=3.25 (m, 2H); δ(H_{c})=1.43 (m, 2H); δ(H_{d})=1.77 (m, 2H); δ(H_{f})=1.16 (s, 6H).

NMR¹³C (75 MHz, D₂O): δ(Cₐ)=52.89 (t, J_{N-C}=_{T-C}=3.9); δ(C_{b})=66.76; δ(C_{c})=17.86; δ(C_{d})=38.72; δ(Cₑ)=70.98; δ(C_{f})=27.73.

LRMS (LSIMS) of (C₁₈H₄₄N₂O₂C1): [2C⁺, Cl⁻] m/z_{theoretical}=355; m/zₑₓₚₑᵣᵢₘₑₙₜₐₗ=355.3.

### X═NTf₂: [HMPeTMA][NTf₂]

Procedure: cf general procedure 2 using 4-hydroxy-4-methyl-pentyl-trimethylammonium chloride. The yield is 92%.

pale yellow viscous oil.

NMR¹H (200 MHz, acetone d₆): δ(Hₐ)=3.39 (s, 9H); δ(H_{b})=3.58 (m, 2H); δ(H_{c})=2.06 (m, 2H); δ(H_{d})=1.53 (t, J=7.8, 2H); δ(H_{f})=1.23 (s, 6H), δ(H_{g})=3.45 (s, 1H).

NMR¹³C (50 MHz, D₂O): δ(Cₐ)=53.13 (t, J_{N-C}=4.0); δ(C_{b})=67.03; δ(C_{c})=18.14; δ(C_{d})=38.98; δ(Cₑ)=71.30; δ(C_{f})=27.89; δ(C_{NTf2})=119.59 (q, J=319.6).

NMR¹⁹F(282 MHz, acetone d₆): δ(CF₃)=−79.90

HRMS (LSIMS) of (C₂₀H₄₄N₃O₆F₆S₂): [2C⁺, NTf₂⁻] m/z_{theoretical}=600.2576; m/zₑₓₚₑᵣᵢₘₑₙₜₐₗ=600.2583.

### X═BF₄: [HMPeTMA][BE₄]

Procedure: 3.0 g (15.3 mmol, 1.0 eq.) of 4-hydroxy-4-methyl-pentyl-trimethylammonium chloride is dissolved in water then 2.5 mL (19.9 mmol, 1.3 eq.) of a 50% aqueous solution of HBF₆ are added dropwise to the solution which is stirred for three hours at AT. The solvents are evaporated off under vacuum. The residue is washed three times with ether then placed in a desiccator. 3.0 g (80%) of product is obtained.

hygroscopic white solid. MP=92-94° C.

NMR¹H (200 MHz, acetone d₆): δ(Hₐ)=3.36 (s, 9H); δ(H_{b})=3.56 (m, 2H); δ(H_{c})=1.53 (m, 2H); δ(H_{d})=2.00 (m, 2H); δ(H_{f})=1.23 (s, 6H); δ(H_{g})=3.18 (s, 1H).

NMR¹³C (50 MHz, acetone d₆): δ(Cₐ)=53.80 (t, J_{N-C}=4.0); δ(C_{b})=68.18; δ(C_{c})=19.28; δ(C_{d})=40.83; δ(Cₑ)=70.23; δ(C_{f})=30.08.

NMR¹¹B (96 MHz, acetone d⁶): δ(B)=−0.97.

HRMS (LSIMS) of (C₁₈H₄₄N₂O₂F₄B): [2C⁺, BF₄⁻] m/z_{theoretical}=407.3432; m/zₑₓₚₑᵣᵢₘₑₙₜₐₗ=407.3441.

### X═PF₆: [HMPeTMA][PF₆]

Procedure: 200 mg (1.0 mmol, 1.0 eq.) of 4-hydroxy-4-methyl-pentyl-trimethylammonium chloride is dissolved in water then 0.2 mL (1.3 mmol, 1.3 eq.) of aqueous solution of HPF₆ to 60% is added dropwise to the solution which is stirred for five hours at AT. The solvents are evaporated off under vacuum. The residue is washed three times with ether then dried with a desiccator. 0.25 g (80%) of product is obtained.

hygroscopic off-white solid. MP=191-193° C.

NMR¹H (200 MHz, D₂O): δ(Hₐ)=3.05 (s, 9H); δ(H_{b})=3.25 (m, 2H); δ(H_{c})=1.44 (m, 2H); δ(H_{d})=1.78 (m, 2H); δ(H_{f})=1.17 (s, 6H).

NMR¹³C (50 MHz, D₂O): δ(Cₐ)=53.10 (t, J_{N-C}=4.0); δ(C_{b})=67.05; δ(C_{c})=18.11; δ(C_{d})=38.97; δ(Cₑ)=71.30; δ(C_{f})=27.86.

NMR³¹P (121 MHz, D₂O): δ(P)=−144.97 (seven, J=708).

LRMS (LSIMS) of (C₉H₂₂Nₒ): [C⁺] m/z_{theoretical}=160; m/zₑₓₚₑᵣᵢₘₑₙₜₐₗ=160.

### X═I: [HMPeTMA][I]

Procedure: 8.0 g (55.2 mmol, 1.0 eq.) of 5-dimethylamino-2-methylpentan-2-ol 12 is dissolved in 60 mL of acetonitrile then 4.1 mL (66.2 mmol, 1.2 eq.) of iodomethane is added to 0° C. A suspension appears. The medium returns to AT overnight then it is filtered on frit. The filtrate is washed with ether. 12.6 g (80%) of [HMPeTMA][I] is obtained.

hygroscopic white solid. MP=168-170° C.

NMR¹H (200 MHz, D₂O): δ(Hₐ)=3.18 (s, 9H); δ(H_{b})=3.39 (m, 2H); δ(H_{c})=1.56 (m, 2H); δ(H_{d})=1.89 (m, 2H); δ(H_{f})=1.29 (s, 6H).

NMR¹³C (50 MHz, D₂O): δ(Cₐ)=53.37 (t, J_{N-C}=4.0); δ(C_{b})=67.12; δ(C_{c})=18.28; δ(C_{d})=39.06; δ(Cₑ)=71.43; δ(C_{f})=28.10.

Elementary analysis: Theoretical C, 37.64%—H, 7.72%—N, 4.88%.
Measured C, 37.51%—H, 7.79%—N, 4.98%.

### X=MeSO₄: [HMPeTMA][MeSO₄]

Procedure: 3.8 g (26.2 mmol; 1.0 eq.) of 5-dimethylamino-2-methylpentan-2-ol 12 is dissolved in 30 mL of anhydrous ether then 2.7 mL (28.8 mmol; 1.1 eq.) of freshly distilled dimethyl sulphate are added at 0° C. A suspension appears. The medium is stirred for 45 minutes at AT then it is filtered on frit. The filtrate is washed with ether. 5.9 g (83%) of [HMPeTMA][MeSO₄] is obtained.

hygroscopic white solid. MP=82-84° C.

NMR¹H (200 MHz, D₂O): δ(Hₐ)=3.03 (s, 9H); δ(H_{b})=3.23 (m, 2H); δ(H_{c})=1.41 (m, 2H); δ(H_{d})=1.76 (m, 2H); δ(H_{f})=1.15 (s, 6H); δ(H_{MeSO4})=3.75 (s, 3H).

NMR¹³C (50 MHz, D₂O): δ(Cₐ)=53.13 (t, J_{N-C}=4.0); δ(C_{b})=67.07; δ(C_{c})=18.15; δ(C_{d})=39.00; δ(Cₑ)=71.31; δ(C_{f})=27.93; δ(C_{MeSO4})=55.74.

HRMS (LSIMS) of (C₁₉H₄₇N₂O₆S): [2C⁺, MeSO₄⁻]m/z_{theoretical}=431.3155; m/zₑₓₚₑᵣᵢₘₑₙₜₐₗ=431.3148.

2.1.7. Synthesis of [HMPhBTMA][NTf₂] or [PF₆]

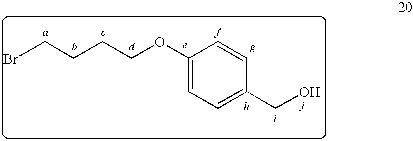

Procedure:

10.0 g (80.6 mmol) of 4-hydroxybenzylic alcohol 19 is dissolved in 125 mL of acetone. 19.2 mL (2.0 eq.; 161.1 mmol) of 1-4-dibromobutane 18 and 11.1 g (1.0 eq.; 80.6 mmol) of K₂CO₃ are added to the medium, which is then taken to reflux for 18 hours under stirring. The mixture is filtered. The disubstitution product precipitates from the filtrate and is eliminated by a second filtration. The acetone of the filtrate is then evaporated off. Pentane is added to the residue. The expected product 20 precipitates, is filtered, cleaned with pentane and dried overnight in a desiccator. 16.9 g (80%) of product is obtained.

off-white solid. MP<50° C.

NMR¹H (200 MHz, CDCl₃): δ(Hₐ)=3.50 (t, J=6.5, 2H); δ(H_{h+c})=1.91-2.21 (m, 2H+2H); δ(H_{d})=4.04 (t, J=5.7, 2H); δ(H_{f})=6.92 (d, J=8.6, 2H); δ(H_{g})=7.33 (d, J=8.9, 2H); δ(H₁)=4.66 (s, 2H); δ(Hⱼ)=1.65 (s, 1H).

NMR¹³C (75 MHz, CDCl₃): δ(Cₐ)=33.50; δ(C_{b})=27.89; δ(C_{c})=29.47; δ(C_{d})=66.89; δ(Cₑ)=158.49; δ(C_{f})=114.52; δ(C_{g})=128.68; δ(Cₕ)=133.20; δ(Cᵢ)=65.03.

HRMS (ESI) of (C₁₁H₁₃O₂Br): [M⁺.] m/z_{theoretical}=258.0255; m/zₑₓₚₑᵣᵢₘₑₙₜₐₗ=258.0266.

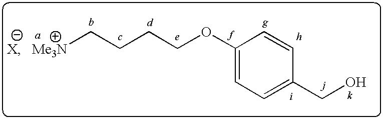

### X═Br: [HMPhBTMA][Br]

Procedure: cf general procedure 1 using [4-(4-bromobutoxy)phenyl]-methanol 12. The yield is 98%.

Hygroscopic white solid. MP=144-146° C.

NMR¹H (200 MHz, D₂O): δ(Hₐ)=3.04 (s, 9H); δ(H_{b})=3.32 (m, 2H); δ(H_{c+d})=1.74-1.99 (m, 2H+2H); δ(Hₑ)=4.06 (t, J=5.1, 2H); δ(H_{g})=6.95 (d, J=8.6, 2H); δ(Hₕ)=7.29 (d, J=8.6, 2H); δ(Hⱼ)=4.50 (s, 2H).

NMR¹³C (75 MHz, D₂O): δ(Cₐ)=52.82 (J_{C-N}=3.8); δ(C_{b})=66.14 (J_{C-N}=3.0); δ(C_{c})=19.34; δ(C_{d})=25.17; δ(Cₑ)=67.38; δ(C_{f})=157.59; δ(C_{g})=114.90; δ(Cₕ)=129.34; δ(Cᵢ)=133.09; δ(Cⱼ)=63.38.

HRMS (ESI) of (C₁₄H₂₄NO₂): m/z_{theoretical}=238.1807; m/zₑₓₚₑᵣᵢₘₑₙₜₐₗ=238.1811.

### X═PF₆: [HMPhBTMA][PF₆]

Procedure: 2.0 g (6.3 mmol) of 4-[4-(hydroxymethyl)phenoxy]-N,N,N-trimethylbutan-1-ammonium bromide [HMPhBTMA][Br] are dissolved in a minimum amount of water. 2.3 g (2.0 eq.; 12.6 mmol) of KPF₆ are then added. The reaction medium is stirred for two hours to AT. The 4-[4-(hydroxymethyl)phenoxy]-N,N,N-trimethylbutan-1-ammonium hexafluorophosphate formed precipitates, is filtered and washed three times with water and three times with ether before being place overnight in a desiccator. 3.11 g (85%) of solid are obtained.

hygroscopic white solid. MP=56-58° C.

NMR¹H (200 MHz, acetone d₆): δ(Hₐ)=3.41 (s, 9H); δ(H_{b})=3.71 (m, 2H); δ(H_{c})=1.93 (m, 2H); δ(H_{d})=2.19 (m, 2H); δ(Hₑ₊ₖ)=4.06-4.14 (m, 2H+1H); δ(H_{g})=6.92 (d, J=8.6, 2H); δ(Hₕ)=7.30 (d, J=8.4, 2H); δ(Hⱼ)=4.58 (d, J=5.5, 2H).

NMR¹³C (75 MHz, acetone d₆): δ(Cₐ)=52.66 (J_{C-N}=4.2); δ(C_{b})=66.26 (J_{C-N}=2.9); δ(C_{c})=19.73; δ(C_{d})=25.87; δ(Cₑ)=66.79; δ(C_{f})=158.03; δ(C_{g})=114.23; δ(Cₕ)=128.12; δ(Cᵢ)=134.67; 6 (Cⱼ)=63.48.

NMR³¹P (121 MHz, acetone d₆): δ(P_{PF6})=−144.24 (seven, J=708).

NMR¹⁹F (282 MHz, acetone d₆): δ(F_{PF6})=−72.457 (d, J=707).

HRMS (ESI) of (C₂₈H₄₈N₂O₄F₆P): [2C⁺,⁻PF₆] m/z_{theoretical}=621.3256; m/zₑₓₚₑᵣᵢₘₑₙₜₐₗ=631.3259.

### X═NTf₂: [HMPhBTMA][NTf₂]

Procedure: cf general procedure 2 using bromide of 4-[4-(hydroxymethyl)phenoxy]-N,N,N-trimethylbutan-1-ammonium [HMPhBTMA][Br].

[HMPhBTMA][NTf₂] is soluble in DCM. The yield is 95%.

slightly yellow viscous oil.

NMR¹H (300 MHz, acetone d₆): δ(Hₐ)=3.40 (s, 9H); δ(H_{b})=3.70 (m, 2H); δ(H_{c})=1.91 (m, 2H); δ(H_{d})=2.17 (m, 2H); δ(Hₑ₊ₖ)=4.03-4.11 (m, 2H+1H); δ(H_{g})=6.89 (d, J=8.6, 2H); δ(Hₕ)=7.28 (d, J=8.5, 2H); δ(Hⱼ)=4.56 (d, J=5.5, 2H).

NMR¹³C (75 MHz, acetone d₆): δ(Cₐ)=52.60; δ(C_{b})=66.22; δ(C_{c})=19.63; δ(C_{d})=25.69; δ(Cₑ)=66.81; δ(C_{f})=158.20; δ(C_{g})=114.41; δ(Cₕ)=128.61; δ(Cᵢ)=134.14; δ(Cⱼ)=63.57, δ(C_{NTf2})=120.02 (q, J=321.3).

NMR¹⁹F (282 MHz, acetone d₆): δ(F_{NTf2})=−79.88.

HRMS (ESI) of (C₃₀H₄₈N₃O₈F₆S₂): [2C⁺,NTf₂⁻] m/z_{theoretical}=756.2787; m/zₑₓₚₑᵣᵢₘₑₙₜₐₗ 756.2785.

2.1.8. Synthesis of [HTMPTTMA][Br] or [NTf₂]

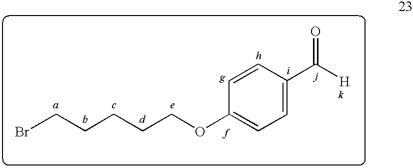

Procedure: 8.9 g (73 mmol) of p-hydroxybenzaldehyde 22 is dissolved in 115 mL of technical grade acetone. 20 mL (2.0 eq., 146 mmol) of 1-5-dibromopentane 21 and 10 g (1.0 eq., 73 mmol) of K₂CO₃ are added to the mixture which is taken to reflux for 18 hours with vigorous stirring. The solution, red at the start, turns yellow. The reaction medium is filtered. The expected product 23 (Tbp≈125° C. for P 0.05 mm Hg) is isolated after distillation of the residual oil with a Kügelrohr apparatus. 9.7 g (50%) of 4-(5-bromopentyloxy)-benzaldehyde 23 is obtained.

yellow oil

NMR¹H (200 MHz, CDCl₃): δ(Hₐ)=3.47 (t, J=6.6, 2H); δ(H_{b+d})=1.78-2.06 (m, 2H+2H); δ(H_{c})=1.69 (m, 2H); δ(Hₑ)=4.08 (t, J=6.3, 2H); δ(H_{g})=7.01 (d, J=8.8, 2H); δ(Hₕ)=7.86 (d, J=8.7, 2H); δ(Hₖ)=9.90 (s, 1H).

NMR¹³C (75 MHz, CDCl₃): δ(Cₐ)=33.67; δ(C_{b})=33.30; δ(C_{c})=24.61; δ(C_{d})=28.10; δ(Cₑ)=67.90; δ(C_{f})=163.91; δ(C_{g})=114.66; δ(Cₕ)=131.80; δ(Cᵢ)=129.71; δ(Cⱼ)=190.47.

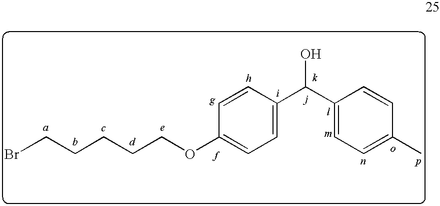

Procedure: A solution of 9.4 g (2.0 eq.; 12.7 mmol) of 4-bromotoluene in anhydrous ether is added dropwise to 1.4 g (2.1 eq.; 57.8 mmol) of magnesium deoxidized beforehand. The mixture is stirred for 30 minutes, then 7.5 g (1.0 eq.; 27.5 mmol) of 4-(5-bromopentyloxy)-benzaldehyde 23 dissolved in anhydrous ether is added dropwise at 0° C. The reaction medium is then stirred for one hour at AT, then the magnesium compound is hydrolyzed by the addition of methanol. The solvents of the medium are evaporated off under vacuum. Ether is added to the residue and this mixture is filtered on frit. After evaporation, 10.0 g (99%) of [4-(5-bromopentyloxy)-phenyl]-p-tolylmethanol 25 is obtained.

yellow oil

NMR¹H (200 MHz, CDCl₃): δ(Hₐ)=3.48 (t, J=6.7, 2H); δ(H_{b+d})=1.76-2.08 (m, 2H+2H); δ(H_{c})=1.65 (m, 2H); δ(Hₑ)=4.00 (t, J=6.3, 2H); δ(H_{g})=6.91 (d, J=9.5, 2H); δ(Hₕ₊ₘ₊ₙ)=7.17-7.34 (m, 2H+2H+2H); δ(Hⱼ)=5.82 (d, J=3.3, 1H); δ(Hₖ)=2.25 (d, J=3.5, 1H); δ(Hₚ)=2.38 (s, 3H).

NMR¹³C (50 MHz, CDCl₃): δ(Cₐ)=34.40; δ(C_{b})=33.06; δ(C_{c})=25.41; δ(C_{d})=29.01; δ(Cₑ)=68.13; δ(C_{f})=158.79; δ(C_{g})=114.88; δ(Cₕ)=128.43; δ(Cᵢ)=137.04; δ(Cⱼ)=75.98; δ(Cₗ)=141.95; δ(Cₘ)=127.03; δ(Cₙ)=129.63; δ(Cₒ)=137.38; δ(Cₚ)=21.78.

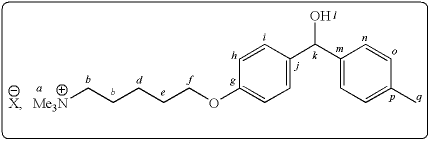

### X═Br: [HTMPPTMA][Br]

Procedure: 10.0 g (27.5 mmol) of [4-(5-bromopentyloxy)-phenyl]-p-tolylmethanol 25 are introduced into a Schlenk tube. 8.4 mL (2.0 eq.; 55.1 mmol) of a 45% aqueous solution of trimethylamine and 20 mL of acetonitrile are then added. The medium is taken to 70° C. for 18 hours. The solvents are then evaporated off under vacuum. The oily residue is washed with ether. 9.6 g (83%) of product are obtained.

very viscous yellow oil.

NMR¹H (200 MHz, acetone d₆): δ(Hₐ)=3.42 (s, 9H); δ(H_{b})=3.72 (m, 2H); δ(H_{c+e})=1.69-1.94 (m, 2H+2H); δ(H_{d})=1.51 (m, 2H); δ(H_{f})=4.01 (t, J=6.2, 2H); δ(Hₕ)=6.89 (d, J=8.8, 2H); δ(Hᵢ₊ₙ₊ₒ)=7.08-7.38 (m, 2H+2H+2H); δ(Hₖ)=5.73 (s, 1H); δ(H_{l+q})=2.03-2.34 (m, 3H+1H).

NMR¹³C (75 MHz, CD₃CN): δ(Cₐ)=52.74; δ(C_{b})=66.04; δ(C_{c})=22.38; δ(C_{d})=22.59; δ(Cₑ)=28.46; δ(C_{f})=67.54; δ(C_{g})=157.94; δ(Cₕ)=114.25; δ(Cᵢ)=126.56; δ(Cⱼ)=136.30; δ(Cₖ)=74.05; δ(Cₘ)=143.04; δ(Cₙ)=127.79; δ(Cₒ)=128.82; δ(Cₚ)=138.06; δ(C_{q})=20.38.

HRMS (ESI) of (C₂₂H₃₂NO₂): [C⁺] m/z_{theoretical}=342.2433; m/zₑₓₚₑᵣᵢₘₑₙₜₐₗ=342.2435.

X═NTf₂: [HTMPPTMA][NTf₂]

Procedure: cf general procedure 2 using {5-[4-(hydroxy-p-tolyl-methyl)-phenoxy]-pentyl}-trimethyl-ammonium bromide [HTMPPTMA][Br]. [HTMPPTMA][NTf₂] is soluble in DCM. The yield is 90%.

viscous yellow oil.

NMR¹H (300 MHz, acetone d₆): δ(Hₐ)=3.38 (s, 9H); δ(H_{b})=3.62 (m, 2H); δ(H_{c})=1.87 (m, 2H); δ(H_{d})=1.61 (m, 2H); δ(Hₑ)=2.08 (m, 2H); δ(H_{f})=4.01 (t, J=6.2, 2H); δ(Hₕ)=6.84 (d, J=8.7, 2H); δ(Hᵢ₊ₙ₊ₒ)=7.07-7.33 (m, 2H+2H+2H); δ(Hₖ)=5.74 (s, 1H); δ(H₁)=2.83 (s, 1H); δ(H_{q})=2.29 (s, 3H).

NMR¹³C (75 MHz, acetone d₆): δ(Cₐ)=52.71 (q, J=3.8); δ(C_{b})=66.50; δ(C_{c})=22.66; δ(C_{d})=22.39; δ(Cₑ)=28.49; δ(C_{f})=67.23; δ(C_{g})=158.16; δ(Cₕ)=114.12; δ(Cᵢ)=126.39; δ(Cⱼ)=136.24; δ(Cₖ)=74.80; δ(Cₘ)=142.83; δ(Cₙ)=127.70; δ(Cₒ)=128.76; δ(Cₚ)=137.81; δ(C_{q})=20.30; δ(C_{NTf2})=120.13 (q, J=321.3).

NMR¹⁹F (282 MHz, acetone d₆): δ(F_{NTf2})=−79.88.

LRMS (ESI) of (C₂₂H₃₂NO₂): [2C⁺] m/z_{theoretical}=342; m/zₑₓₚₑᵣᵢₘₑₙₜₐₗ=342.

2.2. Peptide Synthesis Supported on Onium Salt—Reverse Route

2.2.1. Grafting of the First Amino Acid.

2.2.2.1. Grafting of Isonipecotic Acid

General procedure 3 for the grafting of isonipecotic acid.

1.0 eq. of onium salt carrying an alcohol function is dissolved in anhydrous acetonitrile. 1.9 eq. of p-nitrophenyl chloroformate and 3.0 eq. of pyridine or NMM are added to the medium which is stirred at AT (Stage 1). The majority of the acetonitrile is then evaporated off then the anhydrous DMF, 3.5 eq. of isonipecotic acid and 3.5 eq. of pyridine or NMM are added to the reaction medium which is stirred at AT (Stage 2). The progress of the reaction is monitored by NMR. The solvents are evaporated off under vacuum. Acetonitrile is added to the residue which is filtered. The solvents of the filtrate are evaporated off under vacuum and the residue is washed three times with ether.

With the Support [HPrTMA]

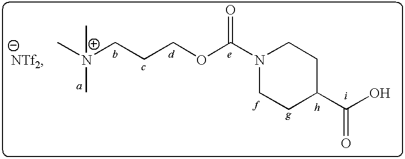

### [HPrTMA-Aiso][NTf₂]

Procedure: cf general procedure 3 using [HPrTMA][NTf₂].

Stage 1: 30 minutes. Stage 2: 24 hours.

The yield by mass is 70%.

10% of [HPrTMA][NTf₂] remains non-grafted (90% conversion).

viscous yellow oil

NMR¹H (200 MHz, acetone d⁶): δ(Hₐ)=3.42 (s, 9H); δ(H_{b})=3.74 (m, 2H); δ(H_{c})=2.36 (m, 2H); δ(H_{d})=4.23 (t, J=6.0, 2H); δ(H_{f})=2.99 (m, 2H); δ(H_{f′})=4.03 (m, 2H); δ(H_{g})=1.58 (m, 2H); δ(H_{g′})=1.91 (m, 2H); δ(Hₕ)=2.56 (m, 1H).

NMR¹³C (50 MHz, acetone d⁶): δ(Cₐ)=54.16 (t, J_{N-C}=3.9); δ(C_{b})=63.02; δ(C_{c})=24.30; δ(C_{d})=65.44; δ(Cₑ)=155.81; δ(C_{f})=44.34; δ(C_{g})=29.27; δ(Cₕ)=41.70; δ(Cᵢ)=177.241; δ(C_{NTf2})=121.39 (q, J_{C-F}=320.9).

HRMS (LSIMS) of (C₁₃H₂₅N₂O₄): [M⁺] m/z_{theoretical}=273.1814; m/zₑₓₚₑᵣᵢₘₑₙₜₐₗ=273.1814.

With the Support [HBuTMA]

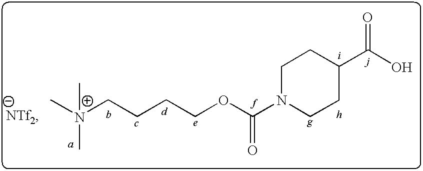

### [HBuTMA-Aiso][NTf₂]

Procedure: cf general procedure 3 using [HBuTMA][NTf₂].

Stage 1: 30 minutes. Stage 2: 24 hours.

The yield by mass is 92%.

20% of [HBuTMA][NTf₂] remains non-grafted (80% conversion).

viscous yellow oil

NMR¹H (200 MHz, acetone d⁶): δ(Hₐ)=3.40 (s, 9H); δ(H_{b})=3.66 (m, 2H); δ(H_{c+d+h+h′})=1.41-2.02 (m, 2H+2H+2H+2H); δ(Hₑ)=4.15 (t, J=6.2, 2H); δ(H_{g})=2.98 (m, 2H); δ(H_{g′})=4.03 (m, 2H); δ(Hᵢ)=2.52 (m, 1H).

NMR¹³C (75 MHz, acetone d⁶): δ(Cₐ)=52.76 (t, J=4.0); δ(C_{b})=63.81; δ(C_{c})=19.56; δ(C_{d})=25.73; δ(Cₑ)=66.22; δ(C_{f})=154.86; δ(C_{g})=43.05; δ(Cₕ)=27.88; δ(Cᵢ)=40.58; δ(Cⱼ)=176.36; δ(C_{NTf2})=120.08 (q, J_{C-F}=321.2).

HRMS (LSIMS) of (C₁₄H₂₇N₂O₄): [M⁺] m/z_{theoretical}=287.1971; m/zₑₓₚₑᵣᵢₘₑₙₜₐₗ=287.1970.

With the support [HHeTMA]

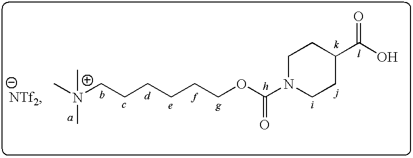

### [HHeTMA-Aiso][NTf₂]

Procedure: cf general procedure 3 using [HHeTMA][NTf₂].

Stage 1: 30 minutes. Stage 2: 24 hours.

The yield is 80%.

20% of [HHeTMA][NTf₂] remains non-grafted (80% conversion).

viscous yellow oil

NMR¹H (300 MHz, acetone d⁶): δ(Hₐ)=3.35 (s, 9H); δ(H_{b})=3.55 (m, 2H); δ(H_{c+d+e+j′})=1.43-1.72 (m, 2H+2H+2H+2H); δ(H_{f+j})=1.82-2.03 (m, 2H+2H); δ(H_{g+i})=3.95-4.07 (m, 2H+2H); δ(H_{i′})=2.96 (m, 2H); δ(Hₖ)=2.54 (m, 1H).

NMR¹³C (75 MHz, acetone d⁶): δ(Cₐ)=52.64; δ(C_{b})=66.50; δ(C_{c})=22.51; δ(C_{d})=25.60; δ(Cₑ)=25.24; δ(C_{f})=27.95; δ(C_{g})=69.09; δ(Cₕ)=155.27; δ(Cᵢ)=43.00; δ(Cⱼ)=28.43; δ(Cₖ)=40.40; δ(Cₗ)=176.13; δ(C_{NTf2})=119.99 (q, J_{C-F}=321.2).

HRMS (LSIMS) of (C₁₆H₃₁N₂O₄): [M⁺] m/z_{theoretical}=315.2284; m/zₑₓₚₑᵣᵢₘₑₙₜₐₗ=315.2279.

With the Support [HMPeTMA]

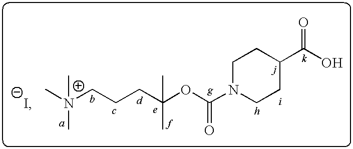

### [HMPeTMA-Aiso][I]

Procedure: cf general procedure 3 using [HMPeTMA][I].

Stage 1: 18 hours. Stage 2: 96 hours.

The yield by mass is 70%.

15% of [HMPeTMA][NTf₂] remains non-grafted (conversion of 85%).

yellow oil

NMR¹H (200 MHz, acetone d⁶): δ(Hₐ)=3.46 (s, 9H); δ(H_{b})=3.70 (m, 2H); δ(H_{c+i+i′})=1.45-1.82 (m, 2H+2H+2H); δ(H_{d})=1.94 (m, 2H); δ(H_{f})=1.51 (s, 6H); δ(H_{h′})=2.98 (m, 2H); δ(Hₕ)=4.02 (m, 2H); δ(Hⱼ)=2.54 (m, 1H).

NMR¹³C (50 MHz, acetone d⁶): δ(Cₐ)=53.24; δ(C_{b})=66.86; δ(C_{c})=17.94; δ(C_{d})=37.05; δ(Cₑ)=82.83; δ(C_{f})=26.04; δ(C_{g})=180.40; δ(Cₕ)=47.05; δ(Cᵢ)=28.17; δ(Cⱼ)=41.38; δ(Cₖ)=156.42.

HRMS (LSIMS) of (C₁₆H₃₁N₂O₄): [M⁺] m/z_{theoretical}=315.2284; m/zₑₓₚₑᵣᵢₘₑₙₜₐₗ=315.2278.

With the Support [HMPhTMA]

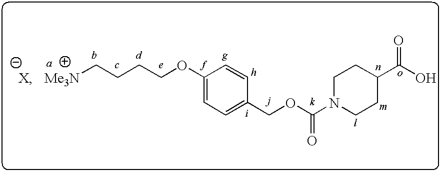

Procedure: cf general procedure 3 using [HMPhBTMA][NTf₂ or PF₆].

Stage 1: 20 minutes. Stage 2: 24 hours.

The yield by mass is 95%.

7 to 8% of [HPrTMA][NTf₂] remains non-grafted and the product is contaminated with 10 to 15% of by-product (determined by NMR) which is totally eliminated during the treatment of the following stage.

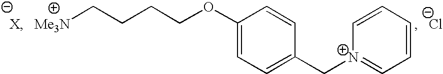

X═NTf₂: [HMPhBTMA-Aiso][NTf₂]

viscous yellow oil

NMR¹H (300 MHz, acetone d₆): δ(Hₐ)=3.40 (s, 9H); δ(H_{b})=3.70 (m, 2H); δ(H_{c+m})=1.85-1.96 (m, 2H+2H); δ(H_{d})=2.17 (m, 2H); δ(Hₑ)=4.11 (t, J=6.0, 2H); δ(H_{g})=6.93 (d, J=8.6, 2H); δ(Hₕ)=7.34 (d, J=8.6, 2H); δ(Hᵢ)=5.04 (s, 2H); δ(H₁)=2.95 (m, 2H); δ(H_{1′})=4.01 (m, 2H); δ(H_{m′})=1.52 (m, 2H); δ(Hₙ)=2.50 (m, 1H).

NMR¹³C (75 MHz, acetone d₆): δ(Cₐ)=52.73 (t, J_{N-C}=3.7); δ(C_{b})=66.25; δ(C_{c})=19.77; δ(C_{d})=25.81; δ(Cₑ)=66.82; δ(C_{f})=158.75; δ(C_{g})=114.24; δ(Cₕ)=129.59; δ(Cᵢ)=129.49; δ(Cⱼ)=66.31; δ(Cₖ)=154.96; δ(Cₗ)=43.23; δ(Cₘ)=29.91; δ(Cₙ)=41.08; δ(Cₒ)=176.77; δ(C_{NTf2})=120.08 (q, J_{C-F}=321.3).

HRMS (ESI) of (C₂ᵢH₃₃N₂O₅): [C⁺] m/z_{theoretical}=393.2390; m/zₑₓₚₑᵣᵢₘₑₙₜₐₗ=393.2390.

### X═PF₆: [HMPhBTMA-Aiso][PF₆]

NMR¹H (300 MHz, acetone d₆): δ(Hₐ)=3.39 (s, 9H); δ(H_{b})=3.68 (m, 2H); δ(H_{c+m})=1.80-1.98 (m, 2H+2H); δ(H_{d})=2.14 (m, 2H); δ(Hₑ)=4.11 (t, J=6.1, 2H); δ(H_{g})=6.93 (d, J=8.6, 2H); δ(Hₕ)=7.34 (d, J=8.5, 2H); δ(Hⱼ)=5.04 (s, 2H); δ(Hₗ)=2.94 (m, 2H); δ(H_{l′})=4.02 (m, 2H); δ(H_{m′})=1.52 (m, 2H); δ(Hₙ)=2.48 (m, 1H).

NMR¹³C (75 MHz, acetone d₆): δ(Cₐ)=52.68 (t, J_{N-C}=4.0); δ(C_{b})=67.03; δ(C_{c})=19.73; δ(C_{d})=25.83; δ(Cₑ)=66.83; δ(C_{f})=158.75; δ(C_{g})=114.42; δ(Cₕ)=129.61; δ(Cᵢ)=129.56; δ(Cⱼ)=66.21; δ(Cₖ)=154.87; δ(Cₗ)=43.22; δ(Cₘ)=28.28; δ(Cₙ)=41.04; δ(Cₒ)=176.26.

2.2.2.2. Grafting of other amino acids)=

With Aminomethyl Esters

General procedure 3′ for the grafting of the amino ester.

1.0 eq. of [HMPhBTMA][PF₆] is dissolved in anhydrous acetonitrile. 2.0 eq. of p-nitrophenyl chloroformate and 3.0 eq. of NMM is added to the medium which is stirred at AT (Stage 1). The acetonitrile is then evaporated off then the anhydrous DMF, 3.5 eq. of methyl amino ester and 3.5 eq. of NMM are added to the reaction medium which is stirred at AT (Stage 2). The progress of the reaction is monitored by NMR. The solvents are then evaporated off under vacuum. Acetonitrile is added to the residue which is filtered. The solvents of the filtrate are evaporated off under vacuum. The residue is washed three times with ether then dissolved in DCM. This organic phase is extracted three times with water, three times with an aqueous solution of HPF₆ (1<pH<2) then it is dried over sodium sulphate. The DCM is then evaporated off.

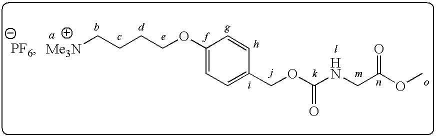

[HMPhBTMA-Gly-OMe][PF₆]

Procedure: cf general procedure 3′ using [HMPhBTMA][PF₆] and Gly-OMe.

Stage 1: 10 minutes. Stage 2: 3 hours.

The yield is 98%. No trace of free [HMPhBTMA][PF₆] is observed with NMR.

viscous yellow oil

NMR¹H (300 MHz, acetone d₆): δ(Hₐ)=3.35 (s, 9H); δ(H_{b+o})=3.58-3.70 (m, 2H+3H); δ(H_{c})=1.91 (m, 2H); δ(H_{d})=2.11 (m, 2H); δ(Hₑ)=4.10 (t, J=6.1, 2H); δ(H_{g})=6.94 (d, J=8.7, 2H); δ(Hₕ)=7.33 (d, J=8.6, 2H); δ(Hⱼ)=5.03 (s, 2H); δ(Hₗ)=6.62 (m, 1H); δ(Hₘ)=3.90 (d, J=6.2, 2H).

NMR¹³C (75 MHz, acetone d₆): δ(Cₐ)=53.60; δ(C_{b})=67.15; δ(C_{c})=20.67; δ(C_{d})=26.72; δ(Cₑ)=67.68; δ(C_{f})=159.66; δ(C_{g})=115.22; δ(Cₕ)=130.61; δ(Cᵢ)=130.28; δ(Cⱼ)=66.66; δ(Cₖ)=157.55; δ(Cₘ)=43.04; δ(Cₙ)=171.38; δ(Cₒ)=52.13.

HRMS (ESI) of (C₁₈H₂₉N₂O₅): [C⁺] m/z_{theoretical}=353.2076; m/zₑₓₚₑᵣᵢₘₑₙₜₐₗ=353.2066.

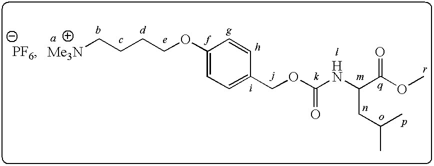

### [HMPhBTMA-Leu-OMe][PF₆]

Procedure: cf general procedure 3′ using [HMPhBTMA][PF₆] and Leu-OMe.

Stage 1: 10 minutes. Stage 2: 3 hours. The yield is 95%. No trace of free [HMPhBTMA][PF₆] is observed with NMR.

viscous yellow oil

NMR¹H (300 MHz, acetone d₆): δ(Hₐ)=3.39 (s, 9H); δ(H_{b+r})=3.66-3.71 (m, 2H+3H); δ(H_{c})=1.92 (m, 2H); δ(H_{d})=2.17 (m, 2H); δ(Hₑ)=4.11 (t, J=6.0, 2H); δ(H_{g})=6.93 (d, J=8.7, 2H); δ(Hₕ)=7.31 (d, J=8.6, 2H); δ(Hⱼ)=5.01 (s, 2H); δ(H₁)=6.60 (m, 1H); δ(Hₘ)=4.11 (m, 1H); δ(H_{n+n′})=1.49-1.66 (m, 1H+1H); δ(Hₒ)=1.73 (m, 1H); δ(Hₚ)=0.93 (d, J=7.4, 6H).

NMR¹³C (75 MHz, acetone d₆): δ(Cₐ)=52.70 (t, J_{N-C}=4.0); δ(C_{b})=66.25 (t, J_{N-C}=2.9); δ(C_{c})=19.77; δ(C_{d})=25.86; δ(Cₑ)=66.79; δ(C_{f})=158.76; δ(C_{g})=114.32; =129.65; δ(Cᵢ)=129.38; δ(Cⱼ)=65.68; δ(Cₖ)=156.32; δ(Cₘ)=52.52; δ(Cₙ)=40.48; δ(Cₒ)=24.52; δ(Cₚ)=20.81; δ(C_{p′})=22.34; δ(C_{q})=173.28; δ(Cᵣ)=51.38.

HRMS (ESI) of (C₂₂H₃₇N₂O₅): [C⁺] m/z_{theoretical}=409.2702; m/zₑₓₚₑᵣᵢₘₑₙₜₐₗ=409.2700.

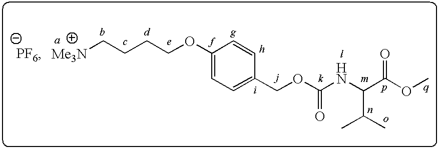

### [HMPhBTMA-Val-OMe][PF₆]

Procedure: cf general procedure 3′ using [HMPhBTMA][PF₆] and Val-OMe.

Stage 1: 10 minutes. Stage 2: 3 hours. The yield is 88%.

No trace of free [HMPhBTMA][PF₆] is observed with NMR.

viscous yellow oil

NMR¹H (300 MHz, acetone d₆): δ(Hₐ)=3.38 (s, 9H); δ(H_{b+q})=3.61-3.73 (m, 2H+3H); δ(H_{c})=1.92 (m, 2H); δ(H_{d+n})=2.08-2.22 (m, 2H+1H); δ(Hₑ₊ₘ)=4.06-4.17 (m, 2H+1H); δ(H_{g})=6.93 (d, J=8.7, 2H); δ(Hₕ)=7.32 (d, J=8.6, 2H); δ(Hⱼ)=5.01 (s, 2H); δ(H₁)=6.44 (m, 1H); δ(Hₒ)=0.94 (d, J=4.8, 3H); δ(Hₒ)=0.97 (d, J=4.8, 3H).

NMR¹³C (75 MHz, acetone d₆): δ(Cₐ)=52.60 (t, J_{N-C}=3.8); δ(C_{b})=66.16; δ(C_{c})=19.63; δ(C_{d})=25.81; δ(Cₑ)=66.88; δ(C_{f})=158.81; δ(C_{g})=114.47; δ(Cₕ)=129.70; δ(Cᵢ)=129.24; δ(Cⱼ)=65.93; δ(Cₖ)=156.62; δ(Cₘ)=59.76; δ(Cₙ)=30.56; δ(Cₒ)=17.54; δ(Cₒ)=18.63; δ(Cₚ)=172.45; δ(C_{q})=51.48.

HRMS (ESI) of (C₂₁H₃₅N₂O₅): [C⁺]m/z_{theoretical}=395.2546; m/zₑₓₚₑᵣᵢₘₑₙₜₐₗ=395.2536.

LRMS (ESI) of (C₄₂H₇₀N₄O₁₀, PF₆): [2C⁺, ⁻PF₆]m/z_{theoretical}=935; m/zₑₓₚₑᵣᵢₘₑₙₜₐₗ=935.

With t-butyl Amino Esters

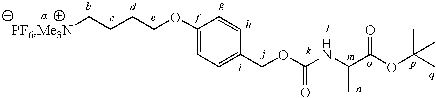

[HMPhBTMA-Ala-OtBu][PF₆]

Procedure: cf general procedure 3′ using [HMPhBTMA][PF₆] and Ala-OtBu.

Stage 1: 10 minutes. Stage 2: 3 hours. The yield by mass is 84%. 3% free [HMPhBTMA][PF₆] contaminates the product (determined by NMR).

viscous yellow oil

NMR¹H (300 MHz, acetone d₆): δ(Hₐ)=3.36 (s, 9H); δ(H_{b})=3.66 (m, 2H); δ(H_{c})=1.91 (m, 2H); δ(H_{d})=2.14 (m, 2H); δ(Hₑ₊ₘ)=4.03-4.17 (m, 2H+1H); δ(H_{g})=6.93 (d, J=8.6, 2H); δ(Hₕ)=7.32 (d, J=8.6, 2H); δ(Hⱼ)=5.00 (s, 2H); δ(H₁)=6.48 (m, 1H); δ(Hₙ)=1.35 (d, J=7.3, 3H); δ(H_{q})=1.44 (s, 9H).

NMR¹³C (75 MHz, acetone d₆): δ(Cₐ)=52.67 (t, J_{N-C}=4.1); δ(C_{b})=66.22 (t, J_{N-C}=2.9); δ(C_{c})=19.75; δ(C_{d})=25.83; δ(Cₑ)=66.80; δ(C_{f})=158.75; δ(C_{g})=114.31; δ(Cₕ)=129.72; δ(Cᵢ)=129.43; δ(Cⱼ)=65.56; δ(Cₖ)=155.95; δ(Cₘ)=50.31; δ(Cₙ)=17.18; δ(Cₒ)=172.08; δ(Cₚ)=80.54; δ(C_{q})=27.23.

HRMS (ESI) of (C₂₂H₃₇N₂O₅): [C⁺] m/z_{theoretical}=409.2702; m/zₑₓₚₑᵣᵢₘₑₙₜₐₗ=409.2690.

With Tri-Terbutoxysilyl Amino Esters

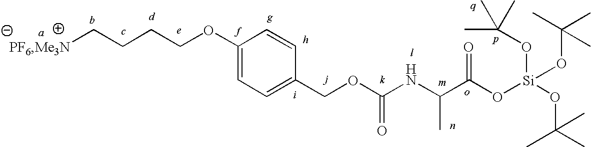

### [HMPhBTMA-Ala-OSil][PF₆]

Procedure: cf general procedure 3′ using [HMPhBTMA][PF₆] and Ala-Osil replacing washing with ether by washing with distilled heptane.

Stage 1: 10 minutes. Stage 2: 3 hours.

The yield is 95%. No trace of free [HMPhBTMA][PF₆] is observed with NMR.

viscous yellow oil

NMR¹H (300 MHz, acetone d₆): δ(Hₐ)=3.40 (s, 9H); δ(H_{b})=3.71 (m, 2H); δ(H_{c})=1.91 (m, 2H); δ(H_{d})=2.14 (m, 2H); δ(Hₑ₊ₘ)=4.05-4.30 (m, 2H+1H); δ(H_{g})=6.92 (d, J=8.6, 2H); δ(Hₕ)=7.32 (d, J=8.6, 2H); δ(Hⱼ)=5.00 (m, 2H); δ(Hᵢᵢ)=1.43 (d, J=7.4, 3H);

δ(H_{q})=1.36 (s, 27H).

NMR¹³C (75 MHz, acetone d₆): δ(Cₐ)=52.76 (t, J_{N-C}=4.0); δ(C_{b})=66.16; δ(C_{c})=19.75; δ(C_{d})=25.88; δ(Cₑ)=66.87; δ(C_{f})=158.78; δ(C_{g})=114.38; δ(Cₕ)=129.74; δ(Cᵢ)=129.33; δ(Cⱼ)=65.66; δ(Cₖ)=155.95; δ(Cₘ)=50.76; δ(Cₙ)=16.84; δ(Cₒ)=170.10; δ(Cₚ)=73.82; δ(C_{q})=30.89.

HRMS (ESI) of (C₃₀H₅₅N₂O₈Si): [C⁺] m/z_{theoretical}=599.3728; m/Zₑₓₚₑᵣᵢₘₑₙₜₐₗ=599.3733.

2.2.2. Synthesis of Supported Protected Dipeptides.

General procedure 4 for reverse route peptide coupling:

1.0 eq. of supported peptide is dissolved in acetonitrile then 1.5 eq. of TEA, carbodiimide (DCC, DIC or EDC.HCl), HOBt and amino ester (Gly-OMe, Ala-OMe, Val-OMe, Phe-OMe or Leu-OMe) are added. The medium is stirred for 2 hours at AT.

If the carbodiimide used is DCC, the reaction medium is filtered (DCU is not very soluble in acetonitrile) then the acetonitrile is evaporated off.

If DIC or EDC.HCl is used, the acetonitrile is evaporated directly.

The residue obtained is then washed with ether.
X═NTf₂ Chromatography on a neutral alumina column is carried out with the eluent DCM/MeOH 1%.
X═PF₆ The residue is dissolved in dichloromethane then the phase is washed three times with water then three times with an aqueous solution of HPF₆ (1<pH<2) before being dried over sodium sulphate then filtered. The dichloromethane is evaporated off.

With the Support [HBuTMA]

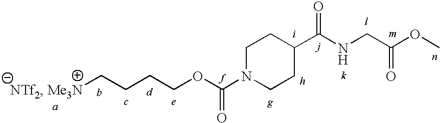

### [HBuTMA-Aiso-Gly-OMe][NTf₂]

Procedure: cf general procedure 4 using [HBuTMA-Aiso][NTf₂] and Gly-OMe.

The yield is 32% (partial loss of [HBuTMA-Aiso-Gly-OMe][NTf₂] during aqueous washing)

viscous yellow oil

NMR¹H (200 MHz, acetone d⁶): δ(Hₐ)=3.40 (s, 9H); δ(H_{b})=3.67 (m, 2H); δ(H_{c+d+h+h′})=1.21-1.88 (m, 2H+2H+2H+2H); δ(H_{e+g})=4.03-4.22 (m, 2H+2H); δ(H_{g′})=2.88 (m, 2H); δ(Hᵢ)=2.54 (m, 1H); δ(Hₖ)=6.87 (m, 1H); δ(Hₗ)=3.96 (d, J=5.9, 2H); δ(Hₙ)=3.70 (s, 3H).

NMR¹³C (75 MHz, acetone d⁶): δ(Cₐ)=53.70 (t, J_{C-N}=4.0); δ(C_{b})=67.11; δ(C_{c})=20.44; δ(C_{d})=25.51; δ(Cₑ)=64.51; δ(C_{f})=155.64; δ(C_{g})=44.05; δ(Cₕ)=30.34; =42.74; δ(Cⱼ)=175.30; δ(Cₗ)=41.36; δ(Cₘ)=171.12; δ(Cₙ)=52.06; δ(C_{NTf2})=121.03 (q, J_{C-F}=321.5).

HRMS (LSIMS) of (C₂₄H₃₈N₃O₆): [M⁺] m/z_{theoretical}=464.2761; m/zₑₓₚₑᵣᵢₘₑₙₜₐₗ=464.2765.

With the Support [HHeTMA]

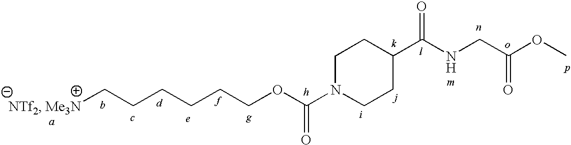

[HHeTMA-Aiso-Gly-OMe][NTf₂]

Procedure: cf general procedure 4 using [HHeTMA-Aiso][NTf₂] and Gly-OMe. The yield is 46% (partial loss of [HHeTMA-Aiso-Gly-OMe][NTf₂] during aqueous washing)

viscous yellow oil

NMR¹H (300 MHz, acetone d⁶): δ(Hₐ)=3.33 (s, 9H); δ(H_{b})=3.55 (m, 2H); δ(H_{c+d+e+j′})=1.43-1.72 (m, 2H+2H+2H+2H); δ(H_{f+j})=1.75-2.02 (m, 2H+2H); δ(H_{g+i})=3.98-4.17 (m, 2H+2H); δ(H_{i′})=2.86 (m, 2H); δ(Hₖ)=2.51 (m, 1H); δ(Hₘ)=7.48 (m, 1H); δ(Hₙ)=3.94 (d, J=5.9, 2H); δ(Hₚ)=3.67 (s, 3H).

NMR¹³C (75 MHz, acetone d⁶): δ(Cₐ)=52.74 (t, J_{C-N}=4.1); δ(C_{b})=64.55; δ(C_{c})=22.59; δ(C_{d})=25.69; δ(Cₑ)=25.31; δ(C_{f})=28.34; δ(C_{g})=66.55; δ(Cₕ)=154.95; δ(Cᵢ)=43.15; δ(Cⱼ)=28.60; δ(Cₖ)=40.55; δ(Cₗ)=174.70; δ(Cₙ)=41.95; δ(Cₒ)=170.22; δ(Cₚ)=51.24; δ(C_{NTf2})=120.09 (q, J_{C-F}=321.1).

HRMS (LSIMS) of (C₁₉H₃₆N₃O₅): [M⁺] m/z_{theoretical}=386.2655; m/zₑₓₚₑᵣᵢₘₑₙₜₐₗ=386.2653.

With the support [HMPhTMA]

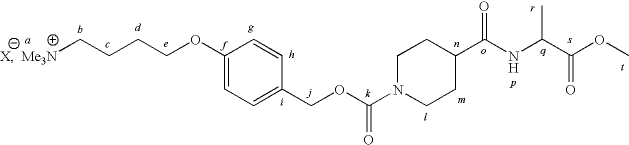

### X═PF₆: [HMPhBTMA-Aiso-Ala-OMe][PF₆]

Procedure: cf general procedure 4 using [HMPhBTMA-Aiso][PF₆] and Ala-OMe. The yield is 85%.

viscous yellow oil

NMR¹H (300 MHz, acetone d₆): δ(Hₐ)=3.38 (s, 9H); δ(H_{b})=3.68 (m, 2H); δ(H_{c})=1.92 (m, 2H); δ(H_{d})=2.16 (m, 2H); δ(Hₑ₊ₗ)=4.02-4.16 (m, 2H+2H); δ(H_{g})=6.94 (d, J=8.7, 2H); δ(Hₕ)=7.34 (d, J=8.6, 2H); δ(Hⱼ)=5.04 (s, 2H); δ(H_{l′})=2.86 (m, 2H); δ(Hₘ)=1.58 (m, 2H); δ(H_{m′})=1.75 (m, 2H); δ(Hₙ)=2.48 (m, 1H); δ(Hₚ)=7.43 (m, 1H); δ(H_{q})=4.41 (m, 1H); δ(Hᵣ)=1.34 (d, J=7.3, 3H); δ(Hₜ)=3.66 (s, 3H).

NMR¹³C (75 MHz, acetone d₆): δ(Cₐ)=52.69 (t, J_{N-C}=3.9); δ(C_{b})=66.17; δ(C_{c})=19.78; δ(C_{d})=25.87; δ(Cₑ)=66.80; δ(C_{f})=158.75; δ(C_{g})=114.38; δ(Cₕ)=129.65; δ(Cᵢ)=129.58; δ(Cⱼ)=66.17; δ(Cₖ)=154.79; δ(Cₗ)=43.19; δ(Cₘ)=28.30; δ(Cₙ)=41.85; δ(Cₒ)=173.98; δ(C_{q})=47.80; δ(Cᵣ)=16.81; δ(Cₛ)=173.12; δ(Cₜ)=51.38.

### X═NTf₂: [HMPhBTMA-Aiso-Ala-OMe][NTf₂]

Procedure: cf general procedure 4 using [HMPhBTMA-Aiso][NTf₂] and Ala-OMe.

The yield is 55%.

viscous yellow oil

NMR¹H (300 MHz, acetone d₆): δ(Hₐ)=3.41 (s, 9H); δ(H_{b})=3.71 (m, 2H); δ(H_{c+m})=1.72-1.97 (m, 2H+2H); δ(H_{d})=2.18 (m, 2H); δ(Hₑ₊ₗ)=4.04-4.16 (m, 2H+2H); δ(H_{g})=6.93 (d, J=8.7, 2H); δ(Hₕ₊ₚ)=7.33-7.36 (m, 2H+1H); δ(Hⱼ)=5.04 (s, 2H); δ(H_{l′})=2.85 (m, 2H); δ(H_{m′})=1.58 (m, 2H); δ(Hₙ)=2.48 (m, 1H); δ(H_{q})=4.42 (m, 1H); δ(Hᵣ)=1.34 (d, J=7.3, 3H); δ(Hₜ)=3.67 (s, 3H).

NMR¹³C (75 MHz, acetone d₆): δ(Cₐ)=52.78 (t, J_{N-C}=4.1); δ(C_{b})=66.30; δ(C_{c})=19.84; δ(C_{d})=25.84; δ(Cₑ)=66.77; δ(C_{f})=158.73; δ(C_{g})=114.36; δ(Cₕ₊ᵢ)=129.63; δ(Cⱼ)=66.17; δ(Cₖ)=154.80; δ(Cₗ)=43.18; δ(Cₘ)=28.30; δ(Cₙ)=41.88; δ(Cₒ)=174.01; δ(C_{q})=47.81; δ(Cᵣ)=16.81; δ(Cₛ)=173.07; δ(Cₜ)=51.37; δ(C_{NTf2})=120.12 (q, J_{C-F}=321.4).

HRMS (ESI) of (C₂₅H₄₀N₃O₆): [C⁺] m/z=_{theoretical}=478.2917: m/zₑₓₚₑᵣᵢₘₑₙₜₐₗ=478.2918.

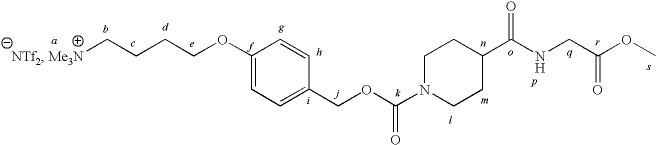

### [HMPhBTMA-Aiso-Gly-OMe][NTf₂]

Procedure: cf general procedure 4 using [HMPhBTMA-Aiso][NTf₂] and Gly-OMe.

The yield is 74%.

viscous yellow oil

NMR¹H (300 MHz, acetone d₆): δ(Hₐ)=3.40 (s, 9H); δ(H_{b})=3.70 (m, 2H); δ(H_{c+m})=1.75-1.98 (m, 2H+2H); δ(H_{d})=2.18 (m, 2H); δ(Hₑ₊ₗ)=4.04-4.20 (m, 2H+2H); δ(H_{g})=6.93 (d, J=8.7, 2H); δ(Hₕ)=7.34 (d, J=8.6, 2H); δ(H_{J})=5.04 (s, 2H); δ(Hᵣ)=2.90 (m, 2H); δ(H_{m′})=1.60 (m, 2H); δ(Hₙ)=2.53 (m, 1H); δ(Hₚ)=7.43 (m, 1H); δ(H_{q})=3.94 (d, J=5.9, 2H); δ(Hₛ)=3.67 (s, 3H).

NMR¹³C (75 MHz, acetone d₆): δ(Cₐ)=52.80 (t, J_{N-C}=4.1); δ(C_{b})=66.36; δ(C_{c})=19.87; δ(C_{d})=25.83; δ(Cₑ)=66.76; δ(C_{f})=158.72; δ(C_{g})=114.35; δ(Cₕ₊₁)=129.64; δ(Cⱼ)=66.13; δ(Cₖ)=154.76; δ(Cₗ)=40.50; δ(Cₘ)=28.70; δ(Cₙ)=41.91; δ(Cₒ)=174.41; δ(C_{q})=40.50; δ(Cᵣ)=170.23; δ(Cₛ)=51.18; δ(C_{NTf2})=120.14 (q, J_{C-F}=321.5).

HRMS (ESI) of (C₂₄H₃₈N₃O₆): [M⁺] m/z_{theoretical}=464.2761; m/zₑₓₚₑᵣᵢₘₑₙₜₐₗ=464.2765.

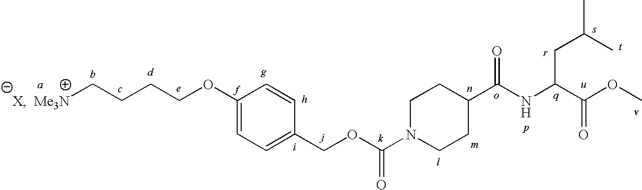

### X═NTf₂: [HMPhBTMA-Aiso-Leu-OMe][NTf₂]

Procedure: cf general procedure 4 using [HMPhBTMA-Aiso][NTf₂] and Leu-OMe.

The yield is 65%.

viscous yellow oil

NMR¹H (300 MHz, acetone d₆): δ(Hₐ)=3.39 (s, 9H); δ(H_{b})=3.70 (m, 2H); δ(H_{c})=1.92 (m, 2H); δ(H_{d})=2.17 (m, 2H); δ(Hₑ₊ₗ)=4.03-4.17 (m, 2H+2H); δ(H_{g})=6.93 (d, J=8.6, 2H); δ(Hₕ₊ₚ)=7.32-7.35 (m, 2H+1H); δ(Hⱼ)=5.04 (s, 2H); δ(H_{l′})=2.87 (m, 2H); δ(H_{m+r+r′+s})=1.47-1.69 (m, 2H+1H+1H+1H); δ(H_{m′})=1.75 (m, 2H); δ(Hₙ)=2.50 (m, 1H); δ(H_{q})=4.49 (m, 1H); δ(Hₜ)=0.90 (d, J=6.4, 3H); δ(H_{t′})=0.93 (d, J=6.4, 3H); δ(Hᵥ)=3.66 (s, 3H).

NMR¹³C (75 MHz, acetone d₆): δ(Cₐ)=52.76 (t, J_{N-C}=3.9); δ(C_{b})=66.33; δ(C_{c})=19.82; δ(C_{d})=25.84; δ(Cₑ)=66.80; δ(C_{f})=158.75; δ(C_{g})=114.41; δ(Cₕ)=129.60; δ(Cᵢ)=129.53; δ(Cⱼ)=66.25; δ(Cₖ)=154.85; δ(Cₗ)=43.20; δ(Cₘ)=28.70; δ(Cₙ)=41.95;

δ(Cₒ)=174.39; δ(C_{q})=50.55; δ(Cᵣ)=40.39; δ(Cₛ)=24.64; δ(Cₜ)=20.97; δ(C_{t′})=22.39; δ(Cₙ)=173.08; δ(Cᵥ)=51.40; δ(C_{NTf2})=120.10 (q, J_{C-F}=321.3).

HRMS (ESI) of (C₂₈H₄₆N₃O₆): [C⁺] m/z_{theoretical}=520.3386; m/zₑₓₚₑᵣᵢₘₑₙₜₐₗ=520.3386.

### X═PF₆: [HMPhBTMA-Aiso-Leu-OMe][PF₆]

Procedure: cf general procedure 4 using [HMPhBTMA-Aiso][PF₆] and Leu-OMe. The yield is 85%.

viscous yellow oil

NMR¹H (300 MHz, acetone d₆): δ(Hₐ)=3.38 (s, 9H); δ(H_{b})=3.68 (m, 2H); δ(H_{c}) 1.92 (m, 2H); δ(H_{d})=2.17 (m, 2H); δ(Hₑ₊ᵢ)=4.03-4.15 (m, 2H+2H); δ(H_{g})=6.93 (d, J=8.7, 2H); δ(Hₕ₊ₚ)=7.29-7.36 (m, 2H+1H); δ(Hⱼ)=5.04 (s, 2H); δ(H_{l′})=2.87 (m, 2H); δ(H_{m+r+r′+s})=1.45-1.69 (m, 2H+1H+1H+1H); δ(H_{m′})=1.75 (m, 2H); δ(Hₙ)=2.48 (m, 1H); δ(H_{q})=4.49 (m, 1H); δ(Hₜ)=0.90 (d, J=6.3, 3H); δ(H_{t′})=0.93 (d, J=6.4, 3H); δ(Hᵥ)=3.66 (s, 3H).

NMR¹³C (75 MHz, acetone d₆): δ(Cₐ)=52.56; δ(C_{b})=66.40; δ(C_{c})=19.64; δ(C_{d})=25.82; δ(Cₑ)=66.87; δ(C_{f})=158.76; δ(C_{g})=114.52; δ(Cₕ)=129.62; δ(Cᵢ)=129.41; δ(Cⱼ)=66.12; δ(Cₖ)=154.98; δ(Cₗ)=43.25; δ(Cₘ)=28.57; δ(Cₙ)=41.94; δ(Cₒ)=174.76; δ(C_{q})=50.67; δ(Cᵣ)=40.25; δ(Cₛ)=24.68; δ(Cₜ)=21.08; δ(C_{t′})=22.53; δ(Cᵤ)=173.15; δ(Cᵥ)=51.64.

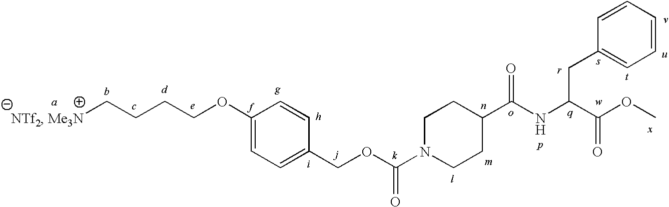

[HMPhBTMA-Aiso-Phe-OMe][NTf₂]

Procedure: cf general procedure 4 using [HMPhBTMA-Aiso][NTf₂] and Phe-OMe. The yield is 65%.

viscous yellow oil

NMR¹H (300 MHz, acetone d₆): δ(Hₐ)=3.36 (s, 9H); δ(H_{b})=3.72 (m, 2H); δ(H_{c})=1.91 (m, 2H); δ(H_{d})=2.15 (m, 2H); δ(Hₑ₊ᵢ)=3.99-4.11 (m, 2H+2H); δ(H_{g})=6.96 (d, J=8.6, 2H); δ(Hₕ₊ₚ₊ₜ₊ᵤ₊ᵥ)=7.04-7.37 (m, 2H+1H+2H+2H+1H); δ(Hⱼ)=5.03 (s, 2H); δ(H₁)=2.81 (m, 2H); δ(Hₘ)=1.48 (m, 2H); δ(H_{m′})=1.68 (m, 2H); δ(Hᵢᵢ)=2.44 (m, 1H); δ(H_{q})=4.71 (m, 1H); δ(H_{r+r′})=2.95-3.21 (m, 1H+1H); δ(Hₓ)=3.67 (s, 3H).

NMR¹³C (75 MHz, acetone d₆): δ(Cₐ)=52.79 (t, J_{N-C}=3.9); δ(C_{b})=66.33; δ(C_{c})=19.85; δ(C_{d})=25.83; δ(Cₑ)=66.79; δ(C_{f})=158.73; δ(C_{g})=114.40; δ(Cₕ)=129.59; δ(Cᵢ)=129.55; δ(Cⱼ)=66.27; δ(Cₖ)=155.84; δ(Cₗ)=43.15; δ(Cₘ)=29.56; δ(Cₙ)=41.89; δ(Cₒ)=174.12; δ(C_{q})=53.47; δ(Cᵣ)=37.34; δ(Cₛ)=137.18; δ(Cₜ)=129.24; δ(Cᵤ)=128.30; δ(Cᵥ)=126.67; δ(C_{w})=171.95; δ(Cₓ)=51.56; δ(C_{NTf2})=120.10 (q, J_{C-F}=321.3).

HRMS (ESI) of (C₃₁H₄₄N₃O₆): m/z_{theoretical}=554.3230; m/zₑₓₚₑᵣᵢₘₑₙₜₐₗ=554.3233.

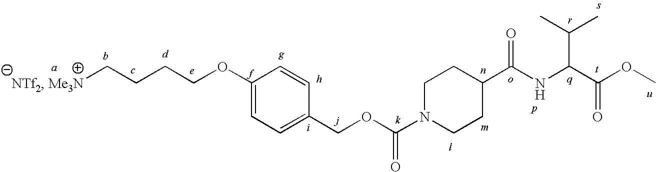

[HMPhBTMA-Aiso-Val-OMe][NTf₂]

Procedure: cf general procedure 4 using [HMPhBTMA-Aiso][NTf₂] and Val-OMe.

The yield is 65%.

viscous yellow oil

NMR¹H (300 MHz, acetone d₆): δ(Hₐ)=3.41 (s, 9H); δ(H_{b})=3.71 (m, 2H); δ(H_{c})=1.93 (m, 2H); δ(H_{d+r})=2.08-2.24 (m, 2H+1H); δ(Hₑ₊ₗ)=4.05-4.18 (m, 2H+2H); δ(H_{g})=6.93 (d, J=8.7, 2H); δ(Hₕ)=7.34 (d, J=8.6, 2H); δ(Hⱼ)=5.04 (s, 2H); δ(H_{l′})=2.87 (m, 2H); δ(Hₘ)=1.60 (m, 2H); δ(H_{m′})=1.78 (m, 2H); δ(Hₙ)=2.56 (m, 1H); δ(Hₚ)=7.34 (m, 1H); δ(H_{q})=4.41 (m, 1H); δ(H_{s+s′})=0.90-0.94 (m, 3H+3H); δ(Hᵤ)=3.69 (s, 3H).

NMR¹³C (75 MHz, acetone d₆): δ(Cₐ)=52.76 (t, J_{N-C}=4.0); δ(C_{b})=66.31; δ(C_{c})=19.82; δ(C_{d})=25.83; δ(Cₑ)=66.80; δ(C_{f})=158.74; δ(C_{g})=114.41; δ(Cₕ)=129.59; δ(Cᵢ)=129.53; δ(Cⱼ)=66.26; δ(Cₖ)=155.87; δ(Cₗ)=43.24; δ(Cₘ)=26.59; δ(Cₙ)=41.95; δ(Cₒ)=174.48; δ(C_{q})=57.41; δ(Cᵣ)=30.54; δ(Cₛ)=17.53; δ(C_{s′})=17.60; δ(Cₜ)=172.12; δ(Cᵤ)=51.27; δ(C_{NTf2})=120.10 (q, J_{C-F}=321.4).

HRMS (ESI) of (C₂₇H₄₄N₃O₆): [C⁺] m/z_{theoretical}=506.3230; m/zₑₓₚₑᵣᵢₘₑₙₜₐₗ=506.3226.

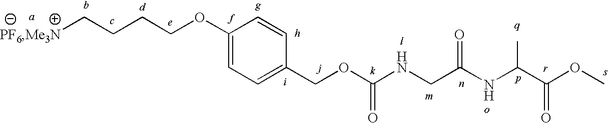

### [HMPhBTMA-Gly-Ala-OMe][PF₆]

Procedure: cf general procedure 5 using [HMPhBTMA-Gly-OMe][PF₆] then 4 using [HMPhBTMA-Gly][PF₆] formed and Ala-OMe.

The yield by mass is 95% (over the two stages).

viscous yellow oil

NMR¹H (300 MHz, acetone d₆): δ(Hₐ)=3.38 (s, 9H); δ(H_{b})=3.68 (m, 2H); δ(H_{c})=1.92 (m, 2H); δ(H_{d})=2.17 (m, 2H); δ(Hₑ)=4.11 (t, J=6.0, 2H); δ(H_{g})=6.93 (d, J=8.7, 2H); δ(Hₕ)=7.33 (d, J=8.6, 2H); δ(Hⱼ)=5.02 (s, 2H); δ(H₁)=6.47 (m, 1H); δ(Hₘ)=3.83 (d, J=5.9, 2H); δ(Hₒ)=7.50 (m, 1H); δ(Hₚ)=4.46 (m, 1H); δ(H_{q})=1.35 (d, J=7.2, 3H); δ(Hₛ)=3.68 (s, 3H).

NMR¹³C (75 MHz, acetone d₆): δ(Cₐ)=52.69 (t, J_{N-C}=4.0); δ(C_{b})=66.24; δ(C_{c})=19.75; δ(C_{d})=25.84; δ(Cₑ)=66.81; δ(C_{f})=158.77; δ(C_{g})=114.36; δ(Cₕ)=129.71; δ(Cᵢ)=129.37; δ(Cⱼ)=66.24; δ(Cₖ)=157.00; δ(Cₘ)=43.75; δ(Cₙ)=172.85; δ(Cₚ)=47.87; δ(C_{q})=17.01; δ(Cᵣ)=168.92; δ(Cₛ)=51.51.

HRMS (ESI) of (C₂₁H₃₄N₃O₆): [C⁺] m/z_{theoretical}=424.2448; m/zₑₓₚₑᵣᵢₘₑₙₜₐₗ=424.2448.

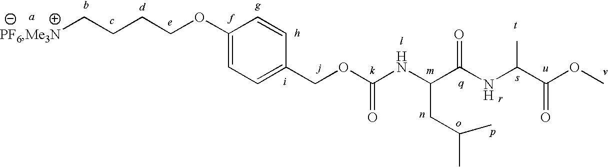

### [HMPhBTMA-Leu-Ala-OMe][PF₆]

Procedure: cf general procedure 5 using [HMPhBTMA-Leu-OMe][PF₆] then 4 using [HMPhBTMA-Leu][PF₆] and Ala-OMe.

The yield is 40% over the two stages.

viscous yellow oil

NMR¹H (300 MHz, acetone d₆): δ(Hₐ)=3.38 (s, 9H); δ(H_{b})=3.68 (m, 2H); δ(H_{c})=1.92 (m, 2H); δ(H_{d})=2.17 (m, 2H); δ(Hₑ)=4.10 (t, J=6.0, 2H); δ(H_{g})=6.93 (d, J=8.6, 2H); δ(Hₕ)=7.32 (d, J=8.6, 2H); δ(Hⱼ)=5.01 (s, 2H); δ(H₁)=6.34 (m, 1H); δ(Hₘ)=4.24 (m, 1H); δ(H_{n+n′})=1.50-1.65 (m, 1H+1H); δ(Hₒ)=1.76 (m, 1H); δ(Hₚ)=0.92 (m, 6H); δ(Hᵣ)=7.58 (m, 1H); δ(Hₛ)=4.43 (m, 1H); δ(Hₜ)=1.35 (d, J=7.3, 3H); δ(Hᵥ)=3.68 (s, 3H).

NMR¹³C (75 MHz, acetone d₆): δ(Cₐ)=52.68 (t, J_{N-C}=4.1); δ(C_{b})=66.23; δ(C_{c})=19.74; δ(C_{d})=25.87; δ(Cₑ)=66.83; δ(C_{f})=158.76; δ(C_{g})=114.35; δ(Cₕ)=129.62; δ(Cᵢ)=129.39; δ(Cⱼ)=65.71; δ(Cₖ)=156.22; δ(Cₘ)=51.49; δ(Cₙ)=41.41; δ(Cₒ)=24.42; δ(Cₚ)=22.58; δ(C_{p′})=21.13; δ(C_{q})=172.25; δ(Cₛ)=47.89; δ(Cᵣ)=16.85; δ(Cᵤ)=172.86; δ(Cᵥ)=51.49.

HRMS (ESI) of (C₂₅H₄₂N₃O₆): [C⁺] m/z_{theoretical}=480.3074; m/zₑₓₚₑᵣᵢₘₑₙₜₐₗ=480.3074.

2.2.3. Deprotection of the Supported Dipeptides.

General procedure 5 for the cleavage of the terminal methyl ester

1.0 eq. of protected supported peptide is dissolved in anhydrous acetonitrile. 2.0 eq. of potassium trimethylsilanolate is added to the medium which is then stirred for 2 hours at AT. The medium is then filtered on celite. The solvents are evaporated off under vacuum and the residue is washed with ether.

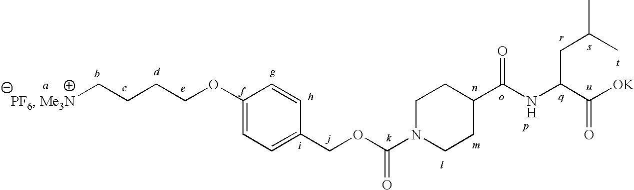

### [HMPhBTMA-Aiso-Leu-OK][PF₆]

Procedure: cf general procedure 5 using [HMPhBTMA-Aiso-Leu-OMe][PF₆].

The yield is 97%.

viscous yellow oil

NMR¹H (200 MHz, acetone d₆): δ(Hₐ)=3.36 (s, 9H); δ(H_{b})=3.66 (m, 2H); δ(H_{c+d+m+m′+r+r′+s})=1.31-2.23 (m, 2H+2H+2H+2H+1H+1H+1H); δ(H_{e+l+q})=3.99-4.29 (m, 2H+2H+1H); δ(H_{g})=6.94 (d, J=8.6, 2H); δ(Hₕ)=7.34 (d, J=8.5, 2H); δ(Hⱼ)=5.04 (s, 2H); δ(H_{l′})=2.84 (m, 2H); δ(Hₙ)=2.59 (m, 1H); δ(Hₚ)=7.89 (m, 1H); δ(Hₜ₊ₑ)=0.80-1.02 (m, 3H+3H).

NMR¹³C (75 MHz, acetone d₆): δ(Cₐ)=52.67; δ(C_{b})=66.20; δ(C_{c})=19.74; δ(C_{d})=25.83; δ(Cₑ)=66.82; δ(C_{f})=158.72; δ(C_{g})=114.42; δ(Cₕ)=129.62; δ(Cᵢ)=129.52; δ(Cⱼ)=66.20; δ(Cₖ)=154.87; δ(Cₗ)=43.36; δ(Cₘ)=29.28; δ(Cₙ)=42.10; δ(Cₒ)=178.24; δ(C_{q})=53.58; δ(Cᵣ)=41.89; δ(Cₛ)=25.13; δ(Cᵣ)=21.59; δ(C_{t′})=23.26; δ(Cᵤ)=174.57.

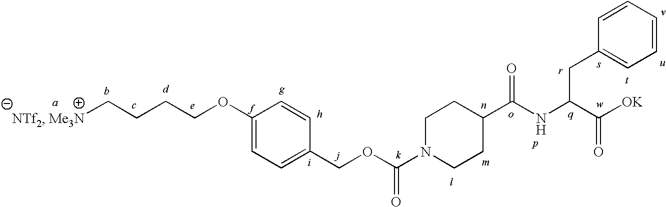

### [HMPhBTMA-Aiso-Phe-OK][NTf₂]

Procedure: cf general procedure 5 using [HMPhBTMA-Aiso-Phe-OMe][NTf₂].

The yield is 95%.

viscous yellow oil

NMR¹H (300 MHz, acetone d₆): δ(Hₐ)=3.36 (s, 9H); δ(H_{b})=3.72 (m, 2H); δ(H_{c})=1.91 (m, 2H); δ(H_{d})=2.15 (m, 2H); δ(Hₑ₊ₗ)=3.99-4.11 (m, 2H+2H); δ(H_{g})=6.96 (d, J=8.6, 2H); δ(Hₕ₊ₚ₊ₜ₊ᵤ₊ᵥ)=7.04-7.37 (m, 2H+1H+2H+2H+1H); δ(Hⱼ)=5.03 (s, 2H); δ(H_{l′})=2.81 (m, 2H); δ(Hₘ)=1.48 (m, 2H); δ(H_{m′})=1.68 (m, 2H); δ(Hₙ)=2.44 (m, 1H); δ(H_{q})=4.71 (m, 1H); δ(H_{r+r′})=2.95-3.21 (m, 1H+1H).

NMR¹³C (75 MHz, acetone d₆): δ(Cₐ)=52.79; δ(C_{b})=66.22; δ(Cₑ)=19.82; δ(C_{d})=25.85; δ(Cₑ)=68.41; δ(C_{f})=158.71; δ(C_{g})=114.41; δ(Cₕ)=129.62; δ(Cᵢ)=129.53; δ(Cⱼ)=66.81; δ(Cₖ)=154.83; δ(Cₗ)=43.27; δ(Cₘ)=29.56; δ(Cₙ)=42.00; δ(Cₒ)=174.01; δ(C_{q})=56.12; δ(Cᵣ)=38.09; δ(Cₛ)=139.78; δ(Cₜ)=129.62; δ(Cᵤ)=127.81; δ(Cᵥ)=125.72; δ(C_{w})=176.48; δ(C_{NTf2})=120.11 (q, J_{C-F}=321.3).

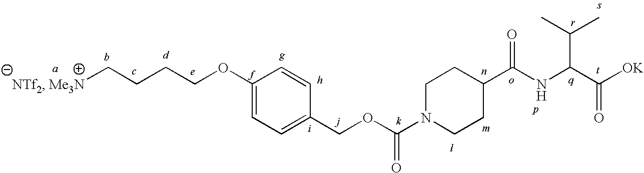

### [HMPhBTMA-Aiso-Val-OK][NTf₂]

Procedure: cf general procedure 5 using [HMPhBTMA-Aiso-Val-OMe][NTf₂].

The yield is 80%.

viscous yellow oil

NMR¹H (300 MHz, acetone d₆): δ(Hₐ)=3.41 (s, 9H); δ(H_{b})=3.71 (m, 2H); δ(H_{c})=1.93 (m, 2H); δ(H_{d+r})=2.08-2.24 (m, 2H+1H); δ(Hₑ₊ₗ)=4.05-4.18 (m, 2H+2H); δ(H_{g})=6.93 (d, J=8.7, 2H); δ(Hₕ)=7.34 (d, J=8.6, 2H); δ(Hⱼ)=5.04 (s, 2H); δ(H_{l′})=2.87 (m, 2H); δ(Hₘ)=1.60 (m, 2H); δ(H_{m′})=1.78 (m, 2H); δ(Hₙ)=2.56 (m, 1H); δ(Hₚ)=7.34 (m, 1H); δ(H_{q})=4.41 (m, 1H); δ(H_{s+s′})=0.90-0.94 (m, 3H+3H).

NMR¹³C (75 MHz, acetone d₆): δ(Cₐ)=52.78; δ(C_{b})=66.18; δ(C_{c})=19.66; δ(C_{d})=25.86; δ(Cₑ)=66.80; δ(C_{f})=158.71; δ(C_{g})=114.40; δ(Cₕ₊ᵢ)=129.60; δ(Cⱼ)=66.31; δ(Cₖ)=154.84; δ(Cₗ)=43.34; δ(Cₘ)=26.59; δ(Cₙ)=42.17; δ(Cₒ)=177.47; δ(C_{q})=60.03; δ(Cᵣ)=30.54; δ(Cₛ)=12.73; δ(C_{s′})=18.08; δ(Cₜ)=174.34; δ(C_{NTf2})=120.10 (q, J_{C-F}=321.4).

2.2.4. Synthesis of Protected Tripeptides.

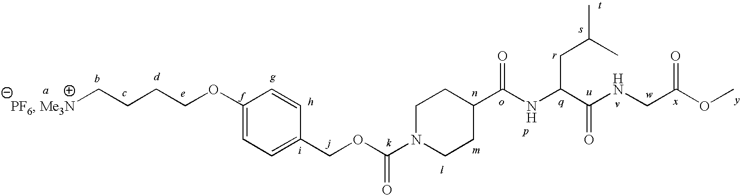

### [HMPhBTMA-Aiso-Leu-Gly-OMe][PF₆]

Procedure: cf general procedure 4 using [HMPhBTMA-Aiso-Leu-OK][PF₆] and Gly-OMe.

The yield is 50%.

viscous yellow oil

NMR¹H (300 MHz, acetone d₆): δ(Hₐ)=3.38 (s, 9H); δ(H_{b+y})=3.58-3.70 (m, 2H+3H); δ(H_{c})=1.91 (m, 2H); δ(H_{d})=2.10 (m, 2H); δ(Hₑ₊ₗ)=4.01-4.18 (m, 2H+2H); δ(H_{g})=6.93 (d, J=8.6, 2H); δ(Hₕ₊ₚ)=7.23-7.37 (m, 2H+1H); δ(Hⱼ)=5.04 (s, 2H); δ(H_{l′})=2.85 (m, 2H); δ(H_{m+m′+s+r+r′})=1.43-1.82 (m, 2H+2H+1H+1H+1H); δ(Hₙ)=2.49 (m, 1H); δ(H_{q})=4.48 (m, 1H); δ(Hₜ)=0.89 (d, J=6.3, 3H); δ(H_{t′})=0.92 (d, J=6.4, 3H); δ(Hᵥ)=7.62 (m, 1H); δ(H_{w})=3.94 (d, J=7.1, 2H).

NMR¹³C (75 MHz, acetone d₆): δ(Cₐ)=52.68 (t, J_{N-C}=4.0); δ(C_{b})=66.22; δ(C_{c})=19.77; δ(C_{d})=25.84; δ(Cₑ)=66.79; δ(C_{f})=158.75; δ(C_{g})=114.37; δ(Cₕ)=129.68; δ(Cᵢ)=129.52; δ(Cⱼ)=66.22; δ(Cₖ)=154.81; δ(Cₗ)=43.18; δ(Cₘ)=28.52; δ(Cₙ)=42.16; δ(Cₒ)=174.41; δ(C_{q})=51.18; δ(Cᵣ)=40.95; δ(Cₛ)=24.52; δ(Cₜ)=21.11; δ(C_{t′})=22.38; δ(Cᵤ)=172.83; δ(C_{w})=40.57; δ(Cₓ)=170.08; δ(C_{y})=51.32.

HRMS (ESI) of (C₃₀H₄₉N₄O₇): [C⁺] m/z_{theoretical}=577.3601; m/zₑₓₚₑᵣᵢₘₑₙₜₐₗ=577.3614.

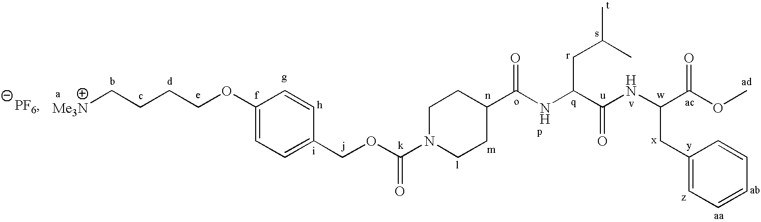

[HMPhBTMA-Aiso-Leu-Phe-OMe][PF₆]

Procedure: cf general procedure 4 using [HMPhBTMA-Aiso-Leu-OK][PF₆] and Phe-OMe. The yield is 92%.

viscous yellow oil

NMR¹H (300 MHz, acetone d₆): δ(Hₐ)=3.36 (s, 9H); δ(H_{b})=3.64 (m, 2H); δ(H_{c})=1.91 (m, 2H); δ(H_{d})=2.10 (m, 2H); δ(Hₑ₊ₗ)=4.03-4.18 (m, 2H+2H); δ(H_{g})=6.93 (d, J=8.5, 2H); δ(H_{h+p+v+z+aa+ab})=7.17-7.51 (m, 2H+1H+1H+2H+2H+1H); δ(Hⱼ)=5.04 (s, 2H); δ(H_{l′})=2.83 (m, 2H); δ(H_{m+m′+s+r+r′})=1.23-1.80 (m, 2H+2H+1H+1H+1H); δ(Hₙ)=2.43 (m, 1H); δ(H_{q})=4.45 (m, 1H); δ(Hₜ)=0.86 (d, J=6.3, 3H); δ(H_{t′})=0.90 (d, J=6.4, 3H); δ(H_{w})=4.69 (m, 1H); δ(H_{x+x′})=2.94-3.23 (m, 1H+1H); δ(H_{ad})=3.67 (s, 3H).

NMR¹³C (75 MHz, acetone d₆): δ(Cₐ)=52.66 (t, J_{N-C}=3.9); δ(C_{b})=66.27; δ(C_{c})=19.75; δ(C_{d})=25.85; δ(Cₑ)=66.82; δ(C_{f})=158.77; δ(C_{g})=114.42; δ(Cₕ)=129.51; δ(Cᵢ)=129.29; δ(Cⱼ)=66.27; δ(Cₖ)=154.83; δ(Cₗ)=43.21; δ(Cₘ)=28.36; δ(Cₙ)=42.05; δ(Cₒ)=174.39; δ(C_{q})=53.51; δ(Cᵣ)=40.71; δ(Cₛ)=24.36; δ(Cₜ)=21.24; δ(C_{t′})=22.54; δ(Cᵤ)=171.62; δ(C_{w})=55.24; δ(Cₓ)=37.29; δ(C_{y})=136.87; δ(C_{z})=128.82; δ(Cₐₐ)=128.35; δ(C_{ab})=126.71; δ(C_{ac})=172.24; δ(C_{ad})=51.56.

HRMS of (C₃₇H₅₅N₄O₇): [C⁺] m/z_{theoretical}=667.4071; m/zₑₓₚₑᵣᵢₘₑₙₜₐₗ=667.4070.

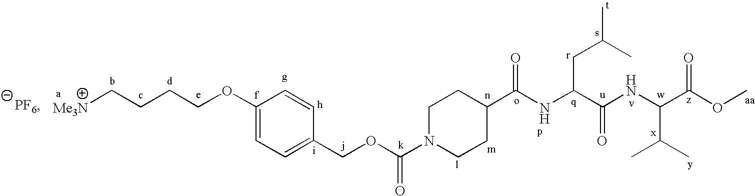

### [HMPhBTMA-Aiso-Leu-Val-OMe][PF₆]

Procedure: cf general procedure 4 using [HMPhBTMA-Aiso-Leu-OK][PF₆] and Val-OMe. The yield is 52%.

viscous yellow oil

NMR¹H (300 MHz, acetone d₆): δ(Hₐ)=3.38 (s, 9H); δ(H_{b})=3.68 (m, 2H); δ(H_{c})=1.92 (m, 2H); δ(H_{d+x})=2.08-2.24 (m, 2H+1H); δ(Hₑ₊ₗ)=4.07-4.18 (m, 2H+2H); δ(H_{g}) 6.93 (d, J=8.6, 2H); δ(Hₕ₊ₚ₊ᵥ)=7.23-7.45 (m, 2H+1H+1H); δ(Hⱼ)=5.04 (s, 2H); δ(H_{l′})=2.84 (m, 2H); δ(H_{m+m′+s+r+r′)=}1.45-1.83 (m, 2H+2H+1H+1H+1H); δ(Hₙ)=2.52 (m, 1H); δ(H_{q})=4.50 (m, 1H); δ(H_{t+t′+y+y′})=0.82-0.97 (m, 3H+3H+3H+3H); δ(H_{w})=4.38 (m, 1H); δ(Hₐₐ)=3.69 (s, 3H).

NMR¹³C (75 MHz, acetone d₆): δ(Cₐ)=52.66 (t, J_{N-C}=4.0); δ(C_{b})=66.25; δ(C_{c})=19.77; δ(C_{d})=25.84; δ(Cₑ)=66.80; δ(C_{f})=158.76; δ(C_{g})=114.39; δ(Cₕ)=129.65; KO=129.50; δ(Cⱼ)=66.25; δ(Cₖ)=154.82; δ(Cₗ)=43.21; δ(Cₘ)=28.57; δ(Cₙ)=42.14; δ(Cₒ)=174.58; δ(C_{q})=51.44; δ(Cᵣ)=40.50; δ(Cₛ)=24.55; δ(Cₜ)=21.21; δ(C_{t′})=21.25; δ(Cᵤ)=172.44; δ(C_{w})=57.25; δ(Cₓ)=30.76; δ(C_{y})=17.40; δ(C_{y′})=18.53; δ(C_{z})=171.80; δ(Cₐₐ)=51.34.

HRMS of (C₃₃H₅₅N₄O₇): [C⁺] m/z_{theoretical}=619.4071; m/zₑₓₚₑᵣᵢₘₑₙₜₐₗ=619.4070.

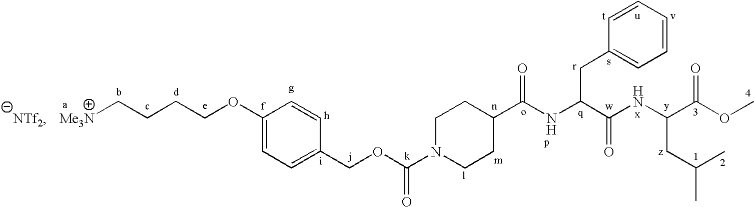

### [HMPhBTMA-Aiso-Phe-Leu-OMe][NTf₂]

Procedure: cf general procedure 4 using [HMPhBTMA-Aiso-Phe-OK][NTf₂] and Leu-OMe.

The yield is 64%.

viscous yellow oil

NMR¹H (300 MHz, acetone d₆): δ(Hₐ)=3.36 (s, 9H); δ(H_{b})=3.70 (m, 2H); δ(H_{c})=1.92 (m, 2H); δ(H_{d})=2.18 (m, 2H); δ(Hₑ₊ₗ)=3.97-4.13 (m, 2H+2H); δ(H_{g})=6.92 (d, J=8.7, 2H); δ(Hₕ)=7.33 (d, J=8.6, 2H); δ(Hⱼ)=5.03 (s, 2H); δ(H_{l′})=2.85 (m, 2H); δ(H_{m+m′+z+z′+1})=1.28-1.78 (m, 2H+2H+1H+1H+1H); δ(Hₙ)=2.43 (m, 1H); δ(Hₚ₊ₜ₊ᵤ₊ᵥ)=7.14-7.26 (m, 1H+2H+2H+1H); δ(H_{q})=4.72 (m, 1H); δ(H_{r+r′})=2.88-3.22 (m, 1H+1H); δ(Hₓ)=7.51 (m, 1H); δ(H_{y})=4.49 (m, 1H); δ(H₂)=0.90 (d, J=6.3, 3H); δ(H_{2′})=0.91 (d, J=6.4, 3H); δ(H₄)=3.68 (s, 3H).

NMR¹³C (75 MHz, acetone d₆): δ(Cₐ)=52.80 (t, J_{N-C}=4.1); δ(C_{b})=66.31; δ(C_{c})=19.86; δ(C_{d})=25.86; δ(Cₑ)=66.78; δ(C_{f})=158.72; δ(C_{g})=114.35; δ(Cₕ)=129.64; δ(Cᵢ)=129.32; δ(Cⱼ)=66.15; δ(Cₖ)=154.74; δ(Cₗ)=43.09; δ(Cₘ)=29.51; δ(Cₙ)=41.98; δ(Cₒ)=174.05; δ(C_{q})=53.84; δ(Cᵣ)=37.62; δ(Cₛ)=137.68; δ(Cₜ)=129.40; δ(Cᵤ)=128.13; δ(Cᵥ)=126.39; δ(C_{w})=172.69; δ(C_{y})=50.62; δ(C_{z})=40.64; δ(C₁)=24.46; δ(C₂)=20.96; δ(C_{2′})=21.04; δ(C₃)=171.29; δ(C₄)=51.45; δ(C_{NTf2})=120.14 (q, J_{C-F}=321.5).

HRMS of (C₃₇H₅₅N₄O₇): [C⁺] m/z_{theoretical}=667.4071; m/zₑₓₚₑᵣᵢₘₑₙₜₐₗ=667.4069.

2.2.5. Cleavage of the Supported Peptides.

General procedure 6 for the cleavage of supported peptides by reverse route by TFA:

1.0 eq. of supported peptide is dissolved in a 10% solution of TFA in anhydrous DCM with a volume of solution such that approximately 10 eq. of TFA with respect to the salt are added. The mixture is stirred 10 minutes at AT then the solvents are evaporated off under vacuum. Dichloromethane and water are added to the residue. The organic phase is washed three times with water. The aqueous phases are combined and the water is evaporated off.

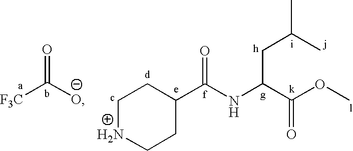

### [Aiso-Leu-OMe][CF₃COO]

Procedure: cf general procedure 6 using [HMPhBTMA-Aiso-Leu-OMe][NTf₂]. The yield is 95%.

colourless oil

NMR¹H (200 MHz, D₂O): δ(H_{c})=2.98 (m, 2H); δ(H_{c′})=3.39 (m, 2H); δ(H_{d+of+i})=1.67-2.04 (m, 2H+2H+1H); δ(Hₑ)=2.61 (m, 1H); δ(H_{g})=4.33 (m, 1H); δ(Hₕ)=1.56 (d, J=6.4, 2H); δ(Hⱼ)=0.80 (d, J=8.9, 3H); δ(H_{j′})=0.82 (d, J=8.9, 3H); δ(Hₗ)=3.65 (s, 3H).

NMR¹³C (75 MHz, D₂O): δ(Cₐ)=116.67 (q, J_{C-F}=289.1); δ(C_{b})=162.89; δ(C_{c})=43.33; δ(C_{d})=25.34; δ(C_{of})=25.12; δ(Cₑ)=39.40; δ(C_{f})=176.86; δ(C_{g})=53.21; δ(Cₕ)=39.66; δ(Cᵢ)=24.75; δ(Cⱼ)=20.82; δ(C^{j′})=22.40; δ(Cₖ)=175.53; δ(Cₗ)=51.75.

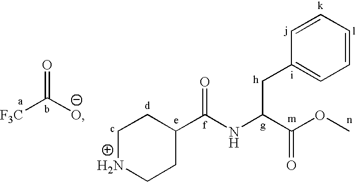

### [Aiso-Phe-OMe][CF₃COO]

Procedure: cf general procedure 6 using [HMPhBTMA-Aiso-Phe-OMe][NTf₂]. The yield is 98%.

colourless oil

NMR¹H (300 MHz, D₂O): δ(H_{c+c′+h+h′})=2.79-3.42 (m, 2H+2H+1H+1H); δ(H_{d+of})=1.41-1.93 (m, 2H+2H); δ(Hₑ)=2.46 (m, 1H); δ(H_{g})=4.65 (dd, J₁=5.5, J₂=4.2, 1H); δ(Hⱼ₊ₖ₊ₗ)=7.14-7.30 (m, 2H+2H+1H); δ(Hₙ)=3.65 (s, 3H).

NMR¹³C (75 MHz, D₂O): δ(Cₐ)=116.30 (q, J_{C-F}=291.5); δ(C_{b})=162.84 (q, J=35.7); δ(C_{c})=42.87; δ(C_{d})=24.91; δ(C_{of})=24.55; δ(Cₑ)=39.19; δ(C_{f})=175.92; δ(C_{g})=53.77; δ(Cₕ)=36.55; δ(Cᵢ)=136.51; δ(Cⱼ)=129.17; δ(Cₖ)=128.69; δ(Cₗ)=127.12; δ(Cₘ)=173.58; δ(Cₙ)=52.95.

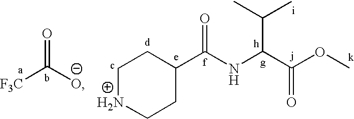

### [Aiso-Val-OMe][CF₃COO]

Procedure: cf general procedure 6 using [HMPhBTMA-Aiso-Val-OMe][NTf₂]. The yield is 98%.

colourless oil

NMR¹H (200 MHz, D₂O): δ(H_{c})=3.01 (m, 2H); δ(H_{c′})=3.43 (m, 2H); δ(H_{d})=1.82 (m, 2H); δ(H_{of})=2.00 (m, 2H); δ(Hₑ)=2.69 (m, 1H); δ(H_{g})=4.22 (d, J=6.1, 1H); δ(Hₕ)=2.12 (m, 1H); δ(Hᵢ)=0.87 (d, J=6.9, 6H); δ(Hₖ)=3.70 (s, 3H).

NMR¹³C (75 MHz, D₂O): δ(Cₐ)=116.22 (q, J=291.3); δ(C_{b})=162.53 (q, J_{C-F}=35.8); δ(C_{c})=43.01; δ(C_{d})=24.81; δ(C_{of})=25.11; δ(Cₑ)=39.26; δ(C_{f})=176.51; δ(C_{g})=58.50; δ(Cₕ)=29.88; δ(Cᵢ)=17.40; δ(C_{i′})=18.20; δ(Cⱼ)=174.08; δ(Cₖ)=52.64.

General procedure 6′ for the cleavage of supported peptides by reverse route by TMSBr

1.0 eq. of supported peptide [HMPhBTMA-AA₁- . . . -AAₙ][PF₆] is dissolved in anhydrous acetonitrile then 1.5 eq. of TMSBr are added. The mixture is stirred for 30 minutes at AT then filtered. The filtrate (peptide) is washed with acetonitrile.

The cleavage under these conditions was tested on [HMPhBTMA-Aiso][PF₆]. The isonipecotic acid was isolated with a yield of 95%.

2.3. Peptide Synthesis Supported on Onium Salt—Direct Route

2.3.1. Grafting of the First Amino Acid

General procedure 7 for the grafting of Fmoc-alanine by esterification

1.0 eq. of onium salt is dissolved in acetonitrile then 1.5 eq. of DCC and 0.1 eq. of DMAP are added. The medium is stirred overnight at AT. The mixture is filtered then the acetonitrile is evaporated off. The residue is washed with ether then dissolved in DCM. This phase is washed twice with one-tenth by volume of a 1N aqueous solution of HCl before being dried over sodium sulphate and filtered. The DCM is then evaporated off.

General procedure 8 for the cleavage of the Fmoc group

The supported peptide having the terminal amine protected by a Fmoc group is dissolved in acetonitrile, then piperidine (10 to 20% by volume) is added. The medium is stirred for 15 minutes at AT before evaporating the solvents. The residue is washed with ether.

With the Support [HPrTMA]

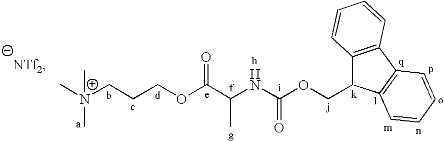

### [Fmoc-Ala-HPrTMA][NTf₂]

Procedure: cf general procedure 7 using [HPrTMA][NTf₂]

The yield is 80%.

viscous yellow oil.

NMR¹H (200 MHz, acetone d⁶): δ(Hₐ)=3.38 (s, 9H); δ(H_{b})=3.71 (m, 2H); δ(H_{c})=2.37 (m, 2H); δ(H_{d±f±j±k})=4.21-4.41 (m, 2H+1H+2H+1H); δ(H_{g})=1.45 (d, J=7.3, 3H); δ(Hₕ)=7.07 (m, 1H); δ(Hₘ)=7.73 (d, J=7.2, 2H); δ(Hₙ₊ₒ)=7.33-7.50 (m, 2H+2H); δ(Hₚ)=7.91 (d, J=6.9, 2H).

NMR¹³C (50 MHz, acetone d⁶): δ(Cₐ)=54.15 (t, J_{N-C}=4.0); δ(C_{b})=62.47; δ(C_{c})=23.97; δ(C_{d})=65.11; δ(Cₑ)=174.14; δ(C_{f})=51.20; δ(C_{g})=17.89; δ(Cᵢ)=157.48; δ(Cⱼ)=67.65; δ(Cₖ)=48.34; δ(Cₗ)=145.39; δ(Cₘ)=126.55; δ(Cₙ)=128.44; δ(Cₒ)=129.06; δ(Cₚ)=121.31; δ(C_{q})=142.51; δ(C_{NTf2})=121.43 (q, J_{C-F}=321.2).

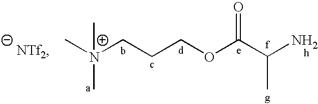

### [Ala-HPrTMA][NTf₂]

Procedure: cf general procedure 8 using [Fmoc-Ala-HPrTMA][NTf₂].

The yield is 97%.

viscous yellow oil

NMR¹H (200 MHz, acetone d⁶): δ(Hₐ)=3.43 (s, 9H); δ(H_{b})=3.72 (m, 2H); δ(H_{c})=2.37 (m, 2H); δ(H_{d})=4.28 (t, J=6.1, 2H); δ(H_{f})=4.37 (q, J=6.7, 1H); δ(H_{g})=1.33 (d, J=6.7, 3H).

NMR¹³C (75 MHz, acetone d⁶): δ(Cₐ)=52.85; δ(C_{b})=61.22; δ(C_{c})=25.98; δ(C_{d})=63.87; δ(Cₑ)=172.82; δ(C_{f})=57.94; δ(C_{g})=20.75; δ(C_{NTf2})=120.01 (q, J_{C-F}=321.1).

HRMS (LSIMS) of (C₉H₂₁N₂O₂): [M⁺] m/z_{theric}=189.1603; m/zₑₓₚₑᵣᵢₘₑₙₜₐₗ=189.1610.

With the Support [HBuTMA]

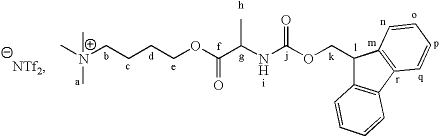

### [Fmoc-Ala-HBuTMA][NTf₂]

Procedure: cf general procedure 7 using [HBuTMA][NTf₂]

The yield is 71%.

viscous yellow oil

NMR¹H (300 MHz, acetone d⁶): δ(Hₐ)=3.34 (s, 9H); δ(H_{b})=3.61 (m, 2H); δ(H_{c})=1.80 (m, 2H); δ(H_{d})=2.05 (m, 2H); δ(H_{e+g+k+l})=4.12-4.42 (m, 2H+1H+2H+1H); δ(Hₕ)=1.43 (d, J=7.3, 3H); δ(Hᵢ)=7.03 (m, 1H); δ(Hₙ)=7.71 (d, J=7.4, 2H); δ(Hₒ₊ₚ)=7.33-7.47 (m, 2H+2H); δ(H_{q})=7.89 (d, J=7.5, 2H).

NMR¹³C (75 MHz, acetone d⁶): δ(Cₐ)=52.72 (t, J_{N-C}=4.0); δ(C_{b})=63.55; δ(C_{c})=19.40; δ(C_{d})=25.26; δ(Cₑ)=66.34; δ(C_{f})=172.94; δ(C_{g})=49.90; δ(Cₕ)=16.74; δ(Cⱼ)=156.15; δ(Cₖ)=66.05; δ(Cₗ)=47.07; δ(Cₘ)=144.10; δ(Cₙ)=125.26; δ(Cₒ)=127.13; δ(Cₚ)=127.76; δ(C_{q})=118.01; δ(Cᵣ)=141.21; δ(C_{NTf2})=120.14 (q, J_{C-F}=321.3).

HRMS (LSIMS) of (C₂₅H₃₃N₂O₄): [M⁺] m/z_{theoretical}=425.2440; m/zₑₓₚₑᵣᵢₘₑₙₜₐₗ=425.2437.

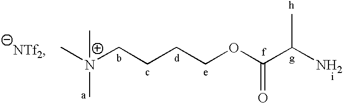

### [Ala-HBuTMA][NTf₂]

Procedure: cf general procedure 8 using [Fmoc-Ala-HBuTMA][NTf₂]. The yield is 87%

viscous yellow oil

NMR¹H (200 MHz, acetone d⁶): δ(Hₐ)=3.41 (s, 9H); δ(H_{b})=3.66 (m, 2H); δ(Hₑ)=1.80 (m, 2H); δ(H_{d})=2.06 (m, 2H); δ(H_{e+g})=4.15-4.28 (m, 2H+1H); δ(Hₕ)=1.30 (d, J=6.7, 3H); δ(Hᵢ)=2.86 (m, 2H).

NMR¹³C (75 MHz, acetone d⁶): δ(Cₐ)=52.73; δ(C_{b})=63.24; δ(Cₑ)=19.48; δ(C_{d})=25.18; δ(Cₑ)=66.58; δ(C_{f})=172.90; δ(C_{g})=58.21; δ(Cₕ)=17.97; δ(C_{NTf2})=120.02 (q, J_{C-F}=321.1).

With the Support [HHeTMA]

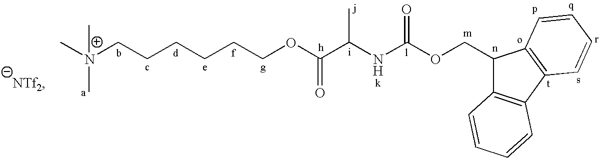

### [Fmoc-Ala-HHeTMA][NTf₂]

Procedure: cf general procedure 7 using [HHeTMA][NTf₂].

The yield is 92%.

viscous yellow oil

NMR¹H (200 MHz, acetone d⁶): δ(Hₐ)=3.35 (s, 9H); δ(H_{b})=3.37 (m, 2H); δ(H_{c+d+e+f})=1.50-2.03 (m, 2H+2H+2H+2H); δ(H_{g})=4.15 (t, J=6.2, 2H); δ(Hᵢ₊ₘ₊ₙ)=4.23-4.40 (m, 1H+2H+1H); δ(Hⱼ)=1.40 (d, J=7.6, 3H); δ(Hₖ)=6.95 (m, 1H); δ(Hₚ)=7.71 (d, J=7.4, 2H); δ(H_{q+r})=7.33-7.50 (m, 2H+2H); δ(Hₛ)=7.92 (d, J=7.1, 2H).

NMR¹³C (75 MHz, acetone d⁶): δ(Cₐ)=52.69 (t, J_{N-C}=4.0); δ(C_{b})=66.45; δ(C_{c})=22.45; δ(C_{d})=25.48; δ(Cₑ)=25.06; δ(C_{f})=28.09; δ(C_{g})=64.42; δ(Cₕ)=172.86; δ(Cᵢ)=49.85; δ(Cⱼ)=16.97; δ(Cₗ)=156.03; δ(Cₘ)=66.49; δ(Cₙ)=47.08; δ(Cₒ)=144.08; δ(Cₚ)=125.27; δ(C_{q})=127.13; δ(Cᵣ)=127.77; δ(Cₛ)=120.01; δ(Cₜ)=141.20; δ(C_{NTf2})=120.13 (q, J_{C-F}=321.3).

HRMS (LSIMS) of (C₂₇H₃₇N₂O₄): [M⁺] m/z_{theoretical}=453.2753; m/zₑₓₚₑᵣᵢₘₑₙₜₐₗ=453.2753.

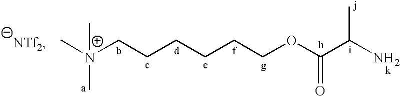

### [Ala-HHeTMA][NTf₂]

Procedure: cf general procedure 8 using [Fmoc-Ala-HHeTMA][NTf₂].

The yield is 90%.

viscous yellow oil

NMR¹H (300 MHz, acetone d⁶): δ(Hₐ)=3.38 (s, 9H); δ(H_{b})=3.59 (m, 2H); δ(H_{c})=1.68 (m, 2H); δ(H_{d+e})=1.42-1.53 (m, 2H+2H); δ(H_{f})=1.98 (m, 2H); δ(H_{g})=4.06 (t, J=6.6, 2H); δ(Hᵢ)=4.20 (q, J=6.7, 1H); δ(Hⱼ)=1.27 (d, J=6.6, 3H); δ(Hₖ)=2.78 (m, 2H).

NMR¹³C (75 MHz, acetone d⁶): δ(Cₐ)=52.65; δ(C_{b})=66.47; δ(C_{c})=22.47; δ(C_{d})=25.47; δ(Cₑ)=25.08; δ(C_{f})=28.07; δ(C_{g})=64.18; δ(Cₕ)=177.12; δ(Cᵢ)=50.34; δ(Cⱼ)=21.05; δ(C_{NTf2})=119.98 (q, J_{C-F}=321.0).

With the Support [HMPhTMA]

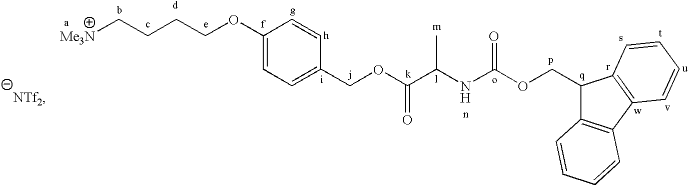

### [Fmoc-Ala-HMPhBTMA][NTf₂]

Procedure: cf general procedure 7 using [HMPhBTMA][NTf₂].

The yield is 88%.

viscous yellow oil

NMR¹H (300 MHz, acetone d₆): δ(Hₐ)=3.40 (s, 9H); δ(H_{b})=3.69 (m, 2H); δ(H_{c})=1.90 (m, 2H); δ(H_{d})=2.16 (m, 2H); δ(Hₑ)=4.06 (t, J=5.9, 2H); δ(H_{g+n})=6.80-6.91 (m, 2H+1H); δ(Hₕ₊ₜ)=7.28-7.37 (m, 2H+2H); δ(Hⱼ)=5.11 (s, 2H); δ(H_{l+p+q})=4.14-4.42 (m, 1H+2H+1H); δ(Hₘ)=1.42 (d, J=7.3, 3H); δ(Hₛ)=7.71 (d, J=7.4, 2H); δ(Hᵤ)=7.43 (m, 2H); δ(Hᵥ)=7.88 (d, J=7.5, 2H).

NMR¹³C (75 MHz, acetone d₆): δ(Cₐ)=52.79 (t, J_{N-C}=4.1); δ(C_{b})=66.27; δ(C_{c})=19.75; δ(C_{d})=25.74; δ(Cₑ)=66.77; δ(C_{f})=158.90; δ(C_{g})=114.43; δ(Cₕ)=129.86; δ(Cᵢ)=128.33; δ(Cⱼ)=66.21; δ(Cₖ)=172.86; δ(Cₗ)=50.00; δ(Cₘ)=16.97; δ(Cₒ)=156.12; δ(Cₚ)=66.41; δ(C_{q})=47.06; δ(Cᵣ)=144.17; δ(Cₛ)=125.31; δ(Cₜ)=126.54; δ(Cᵤ)=127.18; δ(Cᵥ)=120.05; δ(C_{w})=141.21; δ(C_{NTf2})=120.15 (q, J_{C-F}=321.3).

HRMS (ESI) of (C₃₂H₃₉N₂O₅): [C⁺] m/z_{theoretical}=531.2859; m/zₑₓₚₑᵣᵢₘₑₙₜₐₗ=531.2859.

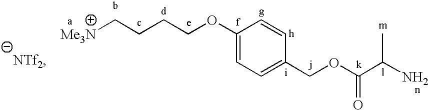

### [Ala-HMPhBTMA][NTf₂]

Procedure: cf general procedure 8 using [Fmoc-Ala-HMPhBTMA][NTf₂].

The yield is 88%.

viscous yellow oil

NMR¹H (300 MHz, acetone d₆): δ(Hₐ)=3.40 (s, 9H); δ(H_{b})=3.70 (m, 2H); δ(H_{c})=1.93 (m, 2H); δ(H_{d})=2.18 (m, 2H); δ(Hₑ)=4.11 (t, J=6.0, 2H); δ(H_{g})=6.93 (d, J=8.6, 2H); δ(Hₕ)=7.32 (d, J=8.6, 2H); δ(Hⱼ)=5.06 (s, 2H); δ(Hₗ)=4.24 (q, J=6.7, 1H); δ(Hₘ)=1.28 (d, J=6.6, 3H); δ(Hₙ)=2.94 (m, 2H).

NMR¹³C (75 MHz, acetone d₆): δ(Cₐ)=52.72 (t, J_{N-C}=3.9); δ(C_{b})=66.29; δ(C_{c})=19.74; δ(C_{d})=25.77; δ(Cₑ)=66.83; δ(C_{f})=158.92; δ(C_{g})=114.46; δ(Cₕ)=129.91; δ(Cᵢ)=128.48; δ(Cⱼ)=65.86; δ(Cₖ)=176.92; δ(Cₗ)=50.42; δ(Cₘ)=20.97; δ(C_{NTf2})=120.06 (q, J_{C-F}=321.2).

With the Support [HTMPPTMA]

General procedure 9 for the grafting of the first amino acid to [HTMPPTMA][PF₆]

[AA¹-HTMPPTMA][PF₆] is synthesized in four stages from {5-[4-(hydroxy-p-tolyl-methyl)-phenoxy]-pentyl}-trimethyl-ammonium bromide [HTMPPTMA][Br]
chlorination of the benzhydryl position
grafting of the Fmoc-amino acid
metathesis of the counter-ion (Br (or optionally Cl)→PF₆)
cleavage of the Fmoc group

1.0 eq. of {5-[4-(hydroxy-p-tolyl-methyl)-phenoxy]-pentyl}-trimethyl-ammonium bromide [HTMPTTMA][Br] is dissolved in anhydrous acetonitrile. 1.5 eq. of thionyl chloride are added dropwise at 0° C. then the medium is stirred for 20 minutes at AT under argon. The solvents are then evaporated off under vacuum. The residue is dissolved in anhydrous acetonitrile then 1.5 eq of Fmoc-amino acid and 1.5 eq. of TEA are added to the medium which is stirred for 30 minutes at AT. The acetonitrile is then evaporated off. The residue is washed with ether and dissolved in acetonitrile. 3.0 eq. of KPF₆ are added to the medium which is stirred for two hours then filtered on celite. The solvents of the filtrate are evaporated off under vacuum and the residue is dissolved in DCM. This phase is washed with three times one-tenth by volume of water then it is dried over sodium sulphate and filtered. The DCM is evaporated off. A mixture of acetonitrile and piperidine (10 to 20%) is added to the residue and the reaction medium is stirred for 15 minutes at AT before evaporating the solvents. The residue is then washed with ether then dissolved in DCM. The organic phase obtained is washed three times with water then dried over sodium sulphate and filtered. The DCM is then evaporated off. The grafting level is greater than 95%.

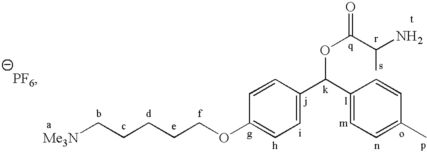

### [Ala-HTMPPTMA][PF₆]

Procedure: cf procedure 9 using Fmoc-Ala.

The yield is 95% over 4 stages.

viscous yellow oil

NMR¹H (300 MHz, acetone d₆): δ(Hₐ)=3.36 (s, 9H); δ(H_{b+r})=3.50-3.63 (m, 2H+1H); δ(H_{c})=1.87 (m, 2H); δ(H_{d})=1.60 (m, 2H); δ(Hₑ)=2.02 (m, 2H); δ(H_{f})=4.03 (t, J=6.2, 2H); δ(Hₕ)=6.90 (d, J=8.5, 2H); δ(Hᵢ₊ₘ₊ₙ)=7.11-7.39 (m, 2H+2H+2H); δ(Hₖ)=6.78 (s, 1H); δ(Hₚ)=2.31 (s, 3H); δ(Hₛ)=1.33 (d, J=6.6, 3H); δ(Hₜ)=2.29 (m, 2H).

NMR¹³C (75 MHz, acetone d₆): δ(Cₐ)=52.69; δ(C_{b})=66.42; δ(C_{c})=22.34; δ(C_{d})=20.25; δ(Cₑ)=28.44; δ(C_{f})=67.23; δ(C_{g})=158.74; δ(Cₕ)=114.34; δ(Cᵢ)=128.35; δ(Cⱼ)=132.99; δ(Cₖ)=76.47; δ(Cₗ)=137.25; δ(Cₘ)=126.57; δ(Cₙ)=129.03; δ(Cₒ)=138.19; δ(Cₚ)=20.13; δ(C_{q})=171.48; δ(Cᵣ)=58.47; δ(Cₛ)=18.06.

HRMS (ESI) of (C₂₅H₃₇N₂O₃): [C⁺] m/z_{theoretical}=413.2804; m/zₑₓₚₑᵣᵢₘₑₙₜₐₗ=413.2789.

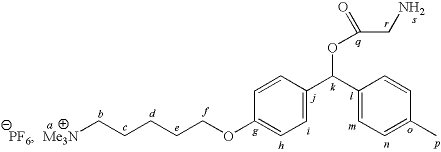

### [Gly-HTMPPTMA][PF₆]

Procedure: cf procedure 9 using Fmoc-Gly.

The yield is 98% over 4 stages.

viscous brown oil

NMR¹H (300 MHz, acetone d₆): δ(Hₐ)=3.33 (s, 9H); δ(H_{b+r})=3.53-3.62 (m, 2H+1H); δ(H_{c})=1.88 (m, 2H); δ(H_{d})=1.59 (m, 2H); δ(Hₑ)=2.05 (m, 2H); δ(H_{f})=4.03 (t, J=6.2, 2H); δ(Hₕ)=6.91 (d, J=8.7, 2H); δ(H₁)=7.18 (d, J=8.0, 2H); δ(Hₘ₊ₙ)=7.29-7.38 (m, 2H+2H); δ(Hₖ)=6.83 (s, 1H); δ(Hₚ)=2.31 (s, 3H).

NMR¹³C (75 MHz, acetone d₆): δ(Cₐ)=52.57; δ(C_{b})=66.35; δ(C_{c})=22.65; δ(C_{d})=22.32; δ(Cₑ)=28.45; δ(C_{f})=67.38; δ(C_{g})=158.80; δ(Cₕ)=114.51; δ(Cᵢ)=128.46; δ(Cⱼ)=132.90; δ(Cₖ)=76.78; δ(Cₗ)=137.41; δ(Cₘ)=126.73; δ(Cₙ)=129.21; δ(Cₒ)=138.11;

δ(Cₚ)=20.45; δ(C_{q})=171.21; δ(Cᵣ)=53.30.

HRMS (ESI) of (C₂₄H₃₅N₂O₃): [C⁺] m/z_{theoretical}=399.2648; m/zₑₓₚₑᵣᵢₘₑₙₜₐₗ=399.2649.

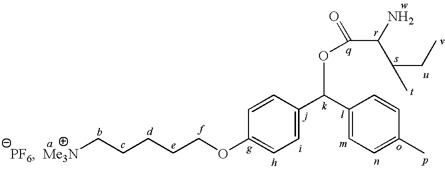

### [Ile-HTMPPTMA][PF₆]

Procedure: cf procedure 9 using Fmoc-Ile.

The yield is 89% over 4 stages.

viscous brown oil

NMR¹H (300 MHz, acetone d₆): δ(Hₐ)=3.33 (s, 9H); δ(H_{b+r})=3.48-3.60 (m, 2H+1H); δ(H_{c+s})=1.79-1.97 (m, 2H+1H); δ(H_{d})=1.59 (m, 2H); δ(Hₑ)=2.05 (m, 2H); δ(H_{f})=4.03 (t, J=6.2, 2H); δ(Hₕ)=6.91 (d, J=8.6, 2H); δ(Hᵢ)=7.18 (d, J=10.3, 2H); δ(Hₖ)=6.80 (s, 1H); δ(Hₘ₊ₙ)=7.19-7.32 (m, 2H+2H); δ(Hₚ)=2.31 (s, 3H); δ(Hₜ₊ᵥ)=0.80-0.96 (m, 3H+3H); δ(Hᵤ)=1.17 (m, 1H); δ(H_{u′})=1.49 (m, 1H).

NMR¹³C (75 MHz, acetone d₆): δ(Cₐ)=52.58; δ(C_{b})=66.36; δ(C_{c})=22.67; δ(C_{d})=22.34; δ(Cₑ)=28.47; δ(C_{f})=67.34; δ(C_{g})=158.77; δ(Cₕ)=114.39; δ(Cᵢ)=128.58; δ(Cⱼ)=132.88; δ(Cₖ)=76.47; δ(Cₗ)=137.33; δ(Cₘ)=126.69; δ(Cₙ)=129.13; δ(Cₒ)=138.08; δ(Cₚ)=20.43; δ(C_{q})=170.65; δ(Cᵣ)=68.96; δ(Cₛ)=38.22; δ(Cₜ)=15.27; δ(Cᵤ)=24.88; δ(Cᵥ)=10.93.

HRMS (ESI) of (C₂₈H₄₃N₂O₃): [C⁺] m/z_{theoretical}=455.3274; m/zₑₓₚₑᵣᵢₘₑₙₜₐₗ=455.3286.

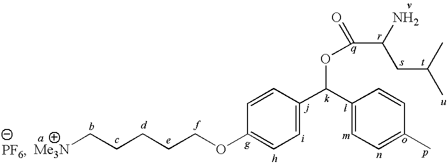

### [Leu-HTMPPTMA][PF₆]

Procedure: cf procedure 9 using Fmoc-Leu.

The yield is 89% over 4 stages.

viscous brown oil

NMR¹H (300 MHz, acetone d₆): δ(Hₐ)=3.36 (s, 9H); δ(H_{b+r})=3.53-3.67 (m, 2H+1H); δ(H_{c+d+s+s′+t})=1.50-1.97 (m, 2H+2H+1H+1H+1H); δ(Hₑ)=2.05 (m, 2H); δ(H_{f})=4.03 (t, J=6.2, 2H); δ(Hₕ)=6.91 (m, 2H); δ(Hᵢ₊ₘ₊ₙ)=7.09-7.34 (m, 2H+2H+2H); δ(Hₖ)=6.78 (s, 1H); δ(Hₚ)=2.31 (s, 3H); δ(Hᵤ)=0.84 (d, J=6.5, 3H); δ(Hₘ)=0.91 (d, J=6.5, 1H).

NMR¹³C (75 MHz, acetone d₆): δ(Cₐ)=52.59; δ(C_{b})=66.36; δ(C_{c})=22.34; δ(C_{d})=22.66; δ(Cₑ)=28.47; δ(C_{f})=67.32; δ(C_{g})=158.75; δ(Cₕ)=114.43; δ(Cᵢ)=128.37; δ(Cⱼ)=132.88; δ(Cₖ)=76.57; δ(Cₗ)=137.28; δ(Cₘ)=126.70; δ(Cₙ)=129.13; δ(Cₒ)=138.16; δ(Cₚ)=20.42; δ(C_{q})=171.27; δ(Cᵣ)=61.98; δ(Cₛ)=42.21; δ(Cₜ)=24.59; δ(Cᵤ)=21.69.

HRMS (ESI) of (C₂₈H₄₃N₂O₃): [C⁺] m/z_{theoretical}=455.3274; m/zₑₓₚₑᵣᵢₘₑₙₜₐₗ=455.3272.

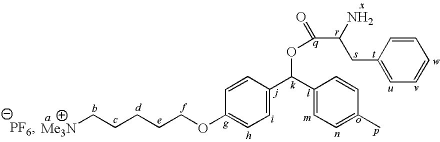

### [Phe-HTMPPTMA][PF₆]

Procedure: cf procedure 9 using Fmoc-Phe.

The yield is 80% over 4 stages.

viscous brown oil

NMR¹H (300 MHz, acetone d₆): δ(Hₐ)=3.33 (s, 9H); δ(H_{b+r})=3.50-3.61 (m, 2H+1H); δ(H_{c})=1.93 (m, 2H); δ(H_{d})=1.60 (m, 2H); δ(Hₑ)=2.05 (m, 2H); δ(H_{f})=4.03 (t, J=6.1, 2H); δ(Hₕ)=6.88 (m, 2H); δ(H_{i+m+n+u+v+w})=7.09-7.33 (m, 2H+2H+2H+2H+2H+1H); δ(Hₖ)=6.77 (s, 1H); δ(Hₚ)=2.31 (s, 3H); δ(H_{s+s′})=2.87-3.06 (m, 1H+1H).

NMR¹³C (75 MHz, acetone d₆): δ(Cₐ)=52.61; δ(C_{b})=66.38; δ(C_{c})=22.69; δ(C_{d})=22.35; δ(Cₑ)=28.49; δ(C_{f})=67.35; δ(C_{g})=158.75; δ(Cₕ)=114.44; δ(Cᵢ)=129.13; δ(Cⱼ)=132.75; δ(Cₖ)=76.79; δ(Cₗ)=137.34; δ(Cₘ)=126.72; δ(Cₙ)=129.62; δ(Cₒ)=138.27; δ(Cₚ)=20.44; δ(C_{q})=170.67; δ(Cᵣ)=65.45; δ(Cₛ)=39.29; δ(Cₜ)=138.03; δ(Cᵤ)=128.47; δ(Cᵥ)=128.28; δ(C_{w})=126.42.

HRMS (ESI) of (C₃₁H₄₁N₂O₃): [C⁺] m/z_{theoretical}=489.3117; m/zₑₓₚₑᵣᵢₘₑₙₜₐₗ=489.3121.

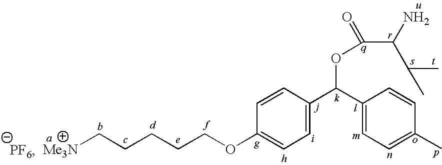

### [Val-HTMPPTMA][PF₆]

Procedure: cf procedure 9 using Fmoc-Val.

The yield is 80% over 4 stages.

viscous brown oil

NMR¹H (300 MHz, acetone d₆): δ(Hₐ)=3.34 (s, 9H); δ(H_{b+r})=3.50-3.61 (m, 2H+1H); δ(H_{c})=1.87 (m, 2H); δ(H_{d})=1.60 (m, 2H); δ(Hₑ)=2.05 (m, 2H); δ(H_{f})=4.03 (t, J=6.1, 2H); δ(Hₕ)=6.90 (d, J=8.7, 2H); δ(Hᵢ₊ₘ₊ₙ)=7.12-7.36 (m, 2H+2H+2H); δ(Hₖ)=6.79 (s, 1H); δ(Hₚ₊ₛ)=2.23-2.39 (m, 3H+1H); δ(H_{t+c})=0.80-1.00 (m, 3H+3H).

NMR¹³C (75 MHz, acetone d₆): δ(Cₐ)=52.59; δ(C_{b})=66.37; δ(C_{c})=22.66; δ(C_{d})=22.34; δ(Cₑ)=28.46; δ(C_{f})=67.33; δ(C_{g})=158.76; δ(Cₕ)=114.40; δ(Cᵢ)=128.33; δ(Cⱼ)=132.91; δ(Cₖ)=76.45; δ(Cₗ)=137.34; δ(Cₘ)=126.66; δ(Cₙ)=129.11; δ(Cₒ)=138.10; δ(Cₚ)=20.41; δ(C_{q})=170.65; δ(Cᵣ)=69.79; δ(Cₛ)=31.70; δ(Cₜ)=18.01; δ(C_{t′})=18.94.

HRMS (ESI) of (C₂₇H₄₁N₂O₃): [C⁺] m/z_{theoretical}=441.3117; m/zₑₓₚₑᵣᵢₘₑₙₜₐₗ=441.3120.

2.3.2. Synthesis of Supported Dipeptides.

General procedure 10 for direct route peptide coupling with onium trifluoromethane sulphonate supports:

1.0 eq. of the supported peptide to be coupled is dissolved in acetonitrile, then 1.5 eq. of DCC, HOBT, TEA and Fmoc-amino acid are added. The medium is stirred for 2 hours at AT then the mixture is filtered. The acetonitrile is evaporated off and the residue obtained is washed with ether then dissolved in dichloromethane. This phase is washed with three times one-tenth by volume of a 1N aqueous solution of HCl before being dried over sodium sulphate and filtered. The DCM is evaporated off.

With the support [HHeTMA]

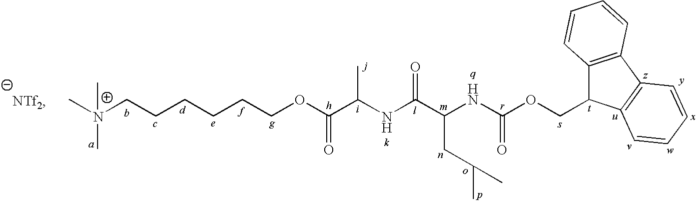

### [Fmoc-Leu-Ala-HHeTMA][NTf₂]

Procedure: cf general procedure 10 using [Ala-HHeTMA][NTf₂] and Fmoc-leucine.

The yield is 78%.

viscous yellow oil

NMR¹H (300 MHz, acetone d⁶): δ(Hₐ)=3.37 (s, 9H); δ(H_{b})=3.58 (m, 2H); δ(H_{c+n})=1.59-1.70 (m, 2H+2H); δ(H_{d+e})=1.40-1.53 (m, 2H+2H); δ(H_{f})=1.97 (m, 2H); δ(H_{g})=4.10 (t, J=6.4, 2H); δ(Hᵢ₊ₘ₊ₛ₊ₜ)=4.19-4.46 (m, 1H+1H+2H+1H); δ(Hⱼ)=1.36 (d, J=7.3, 3H); δ(Hₖ)=7.59 (m, 1H); δ(Hₒ)=1.78 (seven, 1H); δ(Hₚ)=0.93 (d, J=6.6, 3H); δ(H_{p′})=0.95 (d, J=6.6, 3H); δ(H_{q})=6.62 (m, 1H); δ(Hᵥ)=7.71 (d, J=7.4, 2H); δ(H_{w+x})=7.32-7.46 (m, 2H+2H); δ(H_{y})=7.89 (d, J=7.5, 2H).

NMR¹³C (75 MHz, acetone d⁶): δ(Cₐ)=52.71 (t, J_{N-C}=4.0); δ(C_{b})=66.45; δ(C_{c})=22.44; δ(C_{d})=25.49; δ(Cₑ)=25.00; δ(C_{f})=28.07; δ(C_{g})=64.48; δ(Cₕ)=172.44; =48.16; δ(Cⱼ)=16.92; δ(Cₗ)=172.59; δ(Cₘ)=53.41; δ(Cₙ)=41.43; δ(Cₒ)=24.50; δ(Cₚ)=21.19; δ(Cᵣ)=156.28; δ(Cₛ)=66.45; δ(Cₜ)=47.14; δ(Cᵤ)=144.17; δ(Cᵥ)=125.29; δ(C_{w})=127.15; δ(Cₓ)=127.77; δ(C_{y})=120.02; δ(C_{z})=141.20; δ(C_{NTf2})=120.04 (q, J_{C-F}=321.4).

HRMS (LSIMS) of (C₃₃H₄₈N₃O₅): [M⁺] m/z_{theoretical}=566.35940; m/zₑₓₚₑᵣᵢₘₑₙₜₐₗ=566.3603.

With the Support [HMPhTMA]

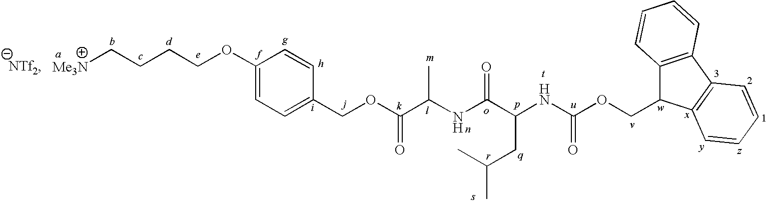

### [Fmoc-Leu-Ala-HMPhBTMA][NTf₂]

Procedure: cf general procedure 10 using [Ala-HMPhBTMA][NTf₂] and Fmoc-leucine. The yield is 85%.

viscous yellow oil

NMR¹H (300 MHz, acetone d₆): δ(Hₐ)=3.40 (s, 9H); δ(H_{b})=3.69 (m, 2H); δ(H_{c})=1.91 (m, 2H); δ(H_{d})=2.16 (m, 2H); δ(Hₑ)=4.09 (t, J=5.9, 2H); δ(H_{g})=6.92 (d, J=8.5, 2H); δ(H_{h+z})=7.28-7.37 (m, 2H+2H); δ(Hⱼ)=5.08 (s, 2H); δ(H_{l+v+w})=4.17-4.39 (m, 1H+2H+1H); δ(Hₘ)=1.36 (d, J=7.3, 3H); δ(Hₙ)=6.62 (m, 1H); δ(Hₚ)=4.47 (m, 1H); δ(H_{q+q′})=1.60 (m, 1H+1H); δ(Hᵣ)=1.76 (m, 1H); δ(Hₛ)=0.91 (d, J=6.9, 3H); δ(H_{s′})=0.93 (d, J=7.0, 3H); δ(Hₜ)=7.61 (m, 1H); δ(H_{y})=7.72 (m, 2H); δ(H₁)=7.43 (m, 2H); δ(H₂)=7.88 (d, J=7.5, 2H).

NMR¹³C (100 MHz, acetone d₆): δ(Cₐ)=54.13; δ(C_{b})=67.62; δ(C_{c})=25.93; δ(C_{d})=27.14; δ(Cₑ)=68.06; δ(C_{f})=157.39; δ(C_{g})=115.64; δ(Cₕ)=131.20; δ(Cᵢ)=129.68; δ(Cⱼ)=67.35; δ(Cₖ)=173.56; δ(Cₗ)=49.32; δ(Cₘ)=18.14; δ(Cₒ)=173.56; δ(Cₚ)=54.48; δ(C_{q})=42.70; δ(Cᵣ)=23.93; δ(C_{S})=21.15; δ(C_{s′})=22.35; δ(Cᵤ)=160.17; δ(Cᵥ)=67.50; δ(C_{w})=48.43; δ(Cₓ)=145.52; δ(C_{y})=126.57; δ(C_{z})=128.34; δ(C₁)=128.96; δ(C₂)=121.23; δ(C₃)=142.49; δ(C_{NTf2})=121.43 (q, J_{C-F}=321.2).

HRMS (ESI) of (C₃₈H₅₀N₃O₆): [C⁺] m/z_{theoretical}=644.3700; m/zₑₓₚₑᵣᵢₘₑₙₜₐₗ=644.3699.

With the support [HTMPPTMA][PF₆]

General procedure 11 for direct route peptide coupling with onium hexafluorophosphate supports

1.0 eq. of supported peptide having the deprotected amine is dissolved in acetonitrile then 1.5 eq. of TEA, Fmoc-amino acid and
either 1.5 eq. of HOBt and carbodiimide (DCC or DIC) are added.
or 1.5 eq. of HBTU

The reaction medium is stirred for 30 minutes at AT.

If the coupling reagents are DCC/HOBt, the reaction medium is filtered (DCU poorly soluble in acetonitrile) then the acetonitrile is evaporated off. Otherwise the acetonitrile is evaporated directly.

The residue obtained is then washed with ether then it is dissolved in dichloromethane. This phase is washed three times with water then three times with an aqueous solution of HPF₆ (1<pH<2) before being dried over sodium sulphate then filtered. The dichloromethane is evaporated off.

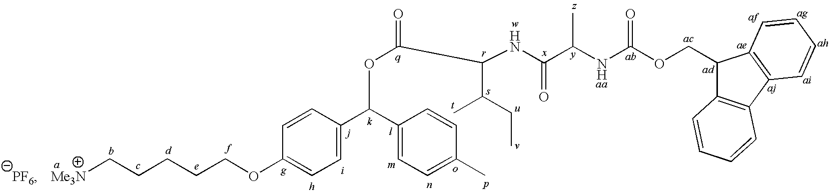

### [Fmoc-Ala-Ile-HTMPPTMA][PF₆]

Procedure: cf general procedure 11 using [Ile-HTMPPTMA][PF₆] and Fmoc-alanine.

The yield is 88%.

viscous yellow oil

NMR¹H (300 MHz, acetone d₆): δ(Hₐ)=3.37 (s, 9H); δ(H_{b})=3.60 (m, 2H); δ(H_{c})=1.86 (m, 2H); δ(H_{d})=1.59 (m, 2H); δ(Hₑ₊ₛ)=1.97-2.09 (m, 2H+1H); δ(H_{f})=4.00 (m, 2H); δ(Hₕ₊ₖ₊ₐₐ)=6.68-6.94 (m, 2H+1H+1H); δ(H_{i+m+n+w+ag+ah})=7.03-7.50 (m, 2H+2H+2H+1H+2H+2H); δ(Hₚ)=2.23-2.33 (m, 3H); δ(Hᵣ)=4.53 (m, 1H); δ(Hₜ₊ᵥ)=0.78-0.90 (m, 3H+3H); δ(H_{u+u′})=1.03-1.28 (m, 1H+1H); δ(H_{y+ac+ad})=4.21-4.40 (m, 1H+2H+1H); δ(H_{z})=1.35 (d, J=7.0, 3H); δ(H_{af})=7.71 (m, 2H); δ(Hₐᵢ)=7.88 (d, J=7.5, 2H).

NMR¹³C (75 MHz, acetone d₆): δ(Cₐ)=53.56; δ(C_{b})=67.44; δ(C_{c})=23.58; δ(C_{d})=23.28; δ(Cₑ)=29.65; δ(C_{f})=68.20; δ(C_{g})=159.74; δ(Cₕ)=115.25; δ(Cᵢ)=129.56; δ(Cⱼ)=133.41; δ(Cₖ)=78.16; δ(Cₗ)=138.24; δ(Cₘ)=127.56; δ(Cₙ)=129.97; δ(Cₒ)=138.59; δ(Cₚ)=21.32; δ(C_{q})=171.40; δ(Cᵣ)=57.80; δ(Cₛ)=38.16; δ(Cₜ)=16.13; δ(Cᵤ)=25.69; δ(Cᵥ)=11.88; δ(Cₓ)=173.76; δ(C_{y})=51.34; δ(C_{z})=18.83; δ(C_{ab})=156.98; δ(C_{ac})=67.29; δ(C_{ad})=47.99; δ(Cₐₑ)=145.04; δ(C_{af})=126.23; δ(C_{ag})=128.11; δ(Cₐₕ)=128.70; δ(Cₐᵢ)=120.93; δ(Cₐⱼ)=142.10.

HRMS (ESI) of (C₄₆H₅₈N₁₃O₆): [C⁺] m/z_{theoretical}=748.4325; m/zₑₓₚₑᵣᵢₘₑₙₜₐₗ=748.4319.

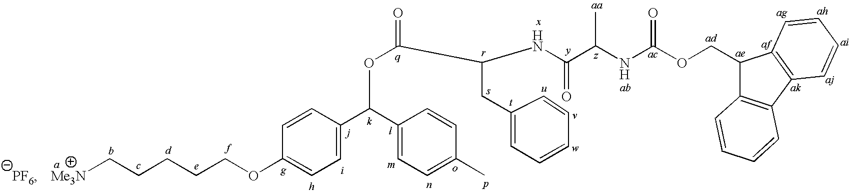

### [Fmoc-Ala-Phe-HTMPPTMA][PF₆]

Procedure: cf general procedure 11 using [Phe-HTMPPTMA][PF₆] and Fmoc-alanine. The yield is 98%.

viscous yellow oil

NMR¹H (300 MHz, acetone d₆): δ(Hₐ)=3.36 (s, 9H); δ(H_{b})=3.59 (m, 2H); δ(H_{c})=1.83 (m, 2H); δ(H_{d})=1.61 (m, 2H); δ(Hₑ)=2.04 (m, 2H); δ(H_{f})=4.01 (m, 2H); δ(Hₕ)=6.88 (m, 2H); δ(H_{i+m+n+u+v+w+ah+ai})=7.05-7.48 (m, 2H+2H+2H+2H+2H+1H+2H+2H); δ(Hₖ)=6.79 (s, 1H); δ(Hₚ)=2.30 (d, J=10.0, 3H); δ(Hᵣ)=4.86 (m, 1H); δ(H_{s+s′})=2.92-3.25 (m, 1H+1H); δ(Hₓ)=7.51 (m, 1H); δ(H_{z+ad+ae})=4.12-4.36 (m, 1H+2H+1H); δ(Hₐₐ)=1.30 (d, J=7.1, 3H); δ(H_{ab})=6.70 (m, 1H); δ(H_{ag})=7.71 (m, 2H); δ(Hₐⱼ)=7.88 (d, J=7.4, 2H).

NMR¹³C (75 MHz, acetone d₆): δ(Cₐ)=53.51; δ(C_{b})=66.48; δ(C_{c})=23.60; δ(C_{d})=22.27; δ(Cₑ)=29.67; δ(C_{f})=68.24; δ(C_{g})=159.67; δ(Cₕ)=115.35; δ(Cᵢ)=130.07; δ(Cⱼ)=133.36; δ(Cₖ)=78.46; δ(Cₗ)=137.59; δ(Cₘ)=127.60; δ(Cₙ)=130.30; δ(Cₒ)=138.54; δ(Cₚ)=21.34; δ(C_{q})=171.24; δ(Cᵣ)=54.78; δ(Cₛ)=38.07; δ(Cₜ)=138.24; δ(Cᵤ)=129.63; δ(Cᵥ)=129.33; δ(C_{w})=127.46; δ(C_{y})=173.53; δ(C_{z})=51.43; δ(Cₐₐ)=18.81; δ(C_{ac})=156.96; δ(C_{ad})=67.26; δ(Cₐₑ)=48.00; δ(C_{af})=145.06; δ(C_{ag})=126.29; δ(Cₐₕ)=127.69; δ(Cₐᵢ)=127.90; δ(Cₐⱼ)=121.00; δ(Cₐₖ)=142.13.

HRMS (ESI) of (C₄₉H₅₆N₃O₆): [C⁺] m/z_{theoretical}=782.4169; m/zₑₓₚₑᵣᵢₘₑₙₜₐₗ=782.4175.

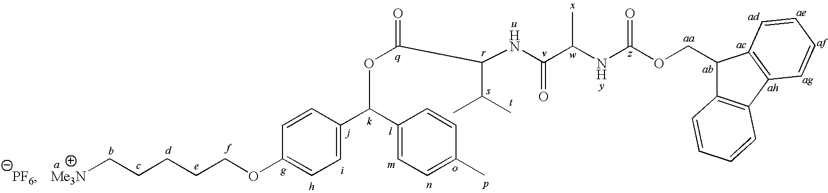

### [Fmoc-Ala-Val-HTMPPTMA][PF₆]

Procedure: cf general procedure 11 using [Val-HTMPPTMA][PF₆] and Fmoc-alanine.

The yield is 70%.

viscous yellow oil

NMR¹H (300 MHz, acetone d₆): δ(Hₐ)=3.36 (s, 9H); δ(H_{b})=3.59 (m, 2H); δ(H_{c})=1.83 (m, 2H); δ(H_{d})=1.57 (m, 2H); δ(Hₑ)=2.04 (m, 2H); δ(H_{f})=4.00 (m, 2H); δ(Hₕ)=6.88 (m, 2H); δ(H_{i+m+n+u+ae+af})=7.06-7.49 (m, 2H+2H+2H+1H+2H+2H); δ(Hₖ)=6.82 (s, 1H); δ(Hₚ)=2.30 (d, J=4.7, 3H); δ(Hᵣ)=4.52 (m, 1H); δ(Hₛ)=2.22 (m, 1H); δ(Hₜ)=0.86 (d, J=6.9, 3H); δ(H_{t′})=0.90 (d, J=6.8, 3H); δ(H_{w+aa+ab})=4.16-4.40 (m, 1H+2H+1H); δ(Hₓ)=1.35 (d, J=7.1, 3H); δ(H_{y})=6.72 (m, 1H); δ(H_{ad})=7.71 (m, 2H); δ(H_{ag})=7.88 (d, J=7.4, 2H).

NMR¹³C (75 MHz, acetone d₆): δ(Cₐ)=53.53; δ(C_{b})=67.29; δ(C_{c})=23.58; δ(C_{d})=23.26; δ(Cₑ)=29.58; δ(C_{f})=68.16; δ(C_{g})=159.73; δ(Cₕ)=115.24; δ(Cᵢ)=129.52; δ(Cⱼ)=133.44; δ(Cₖ)=78.13; δ(Cₗ)=138.22; δ(Cₘ)=127.80; δ(Cₙ)=129.97; δ(Cₒ)=138.63; δ(Cₚ)=21.26; δ(C_{q})=171.38; δ(Cᵣ)=58.50; δ(Cₛ)=31.62; δ(Cₜ)=18.28; δ(C_{t′})=19.66; δ(Cᵥ)=173.74; δ(C_{w})=51.31; δ(Cₓ)=18.80; δ(C_{z})=156.93; δ(Cₐₐ)=67.29; δ(C_{ab})=47.99; δ(C_{ac})=145.05; δ(C_{ad})=126.22; δ(Cₐₑ)=128.10; δ(C_{af})=128.69; δ(C_{ag})=120.93; δ(Cₐₕ)=142.10.

HRMS (ESI) of (C₄₅H₅₆N₃O₆): [C⁺] m/z_{theoretical}=734.4169; m/zₑₓₚₑᵣᵢₘₑₙₜₐₗ=734.4173.

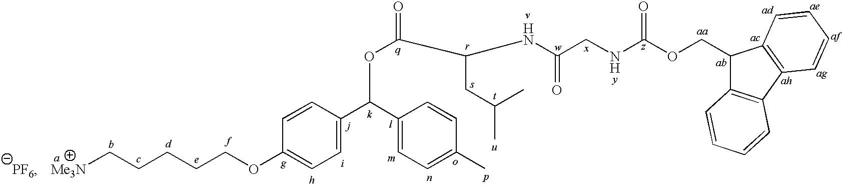

### [Fmoc-Gly-Leu-HTMPPTMA][PF₆]

Procedure: cf general procedure 11 using [Leu-HTMPPTMA][PF₆] and Fmoc-glycine.

The yield is 70%.

viscous yellow oil

NMR¹H (300 MHz, acetone d₆): δ(Hₐ)=3.35 (s, 9H); δ(H_{b})=3.59 (m, 2H); δ(H_{c})=1.85 (m, 2H); δ(H_{d+s+s′+t})=1.48-1.75 (m, 2H+1H+1H+1H); δ(Hₑ)=2.05 (m, 2H); δ(H_{f+x})=3.81-4.08 (m, 2H+2H); δ(H_{h+y})=6.80-6.95 (m, 2H+1H); δ(H_{i+m+n+v+ae+af})=7.01-7.57 (m, 2H+2H+2H+1H+2H+2H); δ(Hₖ)=6.78 (s, 1H); δ(Hₚ)=2.29 (m, 3H); δ(Hᵣ)=4.62 (m, 1H); δ(Hᵤ)=0.88 (d, J=4.0, 6H); δ(H_{aa+ab})=4.20-4.42 (m, 2H+1H); δ(H_{ad})=7.73 (m, 2H); δ(H_{ag})=7.88 (d, J=7.5, 2H).

NMR¹³C (75 MHz, acetone d₆): δ(Cₐ)=53.54; δ(C_{b})=67.56; δ(Cᵣ)=23.57; δ(C_{d})=23.26; δ(Cₑ)=29.62; δ(C_{f})=68.16; δ(C_{g})=159.67; δ(Cₕ)=115.30; δ(Cᵢ)=129.28; δ(Cⱼ)=133.53; δ(Cₖ)=78.14; δ(Cₗ)=138.23; δ(Cₘ)=127.62; δ(Cₙ)=130.03; δ(Cₒ)=138.71; δ(Cₚ)=21.29; δ(C_{q})=170.45; δ(Cᵣ)=51.94; δ(Cₛ)=41.24; δ(Cᵣ)=25.47; δ(Cₙ)=22.12; δ(C_{w})=172.36; δ(C_{w})=44.74; δ(C_{z})=157.67; δ(Cₐₐ)=67.29; δ(C_{ab})=47.97; δ(C_{ac})=145.00; δ(C_{ad})=126.25; δ(Cₐₑ)=128.12; δ(C_{af})=128.73; δ(C_{ag})=120.96; δ(Cₐₕ)=142.10.

HRMS (ESI) of (C₄₅H₅₆N₃O₆): [C⁺] m/z_{theoretical}=734.4169; m/zₑₓₚₑᵣᵢₘₑₙₜₐₗ=734.4170.

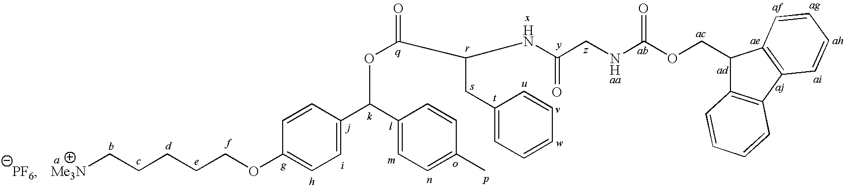

### [Fmoc-Gly-Phe-HTMPPTMA][PF₆]

Procedure: cf general procedure 11 using [Phe-HTMPPTMA][PF₆] and Fmoc-glycine. The yield is 85%.

viscous yellow oil

NMR¹H (300 MHz, acetone d₆): δ(Hₐ)=3.36 (s, 9H); δ(H_{b})=3.59 (m, 2H); δ(H_{c})=1.85 (m, 2H); δ(H_{d})=1.61 (m, 2H); δ(Hₑ)=2.04 (m, 2H); δ(H_{f})=4.02 (m, 2H); δ(H_{b})=6.88 (m, 2H); δ(H_{i+m+n+u+v+w+aa+ag+ah})=7.05-7.45 (m, 2H+2H+2H+2H+2H+1H+1H+2H+2H); δ(Hₖ)=6.78 (s, 1H); δ(Hₚ)=2.30 (d, J=4.4, 3H); δ(Hᵣ)=4.86 (m, 1H); δ(H_{s+s′})=2.96-3.24 (m, 1H+1H); δ(Hₓ)=7.54 (m, 1H); δ(H_{z})=3.86 (m, 2H); δ(H_{ac+ad})=4.17-4.40 (m, 2H+1H); δ(H_{af})=7.73 (d, J=7.2, 2H); δ(Hₐᵢ)=7.88 (d, J=7.4, 2H).

NMR¹³C (75 MHz, acetone d₆): δ(Cₐ)=53.53; δ(C_{b})=67.52; δ(C_{c})=23.58; δ(C_{d})=23.25; δ(Cₑ)=29.35; δ(C_{f})=68.15; δ(C_{g})=159.72; δ(Cₕ)=115.25; δ(Cᵢ)=129.85; δ(Cⱼ)=133.40; δ(Cₖ)=78.35; δ(Cₗ)=137.55; δ(Cₘ)=127.83; δ(Cₙ)=130.00; δ(Cₒ)=138.76; δ(Cₚ)=21.24; δ(C_{q})=170.12; δ(Cᵣ)=54.67; δ(Cₛ)=38.09; δ(Cₜ)=138.19; δ(Cᵤ)=129.30; δ(Cᵥ)=129.24; δ(C_{w})=127.58; δ(C_{y})=171.19; δ(C_{z})=44.74; δ(C_{ab})=157.61; δ(C_{ac})=67.28; δ(C_{ad})=47.96; δ(Cₐₑ)=145.02; δ(C_{af})=126.23; δ(C_{ag})=128.10; δ(Cₐₕ)=128.69; δ(Cₐᵢ)=120.93; δ(Cₐⱼ)=142.11.

HRMS (ESI) of (C₄₈H₅₄N₃O₆): [C⁺] m/z_{theoretical}=768.4012; m/zₑₓₚₑᵣᵢₘₑₙₜₐₗ=768.4012.

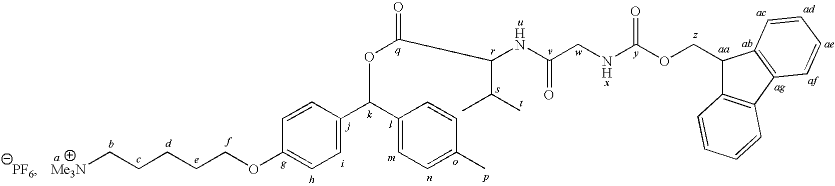

### [Fmoc-Gly-Val-HTMPPTMA][PF₆]

Procedure: cf general procedure 11 using [Val-HTMPPTMA][PF₆] and Fmoc-glycine.

The yield is 95%.

viscous yellow oil

NMR¹H (300 MHz, acetone d₆): δ(Hₐ)=3.35 (s, 9H); δ(H_{b})=3.58 (m, 2H); δ(H_{c})=1.85 (m, 2H); δ(H_{d})=1.58 (m, 2H); δ(Hₑ)=2.04 (m, 2H); δ(H_{f+w})=3.88-4.08 (m, 2H+2H); δ(Hₕ₊ₓ)=6.84-6.98 (m, 2H+1H); δ(H_{i+m+n+u+ad+ae})=7.09-7.51 (m, 2H+2H+2H+1H+2H+2H); δ(Hₖ)=6.82 (s, 1H); δ(Hₚ)=2.30 (s, 3H); δ(Hᵣ)=4.56 (m, 1H); δ(Hₛ)=2.22 (m, 1H); δ(Hₜ)=0.86 (d, J=7.0, 3H); δ(H_{t′})=0.90 (d, J=6.8, 3H); δ(H_{z+aa})=4.20-4.40 (m, 2H+1H); δ(H_{ac})=7.73 (m, 2H); δ(H_{ag})=7.88 (d, J=7.5, 2H).

NMR¹³C (75 MHz, acetone d₆): δ(Cₐ)=53.52; δ(C_{b})=67.60; δ(C_{c})=23.57; δ(C_{d})=23.26; δ(Cₑ)=29.66; δ(C_{f})=68.18; δ(C_{g})=159.66; δ(Cₕ)=115.29; δ(Cᵢ)=129.49; δ(Cⱼ)=133.43; δ(Cₖ)=78.22; δ(Cₗ)=138.27; δ(Cₘ)=127.60; δ(Cₙ)=130.03; δ(Cₒ)=138.57; δ(Cₚ)=21.33; δ(C_{q})=170.60; δ(Cᵣ)=58.58; δ(Cₛ)=31.65; δ(Cₜ)=18.35; δ(C_{t′})=19.71; δ(Cᵥ)=171.52; δ(C_{w})=44.80; δ(C_{y})=157.76; δ(C_{z})=67.27; δ(Cₐₐ)=47.96; δ(C_{ab})=144.99; δ(C_{ac})=126.25; δ(C_{ad})=128.15; δ(Cₐₑ)=128.75; δ(C_{af})=120.97; δ(C_{ag})=142.10.

HRMS (ESI) of (C₄₄H₅₄N₃O₆): m/z_{theoretical}=720.4013; m/zₑₓₚₑᵣᵢₘₑₙₜₐₗ=720.4015.

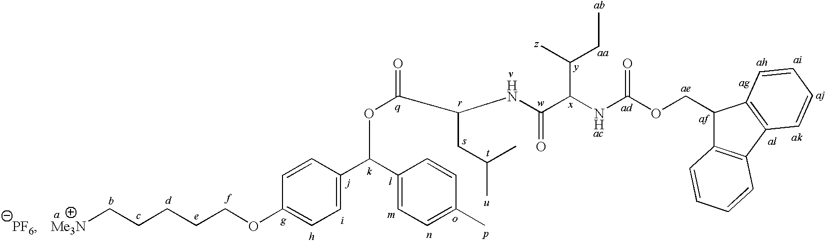

### [Fmoc-Ile-Leu-HTMPPTMA][PF₆]

Procedure: cf general procedure 11 using [Leu-HTMPPTMA][PF₆] and Fmoc-isoleucine. The yield is 78%.

viscous yellow oil

NMR¹H (300 MHz, acetone d₆): δ(Hₐ)=3.37 (s, 9H); δ(H_{b})=3.58 (m, 2H); δ(H_{c})=1.86 (m, 2H); δ(H_{d+s+s′+t+aa})=1.45-1.75 (m, 2H+1H+1H+1H+1H); δ(H_{e+y})=1.94-2.12 (m, 2H+1H); δ(H_{f+x})=3.94-4.17 (m, 2H+1H); δ(Hₕ)=6.91 (m, 2H); δ(Hᵢ₊ₘ₊ₙ₊ₐᵢ₊ₐⱼ)=7.03-7.50 (m, 2H+2H+2H+2H+2H); δ(Hₖ)=6.80 (s, 1H); δ(Hₚ)=2.31 (m, 3H); δ(Hᵣ)=4.65 (m, 1H); δ(H_{u+u′+z+ab})=0.73-0.98 (m, 3H+3H+3H+3H); δ(Hᵥ)=7.48 (m, 1H); δ(H_{aa′})=1.17 (m, 1H); δ(H_{ac})=6.55 (m, 1H); δ(H_{ae+af})=4.19-4.40 (m, 2H+1H); δ(H_{ab})=7.72 (m, 2H); δ(Hₐₖ)=7.88 (d, J=7.6, 2H).

NMR¹³C (75 MHz, acetone d₆): δ(Cₐ)=53.49; δ(C_{b})=67.27; δ(Cᵣ)=23.34; δ(C_{d})=23.26; δ(Cₑ)=29.68; δ(C_{f})=68.22; δ(C_{g})=159.74; δ(Cₕ)=115.32; δ(Cᵢ)=129.51; δ(Cⱼ)=133.46; δ(Cₖ)=78.21; δ(Cₗ)=138.19; δ(Cₘ)=127.81; δ(Cₙ)=130.05; δ(Cₒ)=138.67;

δ(Cₚ)=21.36; δ(C_{q})=172.39; δ(Cᵣ)=51.92; δ(Cₛ)=41.26; δ(Cₜ)=25.60; δ(Cᵤ)=22.14; δ(C_{w})=172.62; δ(Cₓ)=60.47; δ(C_{y})=38.15; δ(C_{z})=16.19; δ(Cₐₐ)=25.46; δ(C_{ab})=11.72; δ(C_{ad})=157.23; δ(Cₐₑ)=67.27; δ(C_{af})=48.07; δ(C_{ag})=145.10; δ(Cₐₕ)=126.25; δ(Cₐᵢ)=128.13; δ(Cₐⱼ)=128.74; δ(Cₐₖ)=120.98; δ(Cₐₗ)=142.11.

HRMS (ESI) of (C₄₉H₆₄N₃O₆): [C⁺] m/z_{theoretical}=790.4795; m/zₑₓₚₑᵣᵢₘₑₙₜₐₗ=790.4798.

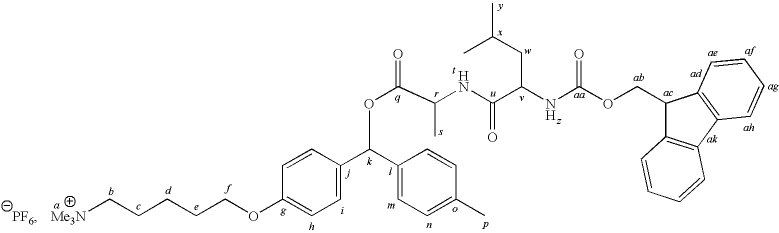

### [Fmoc-Leu-Ala-HTMPPTMA][PF₆]

Procedure: cf general procedure 11 using [Ala-HTMPPTMA][PF₆] and Fmoc-leucine.

The yield is 94%.

viscous yellow oil

NMR¹H (300 MHz, acetone d₆): δ(Hₐ)=3.32 (s, 9H); δ(H_{b})=3.56 (m, 2H); δ(H_{c+d+w)=}1.50-1.67 (m, 2H+2H+2H); δ(Hₑ)=1.85 (m, 2H); δ(H_{f})=4.00 (m, 2H); δ(Hₕ)=6.89 (dd, J₁=8.5, J₂=3.4, 2H); δ(H_{i+m+n+af+ag})=7.11-7.50 (m, 2H+2H+2H+2H+2H); δ(Hₖ)=6.78 (s, 1H); δ(Hₚ)=2.30 (d, J=4.5, 3H); δ(H_{r+ab+ac})=4.18-4.43 (m, 1H+2H+1H); δ(Hₛ)=1.40 (dd, J₁=7.2, J₂=3.2, 3H); δ(Hₜ₊ₐₑ)=7.63-7.74 (m, 1H+2H); δ(Hᵥ)=4.59 (m, 1H); δ(Hₓ)=1.74 (m, 1H); δ(H_{y})=0.90 (d, J=5.8, 3H); δ(H_{y′})=0.92 (d, J=6.1, 3H); δ(H_{z})=6.65 (m, 1H); δ(Hₐₕ)=7.86 (d, J=8.0, 2H).

NMR¹³C (75 MHz, acetone d₆): δ(Cₐ)=52.69 (t, J_{C-N}=4.0); δ(C_{b})=66.43; δ(C_{c})=22.72; δ(C_{d})=22.36; δ(Cₑ)=28.45; δ(C_{f})=67.21; δ(C_{g})=158.74; δ(Cₕ)=114.31; δ(Cᵢ)=128.34; δ(Cⱼ)=132.75; δ(Cₖ)=77.09; δ(Cₗ)=137.24; δ(Cₘ)=126.69; δ(Cₙ)=129.03; δ(Cₒ)=137.93; δ(Cₚ)=21.03; δ(C_{q})=171.31; δ(Cᵣ)=48.23; δ(Cₛ)=16.83; δ(Cᵤ)=171.41; δ(Cᵥ)=53.30; δ(C_{w})=41.46; δ(Cₓ)=24.47; δ(C_{y})=20.24; δ(Cₐₐ)=156.20; δ(C_{ab})=66.27; δ(C_{ac})=47.16; δ(C_{ad})=144.23; δ(Cₐₑ)=125.29; δ(C_{af})=127.09; δ(C_{ag})=127.70; δ(Cₐₕ)=119.97; δ(Cₐₖ)=141.21.

HRMS (ESI) of (C₄₆H₅₈N₃O₆): [C⁺] m/z_{theoretical}=748.4325; m/zₑₓₚₑᵣᵢₘₑₙₜₐₗ=748.4321.

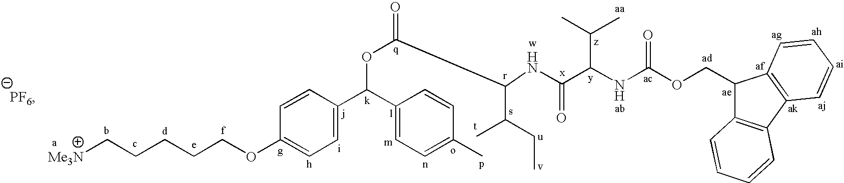

[Fmoc-Val-Ile-HTMPPTMA][PF₆]

Procedure: cf general procedure 11 using [Ile-HTMPPTMA][PF₆] and Fmoc-valine.

The yield is 94%.

viscous yellow oil

NMR¹H (300 MHz, acetone d₆): δ(Hₐ)=3.37 (s, 9H); δ(H_{b})=3.61 (m, 2H); δ(H_{c})=1.86 (m, 2H); δ(H_{d})=1.58 (m, 2H); δ(H_{e+s+z})=1.90-2.09 (m, 2H+1H+1H); δ(H_{f})=4.02 (m, 2H); δ(Hₕ₊ₖ)=6.76-6.94 (m, 2H+1H); δ(H_{i+m+n+w+ah+ai})=7.02-7.52 (m, 2H+2H+2H+1H+2H+2H); δ(Hₚ)=2.30 (d, J=5.1, 3H); δ(Hᵣ)=4.58 (m, 1H); δ(H_{t+v+aa+aa′})=0.77-0.97 (m, 3H+3H+3H+3H); δ(Hᵤ)=1.19 (m, 1H); δ(H_{u′})=1.37 (m, 1H); δ(H_{y+ad+ae})=4.08-4.37 (m, 1H+2H+1H); δ(H_{ab})=6.54 (m, 1H); δ(H_{ag})=7.72 (m, 2H); δ(Hₘ)=7.88 (d, J=7.5, 2H).

NMR¹³C (75 MHz, acetone d₆): δ(Cₐ)=53.53; δ(C_{b})=67.29; δ(C_{c})=23.58; δ(C_{d})=23.27; δ(Cₑ)=29.64; δ(C_{f})=68.21; δ(C_{g})=159.77; δ(Cₕ)=115.24; δ(Cᵢ)=129.69; δ(Cⱼ)=133.40; δ(Cₖ)=78.20; δ(Cₗ)=138.19; δ(Cₘ)=127.53; δ(Cₙ)=129.96; δ(Cₒ)=138.70; δ(Cₚ)=21.33; δ(C_{q})=171.49; δ(Cᵣ)=57.77; δ(Cₛ)=38.16; δ(Cₜ)=16.13; δ(Cᵤ)=25.72; δ(Cᵥ)=11.87; δ(Cₓ)=172.61; δ(C_{y})=61.13; δ(C_{z})=31.90; δ(Cₐₐ)=18.54; δ(C_{aa′})=20.01; δ(Cₐᵣ)=157.35; δ(C_{ad})=67.29; δ(Cₐₑ)=48.05; δ(C_{af})=145.07; δ(C_{ag})=126.22; δ(C_{ab})=128.09; δ(Cₘ)=128.69; δ(Cₘ)=120.93; δ(Cₐₖ)=142.11.

HRMS (ESI) of (C₄₈H₆₂N₃O₆): [C⁺] m/z_{theoretical}=776.4638; m/zₑₓₚₑᵣᵢₘₑₙₜₐₗ=776.4633.

2.3.3. Synthesis of Protected Supported Tripeptides.

General procedure 8′ for the cleavage of the Fmoc group with the support [HTMPPTMA][PF₆]

The supported peptide having the terminal amine protected by an Fmoc group is dissolved in acetonitrile then piperidine (10 to 20% by volume) is added. The medium is stirred for 15 minutes at AT before evaporating the solvents. The residue is washed with ether then dissolved in DCM. This phase is washed three times with one-tenth by volume of an aqueous solution of HPF₆. The organic phase is dried over Na₂SO₄, filtered and the DCM is evaporated off.

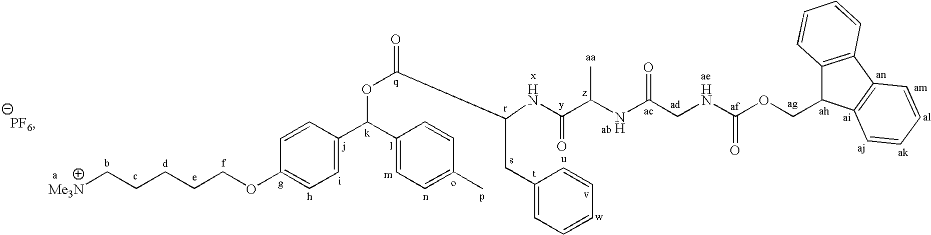

### [Fmoc-Gly-Ala-Phe-HTMPPTMA][PF₆]

Procedure: cf procedures 8′ using [Fmoc-Ala-Phe-HTMPPTMA][PF₆] then 11 using the [Ala-Phe-HTMPPTMA][PF₆] formed and Fmoc-glycine. The yield by mass is 98% over two stages. The product is contaminated with 4% [HTMPPTMA][PF₆] (cleavage by formation of DKP in the deprotected supported dipeptide stage).

viscous yellow oil

NMR¹H (300 MHz, acetone d₆): δ(Hₐ)=3.34 (s, 9H); δ(H_{b})=3.56 (m, 2H); δ(H_{c})=1.86 (m, 2H); δ(H_{d})=1.58 (m, 2H); δ(Hₑ)=2.07 (m, 2H); δ(H_{f+ad})=3.80-4.07 (m, 2H+2H); δ(Hₕ₊ₐₑ)=6.80-6.95 (m, 2H+1H); δ(H_{i+m+n+u+v+w+x+ab+al+ak})=7.05-7.54 (m, 2H+2H+2H+2H+2H+1H+1H+1H+2H+2H); δ(Hₖ)=6.77 (s, 1H); δ(Hₚ)=2.29 (m, 3H); δ(Hᵣ)=4.81 (m, 1H); δ(H_{s+s′})=2.90-3.25 (m, 1H+1H); δ(H_{z})=4.48 (m, 1H); δ(Hₐₐ)=1.24 (d, J=6.9, 3H); δ(H_{ag+ah})=4.16-4.40 (m, 2H+1H); δ(Hₐⱼ)=7.69 (m, 2H); δ(Hₐₘ)=7.87 (d, J=7.4, 2H).

NMR¹³C (75 MHz, acetone d₆): δ(Cₐ)=52.67; δ(C_{b})=66.71; δ(Cᵣ)=22.68; δ(C_{d})=22.35; δ(Cₑ)=28.43; δ(C_{f})=67.27; δ(C_{g})=158.79; δ(Cₕ)=114.37; δ(Cᵢ)=129.08; δ(Cⱼ)=132.50; δ(Cₖ)=77.46; δ(Cₗ)=136.69; δ(Cₘ)=126.88; δ(Cₙ)=129.33; δ(Cₒ)=137.75; δ(Cₚ)=20.34; δ(C_{q})=170.27; δ(Cᵣ)=54.09; δ(Cₛ)=37.13; δ(Cᵣ)=137.30; δ(Cᵤ)=128.72; δ(Cᵥ)=128.59; δ(C_{w})=126.69; δ(C_{y})=172.25; δ(C_{z})=48.70; δ(Cₐₐ)=17.56; δ(C_{ac})=169.25; δ(C_{ad})=44.28; δ(C_{af})=157.01; δ(C_{ag})=66.40; δ(Cₐₕ)=47.04; δ(Cₐᵢ)=144.21; δ(Cₐⱼ)=125.32; δ(Cₐₖ)=127.20; δ(Cₐₗ)=128.19; δ(Cₐₘ)=120.04; δ(Cₐₙ)=141.21.

HRMS (ESI) of (C₅₁H₅₉N₄O₇): [C⁺] m/z_{theoretical}=839.4383; m/zₑₓₚₑᵣᵢₘₑₙₜₐₗ=839.4378.

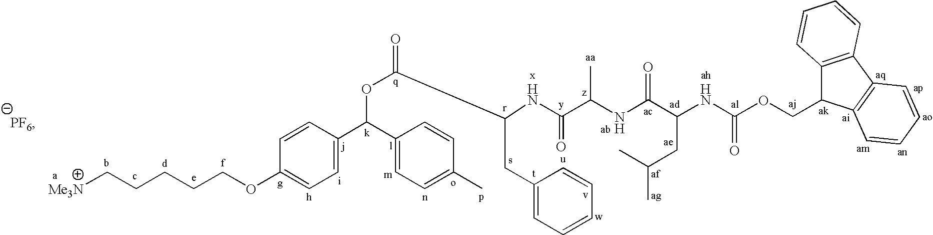

### [Fmoc-Leu-Ala-Phe-HTMPPTMA][PF₆]

Procedure: cf procedures 8′ using [Fmoc-Ala-Phe-HTMPPTMA][PF₆] then 11 using the [Ala-Phe-HTMPPTMA][PF₆] formed and Fmoc-leucine. The yield by mass is 98% over two stages. The product is contaminated with 4% [HTMPPTMA][PF₆] (cleavage by formation of DKP in the deprotected supported dipeptide stage).

viscous yellow oil

NMR¹H (300 MHz, acetone d₆): δ(Hₐ)=3.34 (s, 9H); δ(H_{b})=3.58 (m, 2H); δ(H_{c+d+e+ae+af})=1.50-2.08 (m, 2H+2H+2H+2H+1H); δ(H_{f})=4.01 (m, 2H); δ(Hₕ)=6.88 (m, 2H); δ(H_{i+m+n+u+v+w+x+ab+an+ao})=7.02-7.60 (m, 2H+2H+2H+2H+2H+1H+1H+1H+2H+2H); δ(H_{k±ah})=6.67-6.77 (m, 1H+1H); δ(Hₚ)=2.30 (m, 3H); δ(Hᵣ)=4.81 (m, 1H); δ(H_{s+s′})=2.90-3.23 (m, 1H+1H); δ(H_{z+ad+aj+ak})=4.12-4.50 (m, 1H+1H+2H+1H); δ(Hₐₐ)=1.25 (d, J=7.0, 3H); δ(H_{ag})=0.92 (d, J=6.3, 3H); δ(H_{ag′})=0.94 (d, J=6.4, 3H); δ(Hₐₘ)=7.70 (m, 2H); δ(Hₐₚ)=7.87 (d, J=7.4, 2H).

NMR¹³C (75 MHz, acetone d₆): δ(Cₐ)=52.68 (t, J_{C-N}=3.7); δ(C_{b})=66.43; δ(C_{c})=22.73; δ(C_{d})=22.36; δ(Cₑ)=28.46; δ(C_{f})=67.25; δ(C_{g})=158.82; δ(Cₕ)=114.33; δ(Cᵢ)=129.02; δ(Cⱼ)=132.50; δ(Cₖ)=77.39; δ(Cₗ)=136.69; δ(Cₘ)=126.948; δ(Cₙ)=129.28; δ(Cₒ)=137.93; δ(Cₚ)=21.10; δ(C_{q})=170.19; δ(Cᵣ)=53.94; δ(Cₛ)=37.26; Kₜ)=137.25; δ(Cᵤ)=128.37; δ(Cᵥ)=128.16; δ(C_{w})=126.68; δ(C_{y})=172.12; δ(C_{z})=48.66; δ(Cₐₐ)=17.69; δ(C_{ac})=172.32; δ(C_{ad})=53.73; δ(Cₐₑ)=41.13; δ(C_{af})=24.57; δ(C_{ag})=20.31; δ(Cₐᵢ)=156.48; δ(Cₐⱼ)=66.43; δ(Cₐₖ)=47.14; δ(Cₐᵢ)=144.17; δ(Cₐₘ)=126.35; δ(Cₐₙ)=126.16; δ(Cₐₒ)=127.74; δ(Cₐₚ)=120.01; δ(C_{aq})=141.22.

HRMS (ESI) of (C₅₅H₆₇N₄O₇): [C⁺] m/z_{theoretical}=895.5009; m/zₑₓₚₑᵣᵢₘₑₙₜₐₗ=895.5006.

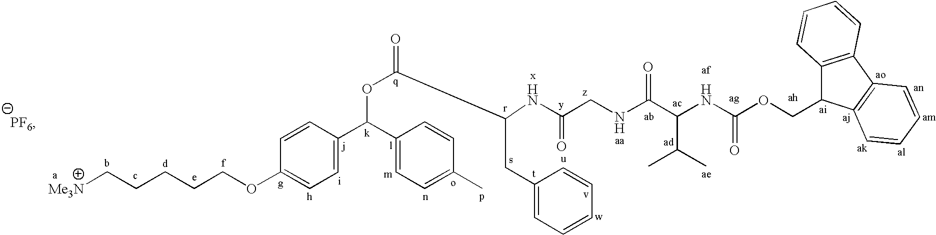

### [Fmoc-Val-Gly-Phe-HTMPPTMA][PF₆]

Procedure: cf procedures 8′ using [Fmoc-Gly-Phe-HTMPPTMA][PF₆] then 11 using the [Gly-Phe-HTMPPTMA][PF₆] formed and Fmoc-valine. The yield by mass is 83% over two stages. The product is contaminated with 5% [HTMPPTMA][PF₆] (cleavage by formation of DKP in the deprotected supported dipeptide stage).

viscous yellow oil

NMR¹H (300 MHz, acetone d₆): δ(Hₐ)=3.34 (s, 9H); δ(H_{b})=3.56 (m, 2H); δ(H_{c})=1.86 (m, 2H); δ(H_{d})=1.58 (m, 2H); δ(H_{e+ad})=2.07 (m, 2H+1H); δ(H_{f+z})=3.70-4.05 (m, 2H+2H); δ(Hₕ)=6.86 (m, 2H); δ(H_{i+m+n+u+v+w+aa+al+am})=7.05-7.54 (m, 2H+2H+2H+2H+2H+1H+1H+2H+2H); δ(H_{k+af})=6.71-6.82 (m, 1H+1H); δ(Hₚ)=2.29 (m, 3H); δ(Hᵣ)=4.80 (m, 1H); δ(H_{s+s′})=2.95-3.20 (m, 1H+1H); δ(Hₓ₊ₐₖ)=7.53-7.73 (m, 1H+2H); δ(H_{ac+ah+ai})=4.18-4.55 (m, 1H+2H+1H); δ(Hₐₑ)=0.99 (d, J=6.7, 6H); δ(Hₐₙ)=7.86 (d, J=7.5, 2H).

NMR¹³C (75 MHz, acetone d₆): δ(Cₐ)=52.65; δ(C_{b})=66.59; δ(Cᵣ)=22.70; δ(C_{d})=22.36; δ(Cₑ)=28.46; δ(C_{f})=67.24; δ(C_{g})=158.77; δ(Cₕ)=114.32; δ(Cᵢ)=129.03; δ(Cⱼ)=132.50; δ(Cₖ)=77.38; δ(Cₗ)=136.73; δ(Cₘ)=126.87; δ(Cₙ)=129.30; δ(Cₒ)=137.75; δ(Cₚ)=20.33; δ(C_{q})=170.27; δ(Cᵣ)=54.06; δ(Cₛ)=37.36; δ(Cᵣ)=137.25; δ(Cₙ)=128.56; δ(Cᵥ)=128.40; δ(C_{w})=126.72; δ(C_{y})=171.93; δ(C_{z})=42.37; δ(C_{ab})=168.90; δ(Cₐᵣ)=61.08; δ(C_{ad})=30.50; δ(Cₐₑ)=17.78; δ(C_{ae′})=18.93; δ(C_{ag})=158.09; δ(Cₐₕ)=66.40; δ(Cₐᵢ)=47.10; δ(Cₐⱼ)=144.12; δ(Cₐₖ)=125.33; δ(Cₐᵢ)=127.16; δ(Cₐₘ)=127.76; δ(Cₐₙ)=120.01; δ(Cₐₒ)=141.20.

HRMS (ESI) of (C₅₃H₆₃N₄O₇): [C⁺] m/z_{theoretical}=867.4696; m/zₑₓₚₑᵣᵢₘₑₙₜₐₗ=867.4693.

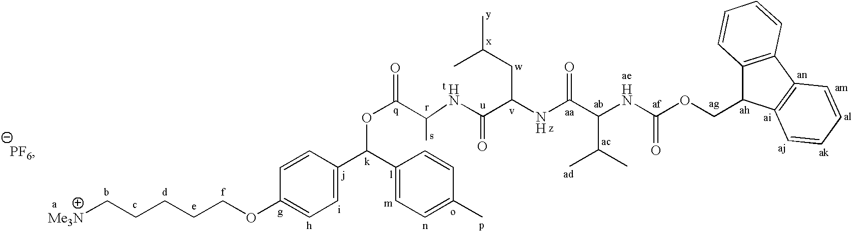

### [Fmoc-Val-Leu-Ala-HTMPPTMA][PF₆]

Procedure: cf procedures 8′ using [Fmoc-Leu-Ala-HTMPPTMA][PF₆] then 11 using the [Leu-Ala-HTMPPTMA][PF₆] formed and Fmoc-valine. The yield by mass is 91% over two stages. The product is contaminated with 3% [HTMPPTMA][PF₆] (cleavage by formation of DKP in the deprotected supported dipeptide stage).

viscous yellow oil

NMR¹H (300 MHz, acetone d₆): δ(Hₐ)=3.34 (s, 9H); δ(H_{b})=3.57 (m, 2H); δ(H_{c+d+w})=1.52-1.67 (m, 2H+2H+2H); δ(Hₑ)=1.86 (m, 2H); δ(H_{f})=4.00 (m, 2H); δ(Hₕ)=6.88 (dd, J₁=8.8, J₂=2.8, 2H); δ(Hᵢ₊ₘ₊ₙ₊ₐₖ₊ₐₗ)=7.11-7.49 (m, 2H+2H+2H+2H+2H); δ(Hₖ)=6.77 (s, 1H); δ(Hₚ)=2.30 (d, J=2.9, 3H); δ(H_{r+ac+ag+ah})=4.09-4.44 (m, 1H+1H+2H+1H); δ(Hₛ)=1.37 (dd, J₁=7.1, J₂=2.9, 3H); δ(Hₜ₊ₐⱼ)=7.66-7.78 (m, 1H+2H); δ(H_{v+ab})=4.50-4.61 (m, 1H+1H); δ(Hₓ)=1.70 (m, 1H); δ(H_{y})=0.96 (d, J=6.6, 3H); δ(H_{y′})=0.99 (d, J=5.5, 3H); δ(H_{z})=7.57 (m, 1H); δ(H_{ad})=0.85 (d, J=6.3, 3H); δ(H_{aof})=0.86 (d, J=6.4, 3H); δ(Hₐₑ)=6.72 (m, 1H); δ(Hₐₘ)=7.87 (d, J=7.5, 2H).

NMR¹³C (75 MHz, acetone d₆): δ(Cₐ)=52.70 (t, J_{C-N}=3.7); δ(C_{b})=66.48; δ(C_{c})=22.64; δ(C_{d})=22.37; δ(Cₑ)=28.50; δ(C_{f})=67.19; δ(C_{g})=158.72; δ(Cₕ)=114.28; δ(Cᵢ)=128.34; δ(Cⱼ)=132.76; δ(Cₖ)=77.05; δ(Cₗ)=137.23; δ(Cₘ)=126.69; δ(Cₙ)=129.01; δ(Cₒ)=137.93; δ(Cₚ)=21.17; δ(C_{q})=171.85; δ(Cᵣ)=48.19; δ(Cₛ)=16.85; δ(Cᵤ)=171.37; δ(Cᵥ)=51.23; δ(C_{w})=41.11; δ(Cₓ)=24.39; δ(C_{y})=20.23; δ(Cₐₐ)=171.37; δ(C_{ab})=60.62; δ(C_{ac})=30.91; δ(C_{ad})=17.63; δ(C_{aof})=18.91; δ(C_{af})=156.61; δ(C_{ag})=66.48; δ(Cₐₕ)=47.13; δ(Cₐᵢ)=144.19; δ(Cₐⱼ)=126.32; δ(Cₐₖ)=126.69; δ(Cₐₗ)=128.32; δ(Cₐₘ)=119.95; δ(Cₐₙ)=141.19.

HRMS (ESI) of (C₅₁H₆₇N₄O₇): [C⁺] m/z_{theoretical}=847.5010; m/Zₑₓₚₑᵣᵢₘₑₙₜₐₗ=847.5024.

2.3.4. Cleavage of the Supported Peptides.

General procedure 12 for the cleavage of the supported peptides:

1.0 eq. of supported peptide having the deprotected amine [AAₙ- . . . AA₁-HTMPPTMA][PF₆] is dissolved in methanol (concentration of 0.1 mol/L) then 1% of a 60% aqueous solution of HPF₆ is added. The mixture is taken to reflux for one hour then the methanol is evaporated off. Dichloromethane and water are added to the residue. Evaporation of the solvent from each phase makes it possible to isolate both the peptide (dissolved in aqueous phase) and [HTMPPTMA][PF₆] (dissolved in organic phase).

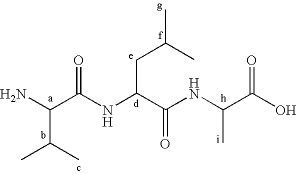

### Val-Leu-Ala

Procedure: cf procedures 8′ using [Fmoc-Val-Leu-Ala-HTMPPTMA][PF₆] then 12 using [Val-Leu-Ala-HTMPPTMA][PF₆]. The yield is 85%.

colourless oil

NMR¹H (300 MHz, D₂O): δ(Hₐ)=3.72 (d, J=5.81H); δ(H_{b})=2.11 (m, 1H); δ(H_{c})=0.92 (t, J=6.5, 6H); δ(H_{d})=4.33 (t, J=7.3, 1H); δ(H_{e+e′+f})=1.46-1.54 (m, 1H+1H+1H); δ(H_{g})=0.82 (dd, J₁=6.3, J₂=6.0, 6H); δ(Hₕ)=4.03 (q, J=7.2, 1H); δ(H₁)=1.23 (d, J=7.2, 3H).

2.3.5. Convergent Synthesis

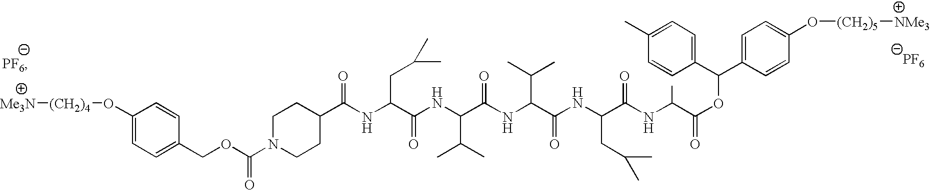

### [HMPhBTMA-Aiso-Leu-Val-Val-Leu-Ala-CTMPTTMA]([PF₆])₂

Procedure:

1.0 eq. of [Val-Leu-Ala-CTMPTTMA][PF₆] and 1.0 eq. of [HMPhBTMA-Aiso-Leu-Val][PF₆] are dissolved in acetonitrile then 1.5 eq. of TEA, HOBt and carbodiimide are added. The reaction medium is stirred overnight at AT. The acetonitrile is evaporated off. The residue obtained is then washed with ether which causes its precipitation. The solid obtained is washed three times with water then three times with an aqueous solution of HPF₆ (1<pH<2) before being dried overnight in a desiccator. The two starting supported peptides are not visible in the mass spectrum

The yield is 50%.

cream solid

HRMS of (C₆₈H₁₀₈N₈O₁₁): [C⁺⁺] m_{theoretical}=1212.8138; m/zₑₓₚₑᵣᵢₘₑₙₜₐₗ=606.4063.

1. Use of a salt with a dedicated task of formula (I): A⁺-L-R—OY, X⁻ as soluble support for peptide synthesis, in which:
X⁻ represents a functional or non-functional anion, chosen in particular from Cl, Br⁻, I⁻, BF₄⁻, CF₃SO₃⁻, N(SO₂CF₃)₂⁻, PF₆⁻, CH₃CO₂⁻, CF₃CO₂⁻, R_{α}CO₂⁻, R_{F}CO₂⁻, R_{α}SO₃⁻, R_{F}SO₃⁻, R_{α}SO₄⁻, (R_{α})₃₋ₓPO₄^{x-}, x representing an integer equal to 1, 2 or 3, AlCl₄⁻, SnCl₃⁻, ZnCl₃⁻, R_{α} representing an alkyl group comprising 1 to 20 carbon atoms, R_{F} representing a perfluoroalkyl group comprising 1 to 20 carbon atoms,
Y represents:
either a hydrogen atom, the salt of formula (I) then comprising a cation functionalized by an alcohol function and corresponding to the following formula (I_{D}): A⁺-L-R—OH, X⁻,
or a —COOR₁ group, R₁ representing an alkyl group comprising 1 to 20 carbon atoms or an aryl group comprising 6 to 30 carbon atoms, or a perfluoroalkyl group comprising 1 to 20 carbon atoms, said alkyl or aryl groups being optionally functionalized, R₁ representing in particular —CHCl— CCl₃ or 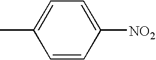
the salt of formula (I) then comprising a cation functionalized by a mixed carbonate function and corresponding to the following formula (I_{I}): 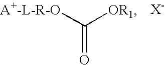
A⁺ represents a cationic entity, in particular chosen from pyridinium, imidazolium, ammonium, phosphonium or sulphonium cations, cyclic or non-cyclic, substituted or non-substituted, and preferably ammonium or phosphonium,
L represents an arm, in particular a linear or branched alkyl group, or aralkyl or alkaryl comprising 3 to 20 carbon atoms,
R represents a group chosen from the following groups:
a group of formula —C(Rₐ)(R_{b})—, Rₐ and R_{b} representing independently of one another a hydrogen atom or a linear or branched alkyl group, comprising 1 to 20 carbon atoms, the group of formula —C(Rₐ)(R_{b})— preferably representing a —CH₂—, —CH(Me)- or —C(Me)₂- group,
a group of formula -T-Ar₁—CH(R_{c})—, in which:
T is chosen from one of the following groups: CH₂, O, S and NR_{d}, R_{d} representing a hydrogen atom or a linear or branched alkyl group, comprising 1 to 20 carbon atoms,
Ar₁ represents an aromatic group of the following formula: 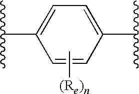
n representing an integer equal to 0, 1, 2, 3 or 4,
Rₑ representing either a linear or branched alkyl group, comprising 1 to 12 carbon atoms, in particular a methyl group, or an alkoxy group comprising 1 to 12 carbon atoms, in particular a methoxy, ethoxy, propyloxy, isopropyloxy, butyloxy, isobutyloxy or tertiobutyloxy group,
R_{c} represents either a hydrogen atom, or a linear or branched alkyl group, comprising 1 to 20 carbon atoms, or an aromatic group Arₑ of the following formula: 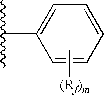
m representing an integer equal to 1, 2, 3, 4 or 5,
R_{f} representing either a linear or branched alkyl group, comprising 1 to 12 carbon atoms, in particular a methyl group, or an alkoxy group comprising 1 to 12 carbon atoms, in particular a methoxy, ethoxy, propyloxy, isopropyloxy, butyloxy, isobutyloxy or tertiobutyloxy group.

2. Use according to claim 1, for peptide synthesis, of azapeptides or pseudopeptides, said peptides, azapeptides or pseudopeptides comprising at least one peptide bond and/or at least one azapeptide bond and/or at least one pseudopeptide bond, and optionally comprising at least one α-hydrazino acid, α-amino acid or ω-amino acid unit, in particular β-amino acid or γ-amino acid, cyclic or linear.

3. Use according to claim 1, for the grafting of at least an amino acid
of formula HOOC—[CH(R′)]ₚ—NHGP, onto a compound of formula (I_{D}) as defined in claim 1,
p representing an integer varying from 1 to 20,
R′ representing an amino acid residue,
GP representing a protective group of the amine function, with the exception of Boc, in particular Fmoc, Cbz, Z, SO₂R_{g}, R_{g} representing a linear or branched alkyl group comprising 1 to 20 carbon atoms, a substituted or non-substituted aryl group, a perfluoroalkyl group comprising 1 to 20 carbon atoms,
to obtain a compound of the following formula: 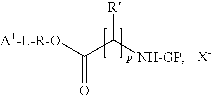
A⁺, L and R being as defined in claim 1,
p, R′ and GP being as defined above,
or of formula R₂—NH—[CH(R′)]ₚ—COOR₃, onto a compound of formula (I_{I}) as defined in claim 1,
p representing an integer varying from 1 to 20,
R′ representing an amino acid residue,
R₂ representing a linear or branched alkyl group, comprising 1 to 20 carbon atoms and being able to form a ring with the R′ group, the nitrogen atom carrying the group R₂ and the carbon atom carrying the R′ group, said ring comprising 3 to 20 members, in particular 5 or 6 members, and
R₃ representing a hydrogen atom or a protective group of the terminal acid function of the amino acid, and being chosen from one of the following groups: a linear or branched alkyl group, comprising 1 to 20 carbon atoms, in particular methyl or tertiobutyl, a benzyl group or an Si(ORₕ)₃, Rₕ group representing a linear or branched alkyl group of 1 to 20 carbon atoms, and representing in particular a tertiobutyl group,
to obtain a compound of the following formula: 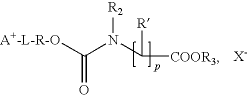
A⁺, L and R being as defined in claim 1,
p, R₂, R′ and R₃ being as defined above.

4. Use according to any one of claims 1 to 3, of a salt with a dedicated task of formula 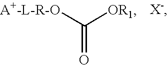
for reverse-route peptide synthesis, in which:
A⁺, X⁻ and L are as defined in claim 1,
R₁ represents in particular a —CHCl—CCl₃ or 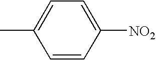
group
R represents a group of formula —C(Rₐ)(R_{b})—, Rₐ and R_{b} representing independently of one another a hydrogen atom or a linear or branched alkyl group, comprising 1 to 20 carbon atoms, the group of formula —C(Rₐ)(R_{b})— preferably representing a —CH₂—, —CH(Me)— or —C(Me)₂- group.

5. Use according to any one of claims 1 to 3, of a salt with a dedicated task of formula A⁺-L-R—OH, X⁻, for direct route peptide synthesis, in which:
A⁺, X⁻ and L are as defined in claim 1,
R represents a group of formula -T-Ar₁-CH(R_{c})—, in which:
T is chosen from one of the following groups: CH₂, O, S and NR_{d}, in particular O, R_{d} representing a hydrogen atom or a linear or branched alkyl group, comprising 1 to 20 carbon atoms,
Ar₁ represents an aromatic group of the following formula: 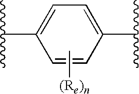
n representing an integer equal to 0, 1, 2, 3 or 4,
Rₑ representing either a linear or branched alkyl group, comprising 1 to 20 carbon atoms, in particular a methyl group, or an alkoxy group comprising 1 to 20 carbon atoms, in particular a methoxy, ethoxy, propyloxy, isopropyloxy, butyloxy, isobutyloxy or tertiobutyloxy group,
R_{c} represents either a hydrogen atom, or a linear or branched alkyl group, comprising 1 to 20 carbon atoms, or an aromatic group Arₑ of the following formula: 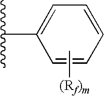
m representing an integer equal to 1, 2, 3, 4 or 5,
R_{f} representing either a linear or branched alkyl group, comprising 1 to 20 carbon atoms, in particular a methyl group, or an alkoxy group comprising 1 to 20 carbon atoms, in particular a methoxy, ethoxy, propyloxy, isopropyloxy, butyloxy, isobutyloxy or tertiobutyloxy group.

6. Use according to claim 1, for convergent route peptide synthesis of a salt with a dedicated task A⁺-L-R—OY, X⁻ of formula (I) as defined in claim 1, and of a salt with a dedicated task of formula Aᵢ⁺-Lᵢ-Rᵢ—OH, Xᵢ⁻, the elements A⁺, L, R, Y and X⁻ being as defined in claim 1, and the elements Aᵢ⁺-Lᵢ-Rᵢ, and Xᵢ⁻ having the definitions given in claim 1 with respect to A⁺, L, R and X⁻, A⁺-L-R and Aᵢ⁺-Lᵢ-Rᵢ respectively being able to be identical or different.

7. Use according to any one of claims 1 to 6, characterized in that A⁺ is chosen from the quaternary ammonium cations, cyclic or non-cyclic.

8. Use according to any one of claims 1 to 7, characterized in that L represents a linear alkyl chain comprising 4 or 5 carbon atoms.

9. Use according to any one of claims 1 to 8, characterized in that the anion X⁻ is PF₆⁻ or NTf₂⁻.

10. Use according to claim 4, comprising the use of a salt with a dedicated task in which the cation corresponds to one of the following formulae:

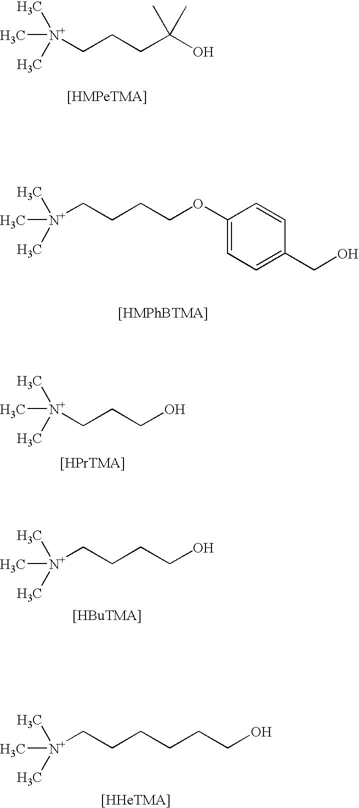

11. Use according to claim 5, comprising the use of a salt with a dedicated task in which the cation corresponds to the following formula:

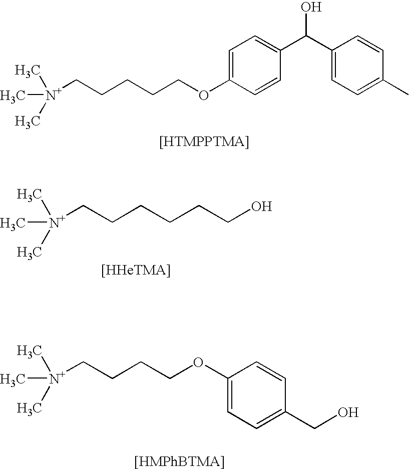

12. Use according to any one of claims 1 to 11, characterized in that the salt with a dedicated task is:
either solubilized in a standard organic solvent such as dichloromethane, tetrahydrofuran, dioxane, acetonitrile, propionitrile, dimethylformamide, dimethylacetamide, N-methyl-pyrrolidone, acetone, toluene, chlorobenzene, dichlorobenzene, nitromethane, nitroethane, or a mixture of these solvents,
or solubilized in an ionic liquid matrix, preferably trimethylbutylammonium triflimidide or [tmba][NTf₂], 1-ethyl-3-methylimidazolium triflimidide or [emim][NTf₂], 1-butyl-3-methylimidazolium triflimidide or [bmim][NTf₂] or any other combination of onium cation and of liquid anion at a temperature less than or equal to 100° C., preferably 50° C.,
or solubilized in a mixture comprising an organic solvent and an ionic liquid matrix as defined above.

13. Use according to any one of claims 1 to 3, 5 and 7 to 12, for direct route peptide synthesis, characterized in that the salt with a dedicated task is in solution in an organic solvent.

14. Use according to any one of claims 1 to 3, 5 and 7 to 12, for direct route peptide synthesis, characterized in that the salt with a dedicated task is solubilized and immobilized in an ionic liquid matrix A₂⁺, X₂⁻,
the cation A₂⁺ being chosen from the imidazolium, pyridinium, substituted or non-substituted, ammonium, phosphonium, sulphonium cations or any other optionally functionalized onium cation, and
the anion X₂⁻ being chosen from Cl⁻, Br⁻, I⁻, F⁻, BF₄⁻, CF₃SO₃⁻, N(SO₂CF₃)₂⁻, PF₆⁻, CH₃CO₂⁻, CF₃CO₂⁻, R_{α}CO₂⁻, R_{F}CO₂⁻, R_{α}SO₃⁻, R_{F}SO₃, R_{α}SO₄⁻, (R_{α})₃₋ₓPO₄^{x-}, x representing an integer equal to 1, 2 or 3, AlCl₄⁻, SnCl₃⁻, ZnCl₃⁻, R_{α} representing an alkyl group comprising 1 to 20 carbon atoms, R_{F} representing a perfluoroalkyl group comprising 1 to 20 carbon atoms.

15. Use according to any one of claims 1 to 4 and 7 to 12, for reverse-route peptide synthesis, characterized in that the salt with a dedicated task is in solution in an organic solvent.

16. Use according to any one of claims 1 to 4 and 7 to 12, for reverse-route peptide synthesis, characterized in that the salt with a dedicated task is solubilized and immobilized in an ionic liquid matrix A₂⁺, X₂⁻,
the cation A₂⁺ being chosen from the imidazolium, pyridinium, substituted or non-substituted, ammonium, phosphonium, sulphonium cations or any other optionally functionalized onium cation, and
the anion X₂⁻ being chosen from Cl⁻, Br⁻, I⁻, F⁻, BF₄⁻, CF₃SO₃⁻, N(SO₂CF₃)₂⁻, PF₆⁻, CH₃CO₂⁻, CF₃CO₂⁻, R_{α}CO₂⁻, R_{F}CO₂⁻, R_{α}SO₃⁻, R_{F}SO₃⁻, R_{α}SO₄⁻, (R_{α})₃₋ₓPO₄^{x-}, x representing an integer equal to 1, 2 or 3, AlCl₄⁻, SnCl₃⁻, ZnCl₃⁻, R_{α} representing an alkyl group comprising 1 to 20 carbon atoms, R_{F} representing a perfluoroalkyl group comprising 1 to 20 carbon atoms.

17. Use according to any one of claims 1 to 3 and 6 to 12, for peptide synthesis by convergent route, characterized in that the salts with a dedicated task are in solution in an organic solvent.

18. Use according to any one of claims 1 to 3 and 6 to 12, for peptide synthesis by convergent route, characterized in that the salts with a dedicated task are solubilized and immobilized in an ionic liquid matrix A₂⁺, X₂⁻,
the cation A₂⁺ being chosen from the imidazolium, pyridinium, substituted or non-substituted, ammonium, phosphonium, sulphonium cations or any other optionally functionalized onium cation, and
the anion X₂⁻ being chosen from Cl⁻, Br⁻, I⁻, F⁻, BF₄⁻, CF₃SO₃⁻, N(SO₂CF₃)₂⁻, PF₆⁻, CH₃CO₂⁻, CF₃CO₂⁻, R_{α}CO₂⁻, R_{F}CO₂⁻, R_{α}SO₃⁻, R_{F}SO₃⁻, R_{F}SO₄⁻, (R_{α})₃₋ₓPO₄^{x-}, x representing an integer equal to 1, 2 or 3, AlCl4⁻, SnCl₃⁻, ZnCl₃⁻, R_{α} representing an alkyl group comprising 1 to 20 carbon atoms, R_{F} representing a perfluoroalkyl group comprising 1 to 20 carbon atoms.

19. Process for direct-route peptide synthesis (C→N) on a support as defined according to any one of claims 1 to 18, for the preparation of a peptide of the following formula (II): 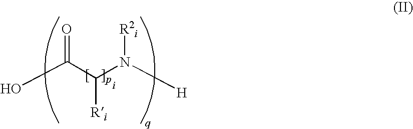
in which:
i is an integer varying from 1 to q,
q is an integer varying from 1 to 20,
pᵢ is an integer varying from 1 to 20,
R′ᵢ represents an amino acid residue,
Rᵢ² represents H or a linear or branched alkyl group, comprising 1 to 20 carbon atoms and being able to form a ring with the R′ᵢ group, the nitrogen atom carrying the group Rᵢ² and the carbon atom carrying the R′ᵢ group, said ring comprising 3 to 20 members, in particular 5 or 6 members,
said process comprising the following stages:
f) a stage of grafting of an amino acid HOOC—[CH(R′₁)]ₚ_{₁}—N(R₁²)-GP,
R′₁, R₁² and p₁ being as defined above, and GP representing a protective group of the amine function, with the exception of Boc, in particular Fmoc, Cbz, Z, SO₂R_{g}, R_{g} representing a linear or branched alkyl group comprising 1 to 20 carbon atoms, a substituted or non-substituted aryl group, a perfluoroalkyl group comprising 1 to 20 carbon atoms,
on a soluble support of the following formula (I_{D}): A⁺-L-R—OH, X⁻,
A⁺, L, R and X⁻ being as defined in claim 1,
in order to obtain the product of the following formula (II-1): 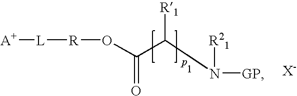
g) a stage of deprotection of the product of formula (II-1) as obtained at the end of the preceding stage in order to obtain the deprotected product of the following formula (III-1): 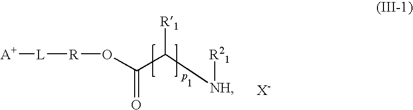
h) the sequential repetition of Stages a) and b) of grafting and deprotection up to the obtaining of the protected supported peptide of the following formula (II-q): 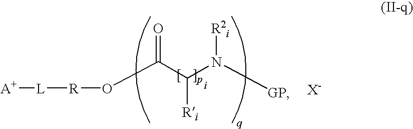
i) a stage of deprotection of the protected supported peptide of formula (II-q) as obtained at the end of the preceding stage in order to obtain the deprotected supported peptide of the following formula (III-q): 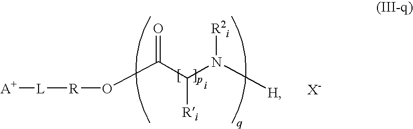
j) and a stage of cleavage from the support in order to obtain the abovementioned peptide of formula (II) and optionally to recycle the support of formula (I_{D}) A⁺-L-R—OH, X⁻,
the order of Stages d) and e) being able to be reversed.

20. Process for reverse route peptide synthesis (N→C) on a support as defined according to any one of claims 1 to 18, for the preparation of a peptide of the following formula (IV): 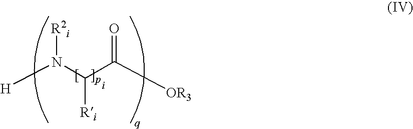
in which:
i is an integer varying from 1 to q,
q is an integer varying from 1 to 20,
p, is an integer varying from 1 to 20,
R′ᵢ represents an amino acid residue,
Rᵢ² represents H or a linear or branched alkyl group, comprising 1 to 20 carbon atoms and being able to form a ring with the R′, group, the nitrogen atom carrying the group Rᵢ² and the carbon atom carrying the R′, group, said ring comprising 3 to 20 members, in particular 5 or 6 members,
R₃ representing a hydrogen atom or a protective group of the terminal acid function of the amino acid, and being chosen from one of the following groups: a linear or branched alkyl group, comprising 1 to 20 carbon atoms, in particular methyl or tertiobutyl, a benzyl group or an Si(ORₕ)₃ group, Rₕ representing a linear or branched alkyl group of 1 to 20 carbon atoms, and representing in particular a tertiobutyl group,
said process comprising the following stages:
g) a stage of reaction of a compound of the following formula: 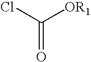
R₁ being as defined in claim 1, and representing in particular —CHCl—CCl₃ or 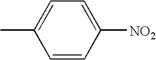
on a soluble support of the following formula (I_{D}):
A⁺-L-R—OH, X⁻
A⁺, L, R and X⁻ being as defined in claim 1,
in order to obtain a soluble support of the following formula (I_{I}): 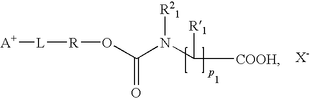
A⁺, L, R, R₁ and X⁻ being as defined above,
h) a stage of grafting of an amino acid NH(R₁²)-[CH(R′₁)]ₚ_{₁}—COOR_{S}, onto a soluble support of formula (I_{I}) as obtained at the end of the preceding stage,
p₁, R₁² and R′₁ being as defined above,
R₃ being as defined in claim 3,
in order to obtain a compound of the following formula (IV-1): 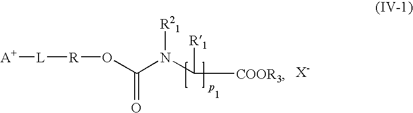
X⁻, A⁺, L, R, p₁, R′₁ and R₃ being as defined above,
i) a stage of optional deprotection of the product of formula (IV-1) as obtained at the end of the preceding stage in order to obtain the deprotected product of the following formula (V-1): 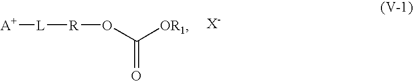
j) the sequential repetition of Stages b) and c) of grafting and deprotection up to the obtaining of the supported peptide of the following formula (IV-q): 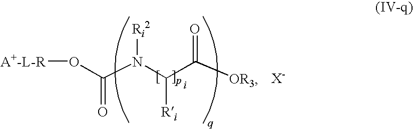
k) a stage of optional deprotection of the supported peptide of formula (IV-q) as obtained at the end of the preceding stage in order to obtain the deprotected supported peptide of the following formula (V-q): 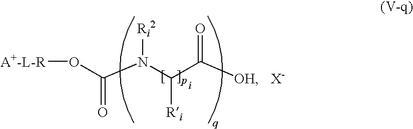
l) and a stage of cleavage from the support in order to obtain the abovementioned peptide of formula (IV) and optionally to recycle the support of formula (I_{D}) A⁺-L-R—OH, X⁻,
the order of Stages e) and f) being able to be reversed.

21. Process for convergent route peptide synthesis on a support as defined according to any one of claims 1 to 18, for the preparation of a peptide of the following formula (VI): 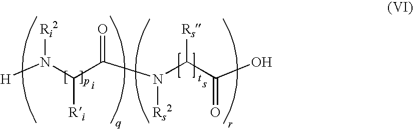
in which:
i is an integer varying from 1 to q,
q is an integer varying from 1 to 20,
pᵢ is an integer varying from 1 to 20,
R′ᵢ represents an amino acid residue,
Rᵢ² represents H or a linear or branched alkyl group, comprising 1 to 20 carbon atoms and being able to form a ring with the R′, group, the nitrogen atom carrying the Rᵢ² group and the carbon atom carrying the R′, group, said ring comprising 3 to 20 members, in particular 5 or 6 members,
s is an integer varying from 1 to r,
r is an integer varying from 1 to 20,
tₛ is an integer varying from 1 to 20,
R″ₛ represents an amino acid residue,
Rₛ² represents H or a linear or branched alkyl group, comprising 1 to 20 carbon atoms and being able to form a ring with the R″, group, the nitrogen atom carrying the group R₅² and the carbon atom carrying the R″, group, said ring comprising 3 to 20 members, in particular 5 or 6 members,
said process comprising the following stages:
c) the reaction of a supported peptide obtained by reverse route peptide synthesis of the following formula (VII-I): 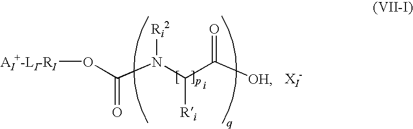
A_{I}⁺, L_{I}, R_{I} and X_{I}⁻ corresponding to the same definition as that given for A⁺, L, R and X⁻ in claim 1,
i, q, Rᵢ², p, and R′ᵢ being as defined above,
with a supported peptide obtained by direct route synthesis of the following formula (VII-D): 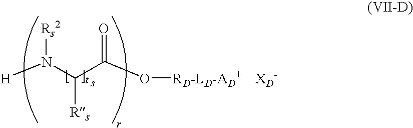
A_{D}⁺, L_{D}, R_{D} and X_{D}⁻ corresponding to the same definition as that given for A⁺, L,
R and X⁻ in claim 1,
A_{D}⁺-L_{D}-R_{D} and A_{I}⁺-L_{I}-R_{I} being able to be identical or different,
and X_{D}⁻ and X_{I}⁻ being able to be identical or different,
s, r, Rₛ², tₛ and R″ₛ being as defined above,
in order to obtain a bi-supported peptide of the following formula (VIII): 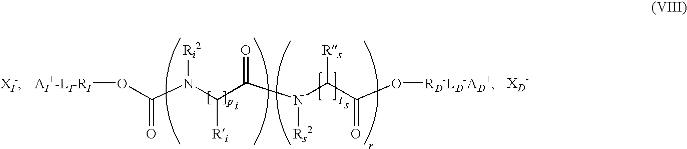
d) and a stage of cleavage of the product of formula (VIII) in order to obtain the abovementioned peptide of formula (VI), and optionally to recycle the supports of the following formula: A_{D}⁺-L_{D}-R_{D}—OH, X_{D}⁻, and A_{I}⁺-L_{I}-R_{I}—OH_{I}⁻.

22. Peptide synthesis process according to any one of claims 19 to 21, characterized in that the supports are:
either solubilized in a standard organic solvent such as dichloromethane, tetrahydrofuran, dioxane, acetonitrile, propionitrile, dimethylformamide, dimethylacetamide, N-methyl-pyrrolidone, acetone, toluene, chlorobenzene, dichlorobenzene, nitromethane, nitroethane, or a mixture of these solvents,
or solubilized in an ionic liquid matrix, preferably trimethylbutylammonium triflimidide or [tmba][NTf₂], 1-ethyl-3-methylimidazolium triflimidide or [emim][NTf₂], 1-butyl-3-methylimidazolium triflimidide or [bmim][NTf₂] or any other combination of onium cation and liquid anion at a temperature less than or equal to 100° C., preferably 50° C.,
or solubilized in a mixture comprising an organic solvent and an ionic liquid matrix as defined above.

23. Compounds of formula (I-a)
A⁺-L-R—OW, X⁻
in which:
W represents:
either a hydrogen atom,
or a —COOR₁ group, R₁ representing an alkyl group comprising 1 to 20 carbon atoms or an aryl group comprising 6 to 30 carbon atoms, or a perfluoroalkyl group comprising 1 to 20 carbon atoms, said alkyl or aryl groups being optionally functionalized, R₁ representing in particular —CHCl—CCl₃ or 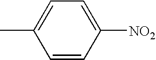
or a group of the following formula (A′): 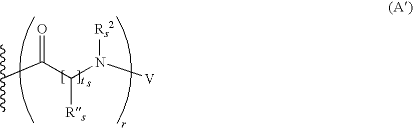
in which:
s is an integer varying from 1 to r,
r is an integer varying from 1 to 20,
tₛ is an integer varying from 1 to 20,
R″ₛ represents an amino acid residue,
Rₛ² represents H or a linear or branched alkyl group, comprising 1 to 20 carbon atoms and being able to form a ring with the R″, group, the nitrogen atom carrying the group Rₛ² and the carbon atom carrying the R″, group, said ring comprising 3 to 20 members, in particular 5 or 6 members,
V represents a hydrogen atom or a protective group of the amine function, with the exception of Boc, in particular Fmoc, Cbz, Z, SO₂R_{g}, R_{g} representing a linear or branched alkyl group comprising 1 to 20 carbon atoms, a substituted or non-substituted aryl group, a perfluoroalkyl group comprising 1 to 20 carbon atoms,
or a group of the following formula (B′): 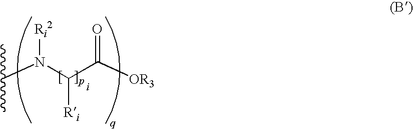
in which:
i is an integer varying from 1 to q,
q is an integer varying from 1 to 20,
pᵢ is an integer varying from 1 to 20,
R′ᵢ represents an amino acid residue,
Rᵢ² represents H or a linear or branched alkyl group, comprising 1 to 20 carbon atoms and being able to form a ring with the R′ᵢ group, the nitrogen atom carrying the group Rᵢ² and the carbon atom carrying the R′ᵢ group, said ring comprising 3 to 20 members, in particular 5 or 6 members,
R₃ representing a hydrogen atom or a protective group of the terminal acid function of the amino acid, and being chosen from one of the following groups: a linear or branched alkyl group, comprising 1 to 20 carbon atoms, in particular methyl or tertiobutyl, a benzyl group or an Si(ORₕ)₃ group, Rₕ representing a linear or branched alkyl group of 1 to 20 carbon atoms, and representing in particular a tertiobutyl group,
or a group of the following formula (C′): 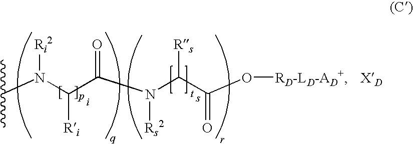
in which:
s, r, tₛ, R″, and Rₛ² are as defined above in formula (A′), and
i, q, pᵢ, R′; and Rᵢ² are as defined above in formula (B′),
X_{D}⁻ represents a functional or non-functional anion, chosen in particular from Cl⁻, Br⁻, I⁻, BF₄⁻, CF₃SO₃⁻, N(SO₂CF₃)₂⁻, PF6⁻, CH₃CO₂⁻, CF₃CO₂⁻, R_{α}CO₂⁻, R_{F}CO₂⁻, R_{α}SO₃⁻, R_{F}SO₃⁻, R_{α}SO₄⁻, (R_{α})₃₋ₓPO₄^{x-}, x representing an integer equal to 1, 2 or 3, AlCl₄⁻, SnCl₃⁻, ZnCl₃⁻, R_{α} representing an alkyl group comprising 1 to 20 carbon atoms, R_{F} representing a perfluoroalkyl group comprising 1 to 20 carbon atoms,
A_{D}⁺ represents a cationic entity, in particular chosen from the pyridinium, imidazolium, ammonium, phosphonium or sulphonium cations, cyclic or non-cyclic, substituted or non-substituted, and preferably ammonium or phosphonium,
L represents an arm, in particular a linear or branched alkyl group, or aralkyl or alkaryl comprising 3 to 20 carbon atoms,
R represents a group chosen from the following groups:
a group of formula —C(Rₐ)(R_{b})—, Rₐ and Rₕ representing independently of one another a hydrogen atom or a linear or branched alkyl group, comprising 1 to 20 carbon atoms, the group of formula —C(Rₐ)(R_{b})— preferably representing a —CH₂—, —CH(Me)— or —C(Me)₂- group,
a group of formula -T-Ar₁—CH(R^{c})—, in which:
T is chosen from one of the following groups: CH₂, O, S and NR_{d}, R_{d} representing a hydrogen atom or a linear or branched alkyl group, comprising 1 to 20 carbon atoms,
Ar₁ represents an aromatic group of the following formula: 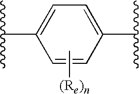
n representing an integer equal to 0, 1, 2, 3 or 4,
Rₑ representing either a linear or branched alkyl group, comprising 1 to 12 carbon atoms, in particular a methyl group, or an alkoxy group comprising 1 to 12 carbon atoms, in particular a methoxy, ethoxy, propyloxy, isopropyloxy, butyloxy, isobutyloxy or tertiobutyloxy group,
R_{c}, represents either a hydrogen atom, or a linear or branched alkyl group, comprising 1 to 20 carbon atoms, or an aromatic group Arₑ of the following formula: 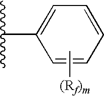
m representing an integer equal to 1, 2, 3, 4 or 5,
R_{f} representing either a linear or branched alkyl group, comprising 1 to 12 carbon atoms, in particular a methyl group, or an alkoxy group comprising 1 to 12 carbon atoms, in particular a methoxy, ethoxy, propyloxy, isopropyloxy, butyloxy, isobutyloxy or tertiobutyloxy group,
A⁺, L, R and X⁻ corresponding to the same definition as that given above for A_{D}⁺, L_{D}, R_{D} and X₆,
A_{D}⁺-L_{D}-R_{D} and A⁺-L-R being able to be identical or different,
and X_{D}⁻ and X⁻ being able to be identical or different,
the following compounds being excluded:

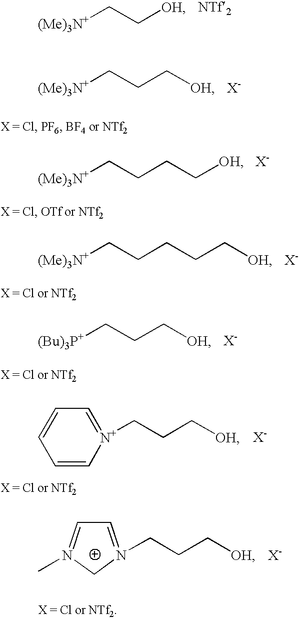

24. Compounds according to claim 23, corresponding to the following formula (I):
A⁺-L-R—OY, X⁻ (I)
in which:
A⁺, X⁻, L and R are as defined in claim 23,
Y represents:
either a hydrogen atom, the salt of formula (I) then comprising a cation functionalized by an alcohol function and corresponding to the following formula (I_{D}): A⁺-L-R—OH, X⁻,
or a —COOR_{I} group, R₁ being as defined in claim 23,
the salt of formula (I) then comprising a cation functionalized by a mixed carbonate function and corresponding to the following formula (I_{I}):

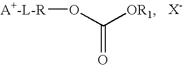

25. Compounds according to claim 23 or 24, corresponding to one of the following formulae: [chemical structure]


SEQUENCE LISTING (1 sequence)

SEQ ID NO: 1 - protein, 5 aa, Artificial Sequence
  Synthetic Peptide
LVVLA